(19) United States
(12) Patent Application Publication
(10) Pub. No.: US 2024/0261259 A1
(43) Pub. Date: Aug. 8, 2024

(54) METHODS AND KITS FOR PREDICTING THE EFFICACY OF MIDOSTAURIN FOR THE TREATMENT OF CANCER

(71) Applicant: KINOMICA LIMITED, Macclesfield, Cheshire (GB)

(72) Inventors: Pedro Rodriguez CUTILLAS, Macclesfield (GB); David James BRITTON, Macclesfield (GB); Weronika Ewa BOREK, Macclesfield (GB); Arran David DOKAL, Macclesfield (GB)

(73) Assignee: KINOMICA LIMITED, Macclesfield, Cheshire (GB)

(21) Appl. No.: 18/561,895

(22) PCT Filed: May 18, 2022
(86) PCT No.: PCT/GB2022/051251

§ 371 Date: Nov. 17, 2023

(30) Foreign Application Priority Data
May 18, 2021 (GB) 2107091.7

(51) Int. Cl. A61K 31/395; A61K 45/06; G01N 33/68; G16H 20/10

(52) U.S. Cl. CPC A61K 31/395; A61K 45/06; G01N 33/6848; G16H 20/10; G01N 2800/52

(57) ABSTRACT

Methods are provided for predicting the efficacy of midostaurin for the treatment of a cancer in an individual subject or a cohort of patients. The method comprises determining a phosphoproteomic and/or a proteomic signature within a sample obtained from the individual subject wherein the phosphoproteomic and/or proteomic signature provides a personalised prediction for the individual subject of the efficacy of midostaurin for treatment of cancer. This invention has utility in methods for treatment of a range of cancers including acute myeloid leukaemia (AML). Companion diagnostic kits and their use in dosage regimens for the treatment of cancer are also provided.

The invention relates generally to a set of proteins and phosphorylation sites that may be used to predict responses of cancer patients to a newly approved anti-cancer drug.

Acute myeloid leukaemia (AML) is a disease with no cure for most patients. Until recently, patients were treated with induction chemotherapy followed by consolidation treatment with cytarabine. More recently, the tyrosine kinase inhibitor midostaurin was approved to treat AML patients positive for mutations in the receptor tyrosine kinase FLT3, which is mutated in about 30% of all AML patients.

Phase 3 clinical trials showed that about 60% of FLT3 mutant-positive patients responded to midostaurin. However, earlier phase 2 trials also showed that about 40% of FLT3 mutant-negative cases also benefited from midostaurin therapies. These results suggest that FLT3 mutation status is not the only determinant in conferring sensitivity to midostaurin. Thus, because of the low specificity and sensitivity of FLT3 mutations as a biomarker of responses to midostaurin, many patients who are treated do not respond to therapy and several individual who could potentially respond are not currently treated with this drug.

Patients are eligible to be treated with midostaurin if they are positive for FLT3 mutations. This is currently determined by using a companion diagnostic (CDx) test based on DNA sequencing of the FLT3 gene to detect internal tandem duplications (ITDs) or point mutations on this gene. However, the current CDx test used to select patients to be treated with midostaurin has low specificity and sensitivity. In addition, there is no guidance on whether any FLT3 positive patient cohorts exist that are non-responsive to midostaurin.

Previous work in the field of AML proteomics includes the phosphoproteins and biomarkers disclosed in WO 2018/234404. The proteomic biomarkers of WO 2018/234404 were identified in cells treated ex-vivo with midostaurin. Casado et al Leukemia 32, 1818-1822 (2018) describes a proteomic and genomic integration study that identified kinase and differentiation determinants of kinase inhibitor sensitivity in leukemia cells. A proportion of such signatures consisted of phosphorylation sites on proteins such as PKC delta and GSK3A. These experiments were carried out ex-vivo; in other words, AML cells were growth in culture conditions in cell culture flasks and their proteomes and phosphoproteomes compared with the sensitivity of cells treated with relatively high doses (10 μM) of midostaurin as monotherapy. These concentrations are larger than the concentration that midostaurin reaches in blood during therapy—i.e. they are outside of the normal pharmacokinetic levels. At present there is no information on phosphorylation sites and proteins associated to responses to midostaurin plus chemotherapy in clinically relevant patient cohorts.

WO 2016/057705 discloses methods of using biomarkers for use in connection with diagnosis and treatment of cancer with CAR-expressing cell therapy.

Gerdes et al Nature Communications 12, 1850 (2021) describes how drug ranking using machine learning systematically predicts the efficacy of anti-cancer drugs.

### SUMMARY OF THE INVENTION

In a first aspect, the invention provides a method for predicting the efficacy of midostaurin for the treatment of a cancer in an individual subject, the method comprising determining a phosphoproteomic signature within a sample obtained from the individual subject wherein the phosphoproteomic signature provides a personalised prediction for the individual subject of the efficacy of midostaurin for treatment of cancer.

Suitably, in embodiments of the invention the cancer may be selected from the group consisting of: acute myeloid leukemia (AML); high-risk myeloid dysplastic syndrome (MDS); aggressive systemic mastocytosis (ASM); systemic mastocytosis with associated hematological neoplasm (SM-AHN); and mast cell leukemia (MCL).

In a second aspect, the invention provides method for predicting the efficacy of midostaurin for the treatment of a cancer in an individual subject, the method comprising determining a proteomic signature within a sample obtained from the individual subject wherein the proteomic signature provides a personalised prediction for the individual subject of the efficacy of midostaurin for treatment of cancer.

In a third aspect, the invention provides midostaurin for use in the treatment of acute myeloid leukemia (AML); high-risk myeloid dysplastic syndrome (MDS); aggressive systemic mastocytosis (ASM); systemic mastocytosis with associated hematological neoplasm (SM-AHN); or mast cell leukemia (MCL) in a FLT3 mutant-negative individual subject, wherein the individual subject is identified as suitable for treatment with midostaurin by determining a phosphoproteomic signature within a sample obtained from the individual subject wherein the phosphoproteomic signature provides a personalised indication that the individual subject is a suitable candidate for treatment.

In a fourth aspect, the invention provides midostaurin for use in the treatment of acute myeloid leukemia (AML); high-risk myeloid dysplastic syndrome (MDS): aggressive systemic mastocytosis (ASM); systemic mastocytosis with associated hematological neoplasm (SM-AHN); or mast cell leukemia (MCL) in a FLT3 mutant-negative individual subject, wherein the individual subject is identified as suitable for treatment with midostaurin by determining a proteomic signature within a sample obtained from the individual subject wherein the proteomic signature provides a personalised indication that the individual subject is a suitable candidate for treatment.

In a fifth aspect, the invention provides dosage regimen for cancer therapy, comprising administering to an individual subject midostaurin orally as either a 50 mg dose twice daily or a 100 mg dose twice daily,
wherein the individual subject is identified as suitable for treatment with midostaurin by determining a proteomic and/or a phosphoproteomic signature within a sample obtained from the individual subject wherein the proteomic and/or phosphoproteomic signature provides a personalised indication that the individual subject is a suitable candidate for treatment.

In a sixth aspect, the invention provides a method of treating cancer in a FLT3 mutant-negative or mutant-positive individual subject, wherein the individual subject is identified as suitable for treatment with midostaurin by obtaining a sample from the individual subject and determining a phosphoproteomic signature within the sample obtained from the individual subject wherein the phosphoproteomic signature provides a personalised indication that the individual subject is a suitable candidate for treatment.

In a seventh aspect, the invention provides a method of treating cancer in a FLT3 mutant-negative or mutant-positive individual subject, wherein the individual subject is identified as suitable for treatment with midostaurin by obtaining a sample from the individual subject and determining a proteomic signature within the sample obtained from the individual subject wherein the proteomic signature provides a personalised indication that the individual subject is a suitable candidate for treatment.

In an eighth aspect, the invention provides a companion diagnostic assay kit, wherein the kit comprises one or more affinity reagents selected from: an aptamer; a molecularly imprinted polymer; and an antibody or an antigen binding fragment or mimetic thereof, and wherein the kit is configured to determine a phosphoproteomic signature within a sample obtained from an individual subject, and wherein the phosphoproteomic signature provides a personalised indication that the individual subject is a suitable candidate for treatment of a cancer with midostaurin.

In a ninth aspect, the invention provides a companion diagnostic assay kit, wherein the kit comprises one or more affinity reagents selected from: an aptamer; a molecularly imprinted polymer; and an antibody or an antigen binding fragment or mimetic thereof, and wherein the kit is configured to determine a proteomic signature within a sample obtained from an individual subject, and wherein the proteomic signature provides a personalised indication that the individual subject is a suitable candidate for treatment of a cancer with midostaurin.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### REFERENCE IS MADE TO A NUMBER OF FIGURES AS FOLLOWS

FIG. 1. Overview of study and sample set.

FIG. 2. Responses to midostaurin plus chemotherapy as a function of age, allogeneic transplantation, cell differentiation markers and genetic mutations.

FIG. 3. Selected phosphopeptides correlated with responses to midostaurin plus chemotherapy.

FIG. 4. Phosphorylation of selected phosphopeptide markers by response to midostaurin plus chemotherapy.

FIG. 5. Examples of differential survival analysis based on the named phosphosite markers or models.

FIG. 6. Examples of differential survival analysis based on the named protein markers or models.

FIG. 7. A plot showing results of a principal component analysis—positive and negative samples.

FIG. 8. A plot showing results of a principal component analysis—multiple sub-groups of positive samples.

FIG. 9. A plot showing results of a principal component analysis—multiple sub-groups of positive samples with negative samples removed.

### DETAILED DESCRIPTION OF THE INVENTION

The present invention relates generally to a set of proteins, that contribute to proteomic signatures, and/or phosphorylation sites, that contribute to phosphorylomic signatures, that may be used to predict responses of cancer patients to treatment with midostaurin.

The markers of the present invention were identified in samples taken from patients that subsequently undertook treatment with midostaurin plus chemotherapy. The present invention therefore provides proteomic and/or phosphoproteomic signatures that are associated with clinical responses to midostaurin. These signatures predict responses to this drug with greater precision than the clinically approved genetic marker that is currently used to make clinical decisions. These signatures, therefore, may find application in clinical assays to direct patients for therapies based on midostaurin irrespective of their FLT3 mutational status. Biomarkers in these signatures may be predictive of responses by themselves but the predictive accuracy increases when these are combined in a pairwise manner or in machine learning models trained from these data.

As used herein, references to midostaurin include midostaurin treatment as monotherapy as well as part of a combination therapy, such as in combination with chemotherapy. As used herein, references to “midostaurin” may therefore be substituted for “a combination therapy comprising midostaurin”, “midostaurin and chemotherapy” or “a combination treatment comprising midostaurin and chemotherapy”. The method may be a method of predicting the efficacy of midostaurin and chemotherapy for treatment of a cancer in a patient. The chemotherapy may be cytarabine, doxorubicin, idarubicin and/or daunorubicin. The chemotherapy may be daunorubicin & cytarabine. The chemotherapy may be cytarabine.

As used herein the term “combination therapy” includes any combination of midostaurin with one or more further active ingredients.

The one or more proteins and/or the level of phosphorylation at the one or more phosphorylation sites may be termed “biomarkers” or components of a “signature” or a plurality of “signatures” herein.

As used herein, the term “one or more” embraces any integer from one up to and including the full number of biomarkers referenced. For example “one or more” may refer to any one, or two, or three, or four, or five, or six, or seven, or eight, or nine, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 38, or 37, or 38, or 39, or 40, or 41, or 42, or 43, or 44, or 45, or 46, or all of the biomarkers referenced.

When it is predicted the cancer in the patient may be effectively treated with midostaurin, the patient may be said to have a midostaurin-responsive phenotype. As used herein the term “midostaurin-responsive phenotype” and “midostaurin-responder phenotype” are used interchangeably. The midostaurin-responsive phenotype may alternatively be termed a midostaurin-responsive signature. The midostaurin-responsive phenotype may be a proteomic and/or phosphoproteomic phenotype. The patient may therefore be said to have a midostaurin-responsive proteomic phenotype and/or a midostaurin-responsive phosphoproteomic phenotype. The terms “phenotype” and “signature” may be used interchangeably. The patient may be said to have a midostaurin-responsive proteomic signature and/or a midostaurin-responsive phosphoproteomic signature. The proteomic phenotype may be defined by the level of the one or more proteins in the sample. The phosphoproteomic phenotype may be defined by the level of phosphorylation at the one or more phosphorylation sites in the sample. The midostaurin-responsive proteomic phenotype and/or midostaurin-responsive phosphoproteomic phenotype may therefore be determined in a sample according to step (a) of the method. The midostaurin-responsive proteomic phenotype may be determined by performing a proteomic assay on the sample from the patient. The proteomic assay may comprise determining the level of one or more of the proteins referred to herein via the method. The midostaurin-responsive phosphoproteomic phenotype may be determined by performing a phosphoproteomic assay on the sample from the patient. The phosphoproteomic assay may comprise determining the level of phosphorylation at one or more phosphorylation sites referred to herein via the method.

As used herein, references to the level of the one or more proteins may refer to the expression level of the one or more proteins and vice versa: the terms are used interchangeably. References to expression of one or more proteins, at a “high level” (or a level that is high), as used here and elsewhere in the specification, denote a level of expression which is higher than the average level of expression of the relevant proteins. References to a “low level” of expression (or a level that is low) similarly denote a level of expression which is the same as or less than the average level of expression of the proteins. The average level of expression of the proteins is a standardised value which may be determined by reference to an average calculated across a plurality of samples, or by reference to the level of expression of the proteins in undifferentiated myeloblasts or other healthy cell types, which may be established either by laboratory analysis according to methods well known in the art (including LC-MS/MS), or by reference to information available in the art. Thus, for example, the average level of expression of the proteins may be determined by establishing the range of expression levels of the proteins in cell samples obtained from a large number of cancer patients, and calculating the mean level of expression across the samples. A “high level” of expression is a level of expression which is higher than the calculated median or mean or upper quartile or threshold level. A “low level” of expression is a level of expression which is lower than the calculated median or mean or lower quartile or threshold level. A level of expression which is “not high” is a level of expression which is not higher than the calculated median or mean or upper quartile or threshold level; for example, the level of expression may be about or lower than the calculated median or mean or lower quartile or threshold level.

References to phosphorylation at a “high level” (or a level that is high), as used here and elsewhere in the specification, denote a level of phosphorylation which is higher than the average phosphorylation of the relevant protein or at the relevant phosphorylation site. References to a “low level” of phosphorylation similarly denote a level of phosphorylation which is the same as or less than the average phosphorylation of the relevant protein or at the relevant phosphorylation site. The average phosphorylation of the relevant protein or the relevant phosphorylation site is a standardised value which may be determined by reference to an average calculated across a plurality of samples, or by reference to the phosphorylation state of the relevant protein or the relevant phosphorylation site in undifferentiated myeloblasts or other healthy cell types, which may be established either by laboratory analysis according to methods well known in the art (including LC-MS/MS), or by reference to information available in the art. Thus, for example, the average level of phosphorylation at a particular phosphorylation site may be determined by establishing the range of phosphorylation at that site in cell samples obtained from a large number of cancer patients, and calculating the mean phosphorylation across the samples. A “high level” of phosphorylation at that site is a level of phosphorylation which is higher than the calculated median or mean or upper quartile or threshold level. A “low level” of phosphorylation at that site is a level of phosphorylation which is lower than the calculated median or mean or lower quartile or threshold level. A level of phosphorylation which is “not high” is a level of phosphorylation which is not higher than the calculated median or mean or upper quartile or threshold level; for example, the level of phosphorylation may be about or lower than the calculated median or mean or lower quartile or threshold level.

The biomarkers may predict that a cancer, such as acute myeloid leukaemia (AML), in the patient may be effectively treated with midostaurin when the level of the one or more biomarker is high. In other words, the one or more biomarker may be increased in patients with a midostaurin-responsive phenotype.

In one embodiment the method may comprise predicting that a cancer, such as an acute myeloid leukaemia, in the patient may be effectively treated with midostaurin when:
it is determined that there is a high level of one or more proteins selected from the group consisting of
   Integrin alpha-5;
   Glutathione S-transferase theta-2;
   Arginine-tRNA ligase, cytoplasmic;
   Filensin;
   Protein unc-13 homolog D;
   V-type proton ATPase subunit B, brain isoform;
   Alpha-1,6-mannosyl-glycoprotein 2-beta-N-acetylglucosaminyltransferase;
   Arfaptin-1;
   C—C motif chemokine 3;
   UDP-N-acetylhexosamine pyrophosphorylase-like protein 1;
   Eukaryotic translation initiation factor 2 subunit 3;
   Glutathione synthetase;
   V-type proton ATPase catalytic subunit A;
   Sodium/potassium-transporting ATPase subunit alpha-1;
   Serine/threonine-protein kinase MRCK beta;
   Protein YIPF4;
   DnaJ homolog subfamily C member 2;
   Immunoglobulin lambda-1 light chain;
   (Lyso)-N-acylphosphatidylethanolamine lipase;
   Ribosome biogenesis protein BMS1 homolog;
   Rho GTPase-activating protein 26; and
   Patatin-like phospholipase domain-containing protein 6;
      and/or
it is determined that there is a low level or not a high level of one or more proteins selected from the group consisting of:
   Probable rRNA-processing protein EBP2;
   RNA exonuclease 4;
   SAP30-binding protein;
   2-aminomuconic semialdehyde dehydrogenase;
   Extracellular matrix protein 1;
   CCA tRNA nucleotidyltransferase 1, mitochondrial;
   BTB/POZ domain-containing protein 16;
   Kalirin;
   Ribosomal oxygenase 1;
   Oxysterol-binding protein-related protein 9;
   ATP-dependent DNA helicase Q1;
   Syntaxin-12;
   Protein MEMO1;
   Caspase recruitment domain-containing protein 19;
   Calcium/calmodulin-dependent protein kinase type 1D;
   Adhesion G protein-coupled receptor A1; and
   AMP deaminase 3;
      and/or
it is determined that there is a high level of phosphorylation at one or more phosphorylation sites selected from the group consisting of:
   T719 of Signal transducer and activator of transcription 1-alpha/beta;
   T729 and/or S730 of Glycogen[starch] synthase, muscle;
   T77 of Eukaryotic translation initiation factor 4E-binding protein 1;
   T46 of Eukaryotic translation initiation factor 4E-binding protein 2;
   S302 of Protein kinase C delta type;
   S183 of Proline-rich AKT1 substrate 1;
   T19 of Glycogen synthase kinase-3 alpha;
   S363 of Serine/threonine-protein kinase B-raf;
   S2996 of Serine-protein kinase ATM;
   T327 of Vimentin;
   S447 of Epsin-1;
   S310 of Caspase-9; and
   T120 of Myristoylated alanine-rich C-kinase substrate;
      and/or
it is determined that there is a low level or not a high level of phosphorylation at one or more phosphorylation sites selected from the group consisting of:
   S627 of Zinc finger protein 608;
   T58 of Myc proto-oncogene protein and/or T58 of N-myc proto-oncogene protein;
   S316 of Core histone macro-H2A. 1;
   S1029 of Lysine-specific demethylase 2B;
   S3620 of Histone-lysine N-methyltransferase 20;
   S15 and/or S17 of Tyrosine-protein kinase JAK3;
   S1009 of PH and SEC7 domain-containing protein 3;
   S1054 of Cyclin-dependent kinase 13;
   S244 of DEP domain-containing mTOR-interacting protein;
   S772 of Misshapen-like kinase 1; and
   S179 of Calcium/calmodulin-dependent protein kinase type 1D; and
   S506 of Protein kinase C delta type.

Any biomarker disclosed herein of which a low level may be used to predict that that the cancer in the patient may be effectively treated with midostaurin may alternatively be used to predict that that the cancer in the patient may not be effectively treated with midostaurin when the biomarker is at a high level. In other words, such biomarkers may be increased in patients with a midostauin-non-responsive phenotype.

Any biomarker disclosed herein of which a low level may be used to predict that that the cancer in the patient may be effectively treated with midostaurin (in particular biomarkers shown herein to be increased in non-responders) may alternatively be used to predict that that the cancer in the patient may be effectively treated with midostaurin when the level of said biomarker is not high.

Any biomarker disclosed herein of which a high level may be used to predict that that the cancer in the patient may be effectively treated with midostaurin may alternatively be used to predict that that the cancer in the patient may not be effectively treated with midostaurin when the biomarker is at a low level. In other words, such biomarkers may be decreased in patients with a midostauin-non-responsive phenotype.

In a specific embodiment of the invention the method may comprise determining a protein signature for a patient in a sample obtained from that patient. The protein signature may be comprised of protein biomarkers comprising the following group:
Heterogeneous nuclear ribonucleoprotein M
Protein PML (E3 SUMO-protein ligase PML) (EC 2.3.2.-) (Promyelocytic leukemia protein) (RING finger protein 71) (RING-type E3 SUMO transferase PML) (Tripartite motif-containing protein 19) (TRIM19)
Neuroblast differentiation-associated protein AHNAK
Myoferlin
Dedicator of cytokinesis protein 10 (Zizimin-3)
Eukaryotic translation initiation factor 3 subunit D
Glutamine-fructose-6-phosphate aminotransferase
Choline-phosphate cytidylyltransferase A
V-type proton ATPase subunit B, brain isoform
Eukaryotic translation initiation factor 2 subunit 3
V-type proton ATPase catalytic subunit A

In a further embodiment, the method may comprise determining a protein signature for a patient in a sample obtained from that patient. The protein signature may be comprised of protein biomarkers comprising the following group: Probable rRNA-processing protein EBP2;
Integrin alpha-5;
RNA exonudease 4;
Glutathione S-transferase theta-2;
SAP30-binding protein;
Arginine-tRNA ligase, cytoplasmic;
2-aminomuconic semialdehyde dehydrogenase;
Filensin;
Protein unc-13 homolog D;
V-type proton ATPase subunit B, brain isoform;
Alpha-1,6-mannosyl-glycoprotein 2-beta-N-acetylglucosaminyltransferase;
Arfaptin-1;
C—C motif chemokine 3; and
Extracellular matrix protein 1.

In another embodiment the method may comprise determining in a sample from the patient:
(i) the level of one or more proteins selected from the group consisting of:
   Probable rRNA-processing protein EBP2;
   Integrin alpha-5;
   RNA exonudease 4;
   Glutathione S-transferase theta-2;
   SAP30-binding protein;
   Arginine-tRNA ligase, cytoplasmic;
   2-aminomuconic semialdehyde dehydrogenase; and
   Filensin.

The method may comprise determining in a sample from the patient:
(i) the level of Probable rRNA-processing protein EBP2.

The predictive value of the proteomic signatures utilised in the methods of the invention may be expanded to include a wider range of protein biomarkers. Inclusion of one or more of these proteomic biomarkers may increase the sensitivity or predictive ability of the methods.
Heterogeneous nuclear ribonucleoprotein M
Protein PML (E3 SUMO-protein ligase PML) (EC 2.3.2.-) (Promyelocytic leukemia
protein) (RING finger protein 71) (RING-type E3 SUMO transferase PML)
(Tripartite motif-containing protein 19) (TRIM19)
Neuroblast differentiation-associated protein AHNAK
Myoferlin
Dedicator of cytokinesis protein 10 (Zizimin-3)
Eukaryotic translation initiation factor 3 subunit D
Glutamine-fructose-8-phosphate aminotransferase
Choline-phosphate cytidylyttransferase A
V-type proton ATPase subunit B, brain isoform
Eukaryotic translation initiation factor 2 subunit 3
V-type proton ATPase catalytic subunit A
Probable rRNA-processing protein EBP2
Integrin alpha-5
RNA exonuclease 4
10 Glutathione S-transferase theta-2
SAP30-binding protein
Arginine-tRNA ligase, cytoplasmic
2-aminomuconic semialdehyde dehydrogenase
Filensin
Protein unc-13 homolog D
Alpha-1,6-mannosyl-glycoprotein 2-beta-N-acetylglucosaminyltransferase
Arfaptin-1
C—C motif chemokine 3
Extracellular matrix protein 1
UDP-N-acetylhexosamine pyrophosphorylase-like protein 1
pre-rRNA 2′-O-ribose RNA methyttransferase FTSJ3
Transcription factor jun-B
Fructose-1,6-bisphosphatase 1
Glutathione synthetase
Adenosine deaminase
CCA tRNA nucleotidyitransferase 1, mitochondrial
Sodium/potassium-transporting ATPase subunit alpha-1
Toll-like receptor 2
BTB/POZ domain-containing protein 16
Transcriptional repressor protein YY1
High mobility group protein B3
Ras GTPase-activating protein nGAP
Kalirin
Serine/threonine-protein kinase MRCK beta
Ribosomal oxygenase 1
Oxysterol-binding protein-related protein 9
40 Protein YIPF4
ATP-dependent DNA helicase Q1
DnaJ homolog subfamily C member 2
Syntaxin-12
Protein MEMO1
Caspase recruitment domain-containing 5 protein 19
Immunoglobulin lambda-1 light chain
Calcium/calmodulin-dependent protein kinase type 1D
(Lyso)-N-acylphosphatidylethanolamine lipase
Ribosome biogenesis protein BMS1 homolog
10 Rho GTPase-activating protein 26
Patatin-like phospholipase domain-containing protein 6
Adhesion G protein-coupled receptor A1
AMP deaminase 3

In embodiments of the invention the method may comprise determining a protein phosphorylation signature for a patient in a sample obtained from that patient. The protein phosphorylation signature may be comprised of biomarkers comprising phosphorylated sites comprised within following Table 1:

TABLE 1

| Phosphorylation site(s) - | | |
|---|---|---|
| Protein(site) | Uniprot_ID | Protein name |
| TP53BP1(T382); | TP53B_HUMAN | TP53-binding protein 1 (53BP1) |
| | | (p53-binding protein 1) (p53BP1) |
| MLF2(S240); | MLF2_HUMAN | Myeloid leukemia factor 2 |
| | | (Myelodysplasia-myeloid leukemia |
| | | factor 2) |
| MEFV(S248); | MEFV_HUMAN | Pyrin (Marenostrin) |
| AHNAK(S3426); | AHNK_HUMAN | Neuroblast differentiation-associated |
| | | protein AHNAK (Desmoyokin) |
| NHSL2(S210); | NHSL2_HUMAN | NHS-like protein 2 |
| SYK(S295); | KSYK_HUMAN | Tyrosine-protein kinase SYK |
| SYK(S297); | | (EC 2.7.10.2) (Spleen tyrosine |
| | | kinase) (p72-Syk) |
| LARP1(S90); | LARP1_HUMAN | La-related protein 1 (La |
| | | ribonucleoprotein domain |
| | | family member 1) |
| SVIL(S228); | SVIL_HUMAN | Supervillin (Archvillin) (p205/ |
| | | p250) |
| CCDC86(S24); | CCD86_HUMAN | Coiled-coil domain-containing |
| | | protein 86 (Cytokine-induced |
| | | protein with coiled-coil domain) |
| PFKFB2(S486); | F262_HUMAN | 6-phosphofructo-2-kinase/fructose- |
| | | 2,6-bisphosphatase 2 (6PF-2-K/Fru- |
| | | 2,6-P2ase 2) (PFK/FBPase 2) (6PF- |
| | | 2-K/Fru-2,6-P2ase heart-type isozyme) |
| | | [Includes: 6-phosphofructo-2-kinase |
| | | (EC 2.7.1.105); Fructose-2,6- |
| | | bisphosphatase (EC 3.1.3.46)] |
| SHANK3(S1650); | SHAN3_HUMAN | SH3 and multiple ankyrin repeat |
| SHANK3(S1654); | | domains protein 3 (Shank3) (Proline- |
| | | rich synapse-associated protein |
| | | 2) (ProSAP2) |
| SRRM2(S1878); | SRRM2_HUMAN | Serine/arginine repetitive matrix |
| SRRM2(T1880); | | protein 2 (300 kDa nuclear matrix |
| | | antigen) (Serine/arginine-rich |
| | | splicing factor-related nuclear |
| | | matrix protein of 300 kDa) (SR- |
| | | related nuclear matrix protein of |
| | | 300 kDa) (Ser/Arg-related nuclear |
| | | matrix protein of 300 kDa) (Splicing |
| | | coactivator subunit SRm300) (Tax- |
| | | responsive enhancer element-binding |
| | | protein 803) (TaxREB803) |
| KIAA0528(S297); | C2CD5_HUMAN | C2 domain-containing protein 5 (C2 |
| | | domain-containing phosphoprotein |
| | | of 138 kDa) |
| KIAA1429(S138); | VIR_HUMAN | Protein virilizer homolog |
| MAP1A(S1157); | MAP1A_HUMAN | Microtubule-associated protein 1A |
| | | (MAP-1A) (Proliferation-related |
| | | protein p80) [Cleaved into: MAP1A |
| | | heavy chain; MAP1 light chain LC2] |
| CAST(T245); | ICAL_HUMAN | Calpastatin (Calpain inhibitor) |
| | | (Sperm BS-17 component) |
| CBFA2T3(S459); | MTG16_HUMAN | Protein CBFA2T3 (MTG8-related protein |
| | | 2) (Myeloid translocation gene on |
| | | chromosome 16 protein) (hMTG16) |
| | | (Zinc finger MYND domain-containing |
| | | protein 4) |
| NDRG1(S378); | NDRG1_HUMAN | Protein NDRG1 (Differentiation- |
| | | related gene 1 protein) (DRG-1) |
| | | (N-myc downstream-regulated gene |
| | | 1 protein) (Nickel-specific |
| | | induction protein Cap43) (Reducing |
| | | agents and tunicamycin-responsive |
| | | protein) (RTP) (Rit42) |
| ULK1(S477); | ULK1_HUMAN | Serine/threonine-protein kinase |
| ULK1(S479); | | ULK1 (EC 2.7.11.1) (Autophagy-related |
| | | protein 1 homolog) (ATG1) (hATG1) |
| | | (Unc-51-like kinase 1) |
| DEPTOR(S244); | DPTOR_HUMAN | DEP domain-containing mTOR-interacting |
| | | protein (DEP domain-containing protein |
| | | 6) |
| BPTF(S1251); | BPTF_HUMAN | Nucleosome-remodeling factor subunit |
| | | BPTF (Bromodomain and PHD finger- |
| | | containing transcription factor) |
| | | (Fetal Alz-50 clone 1 protein) |
| | | (Fetal Alzheimer antigen) |
| MAPKBP1(S1263); | MABP1_HUMAN | Mitogen-activated protein kinase- |
| | | binding protein 1 (JNK-binding |
| | | protein 1) (JNKBP-1) |
| REEP4(S152); | REEP4_HUMAN | Receptor expression-enhancing protein |
| | | 4 |
| ZNF408(T322); | ZN408_HUMAN | Zinc finger protein 408 (PR domain |
| | | zinc finger protein 17) |
| EFR3A(T224); | EFR3A_HUMAN | Protein EFR3 homolog A (Protein |
| | | EFR3-like) |
| SRRM2(S2044); | SRRM2_HUMAN | Serine/arginine repetitive matrix |
| SRRM2(S2046); | | protein 2 (300 kDa nuclear matrix |
| | | antigen) (Serine/arginine-rich |
| | | splicing factor-related nuclear |
| | | matrix protein of 300 kDa) (SR- |
| | | related nuclear matrix protein of |
| | | 300 kDa) (Ser/Arg-related nuclear |
| | | matrix protein of 300 kDa) (Splicing |
| | | coactivator subunit SRm300) (Tax- |
| | | responsive enhancer element-binding |
| | | protein 803) (TaxREB803) |
| IRAK3(S112); | IRAK3_HUMAN | Interleukin-1 receptor-associated |
| | | kinase 3 (IRAK-3) (IL-1 receptor- |
| | | associated kinase M) (IRAK-M) |
| | | (Inactive IL-1 receptor-associated |
| | | kinase 3) |
| PPP1R9B(S100); | NEB2_HUMAN | Neurabin-2 (Neurabin-II) (Protein |
| | | phosphatase 1 regulatory subunit |
| | | 9B) (Spinophilin) |
| SGK223(S694); | PRAG1_HUMAN | Inactive tyrosine-protein kinase |
| | | PRAG1 (PEAK1-related kinase-activating |
| | | pseudokinase 1) (Pragmin) (Sugen |
| | | kinase 223) (SgK223) |
| LILRB3(S503); | LIRB3_HUMAN | Leukocyte immunoglobulin-like |
| | | receptor subfamily B member 3 |
| | | (LIR-3) (Leukocyte immunoglobulin- |
| | | like receptor 3) (CD85 antigen- |
| | | like family member A) (Immunoglobulin- |
| | | like transcript 5) (ILT-5)(Monocyte |
| | | inhibitory receptor HL9) (CD antigen |
| | | CD85a) |
| HIDE1(S217); | HIDE1_HUMAN | Protein HIDE1 |
| HIDE1(T220); | | |
| SRRM1(T220); | SRRM1_HUMAN | Serine/arginine repetitive matrix |
| | | protein 1 (SR-related nuclear matrix |
| | | protein of 160 kDa) (SRm160) (Ser/Arg- |
| | | related nuclear matrix protein) |
| BRAT1(S747); | BRAT1_HUMAN | BRCA1-associated ATM activator 1 |
| | | (BRCA1-associated protein required for |
| | | ATM activation protein 1) |
| TLE3(S293); | TLE3_HUMAN | Transducin-like enhancer protein 3 |
| | | (Enhancer of split groucho-like |
| | | protein 3) (ESG3) |
| EGLN1(S125); | EGLN1_HUMAN | Egl nine homolog 1 (EC 1.14.11.29) |
| | | (Hypoxia-inducible factor proly! |
| | | hydroxylase 2) (HIF-PH2) (HIF-prolyl |
| | | hydroxylase 2) (HPH-2) (Prolyl |
| | | hydroxylase domain-containing protein 2) |
| | | (PHD2) (SM-20) |
| NDRG1(S333); | NDRG1_HUMAN | Protein NDRG1 (Differentiation-related |
| NDRG1(T335); | | gene 1 protein) (DRG-1) (N-myc |
| | | downstream-regulated gene 1 protein) |
| | | (Nickel-specific induction protein |
| | | Cap43) (Reducing agents and tunicamycin- |
| | | responsive protein) (RTP) (Rit42) |
| NUMB(T251); | NUMB_HUMAN | Protein numb homolog (h-Numb) |
| NUMB(S252); | | (Protein S171) |
| SEC61B(S19); | SC61B_HUMAN | Protein transport protein Sec61 subunit |
| | | beta |
| TFPT(S252); | TFPT_HUMAN | TCF3 fusion partner (INO80 complex |
| | | subunit F) (Protein FB1) |
| CCDC86(S50); | CCD86_HUMAN | Coiled-coil domain-containing protein 86 |
| | | (Cytokine-induced protein with coiled- |
| | | coil domain) |
| ZMYM3(S464); | ZMYM3_HUMAN | Zinc finger MYM-type protein 3 (Zinc |
| | | finger protein 261) |
| TLK2(S771); | TLK2_HUMAN | Serine/threonine-protein kinase tousled- |
| | | like 2 (EC 2.7.11.1) (HsHPK) (PKU-alpha) |
| | | (Tousled-like kinase 2) |
| SEPT6(T418); | SEPT6_HUMAN | Septin-6 |
| IL1RAP(S557); | IL1AP_HUMAN | Interleukin-1 receptor accessory protein |
| IL1RAP(S563); | | (IL-1 receptor accessory protein) (IL- |
| | | 1RAcP) (EC 3.2.2.6) (Interleukin-1 |
| | | receptor 3) (IL-1R-3) (IL-1R3) |
| PELP1(T749); | PELP1_HUMAN | Proline-, glutamic acid- and leucine-rich |
| | | protein 1 (Modulator of non-genomic |
| | | activity of estrogen receptor) |
| | | (Transcription factor HMX3) |
| DUS3L(S276); | DUS3L_HUMAN | tRNA-dihydrouridine(47) synthase |
| DUS3L(S277); | | [NAD(P)(+)]-like (EC 1.3.1.-—) (tRNA- |
| | | dihydrouridine synthase 3-like) |
| EVI2B(T273); | EVI2B_HUMAN | Protein EVI2B (Ecotropic viral integration |
| | | site 2B protein homolog) (EVI-2B) (CD |
| | | antigen CD361) |
| DFFA(S315); | DFFA_HUMAN | DNA fragmentation factor subunit alpha |
| | | (DNA fragmentation factor 45 kDa |
| | | subunit) (DFF-45) (Inhibitor of CAD) |
| | | (ICAD) |
| DOCK8(S913); | DOCK8_HUMAN | Dedicator of cytokinesis protein 8 |
| YEATS2(S536); | YETS2_HUMAN | YEATS domain-containing protein 2 |
| GSE1(S909); | GSE1_HUMAN | Genetic suppressor element 1 |
| GAB2(S147); | GAB2_HUMAN | GRB2-associated-binding protein 2 |
| | | (GRB2-associated binder 2) (Growth |
| | | factor receptor bound protein 2- |
| | | associated protein 2) (pp100) |
| CHAMP1(S204); | CHAP1_HUMAN | Chromosome alignment-maintaining |
| | | phosphoprotein 1 (Zinc finger protein |
| | | 828 |
| CRKL(T120); | CRKL_HUMAN | Crk-like protein |
| TOP2B(S1478); | TOP2B_HUMAN | DNA topoisomerase 2-beta (EC 5.6.2.2) |
| | | (DNA topoisomerase Il, beta isozyme) |
| EPS15(S681); | EPS15_HUMAN | Epidermal growth factor receptor |
| | | substrate 15 (Protein Eps15) (Protein AF- |
| | | 1p) |
| PDS5A(S1305); | PDS5A_HUMAN | Sister chromatid cohesion protein PDS5 |
| | | homolog A (Cell proliferation-inducing |
| | | gene 54 protein) (Sister chromatid |
| | | cohesion protein 112) (SCC-112) |
| AHNAK(S5110); | AHNK_HUMAN | Neuroblast differentiation-associated |
| | | protein AHNAK (Desmoyokin) |
| FAM129B(S696); | NIBA2_HUMAN | Protein Niban 2 (Meg-3) (Melanoma |
| | | invasion by ERK) (MINERVA) (Niban-like |
| | | protein 1) (Protein FAM129B) |
| TTC33(S197); | TTC33_HUMAN | Tetratricopeptide repeat protein 33 (TPR |
| | | repeat protein 33) (Osmosis-responsive |
| | | factor) |
| RANBP2(T1396); | RBP2_HUMAN | E3 SUMO-protein ligase RanBP2 (EC |
| RANBP2(S1400); | | 2.3.2.—) (358 kDa nucleoporin) (Nuclear |
| | | pore complex protein Nup358) |
| | | (Nucleoporin Nup358) (Ran-binding |
| | | protein 2) (RanBP2) (p270) |
| AHNAK(S93); | AHNK_HUMAN | Neuroblast differentiation-associated |
| | | protein AHNAK (Desmoyokin) |
| ENO1(Y44); | ENOG_HUMAN | Gamma-enolase (EC 4.2.1.11) (2- |
| ENO3(Y44); | | phospho-D-glycerate hydro-lyase) |
| ENO2(Y44); | | (Enolase 2) (Neural enolase) (Neuron- |
| | | specific enolase) (NSE) |
| RCC1(S11); | RCC1_HUMAN | Regulator of chromosome condensation |
| | | (Cell cycle regulatory protein) |
| | | (Chromosome condensation protein 1) |
| TLN1(S1021); | TLN1_HUMAN | Talin-1 |
| ZNF768(S83); | ZN768_HUMAN | Zinc finger protein 768 |
| PPHLN1(T204); | PPHLN_HUMAN | Periphilin-1 (CDC7 expression repressor) |
| PPHLN1(S205); | | (CR) (Gastric cancer antigen Ga50) |
| AHNAK(T4100); | AHNK_HUMAN | Neuroblast differentiation-associated |
| | | protein AHNAK (Desmoyokin) |
| PRKAG2(S196); | AAKG2_HUMAN | 5′-AMP-activated protein kinase subunit |
| | | gamma-2 (AMPK gamma2) (AMPK |
| | | subunit gamma-2) (H91620p) |
| CORO7(S21); | CORO7_HUMAN | Coronin-7 (Crn7) (70 kDa WD repeat |
| | | tumor rejection antigen homolog) |
| EIF4EBP1(S85); | 4EBP1_HUMAN | Eukaryotic translation initiation factor 4E- |
| EIF4EBP1(S86); | | binding protein 1 (4E-BP1) (elF4E-binding |
| | | protein 1) (Phosphorylated heat- and |
| | | acid-stable protein regulated by insulin 1) |
| | | (PHAS-I) |
| EIF4EBP2(Y34); | 4EBP2_HUMAN | Eukaryotic translation initiation factor 4E- |
| EIF4EBP2(T46); | | binding protein 2 (4E-BP2) (eIF4E-binding |
| | | protein 2) |
| AKT1S1(S183); | AKTS1_HUMAN | Proline-rich AKT1 substrate 1 (40 kDa |
| | | proline-rich AKT substrate) |
| ATM(S2996); | ATM_HUMAN | Serine-protein kinase ATM (EC 2.7.11.1) |
| | | (Ataxia telangiectasia mutated) (A-T |
| | | mutated) |
| BRAF(S363); | BRAF_HUMAN | Serine/threonine-protein kinase B-raf (EC |
| | | 2.7.11.1) (Proto-oncogene B-Raf) (p94) |
| | | (v-Raf murine sarcoma viral oncogene |
| | | homolog B1) |
| CASP9(S310); | CASP9_HUMAN | Caspase-9 (CASP-9) (EC 3.4.22.62) |
| | | (Apoptotic protease Mch-6) (Apoptotic |
| | | protease-activating factor 3) (APAF-3) |
| | | (ICE-like apoptotic protease 6) (ICE-LAP6) |
| | | [Cleaved into: Caspase-9 subunit p35; |
| | | Caspase-9 subunit p10] |
| CDK13(T1058); | CDK13_HUMAN | Cyclin-dependent kinase 13 (EC |
| CDK13(S1065); | | 2.7.11.22) (EC 2.7.11.23) (CDC2-related |
| | | protein kinase 5) (Cell division cycle 2-like |
| | | protein kinase 5) (Cell division protein |
| | | kinase 13) (hCDK13) (Cholinesterase- |
| | | related cell division controller) |
| EPN1(S454); | EPN1_HUMAN | Epsin-1 (EH domain-binding mitotic |
| | | phosphoprotein) (EPS-15-interacting |
| | | protein 1) |
| GSK3A(S21); | GSK3A_HUMAN | Glycogen synthase kinase-3 alpha (GSK-3 |
| | | alpha) (EC 2.7.11.26) (Serine/threonine- |
| | | protein kinase GSK3A) (EC 2.7.11.1) |
| GYS1(S730); | GYS1_HUMAN | Glycogen [starch] synthase, muscle (EC |
| GYS1(S731); | | 2.4.1.11) |
| H2AFY(S316); | H2AY_HUMAN | Core histone macro-H2A.1 (Histone |
| | | macroH2A1) (mH2A1) (Histone H2A.y) |
| | | (H2A/y) (Medulloblastoma antigen MU- |
| | | MB-50.205) |
| JAK3(S20); | JAK3_HUMAN | Tyrosine-protein kinase JAK3 (EC |
| JAK3(T21); | | 2.7.10.2) (Janus kinase 3) (JAK-3) |
| | | (Leukocyte janus kinase) (L-JAK) |
| KDM2B(S1029); | KDM2B_HUMAN | Lysine-specific demethylase 2B (EC |
| | | 1.14.11.27) (CXXC-type zinc finger protein |
| | | 2) (F-box and leucine-rich repeat protein |
| | | 10) (F-box protein FBL10) (F-box/LRR- |
| | | repeat protein 10) (JmjC domain- |
| | | containing histone demethylation protein |
| | | 1B) (Jumonji domain-containing EMSY- |
| | | interactor methyltransferase motif |
| | | protein) (Protein JEMMA) (Protein- |
| | | containing CXXC domain 2) ([Histone-H3]- |
| | | lysine-36 demethylase 1B) |
| MLL2(S3622); | KMT2D_HUMAN | Histone-lysine N-methyltransferase 2D |
| | | (Lysine N-methyltransferase 2D) (EC |
| | | 2.1.1.364) (ALL1-related protein) |
| | | (Myeloid/lymphoid or mixed-lineage |
| | | leukemia protein 2) |
| PRKCD(S506) and/ | KPCD_HUMAN | Protein kinase C delta type (EC 2.7.11.13) |
| or PRKCD(T507); | | (Tyrosine-protein kinase PRKCD) (EC |
| | | 2.7.10.2) (nPKC-delta) [Cleaved into: |
| | | Protein kinase C delta type regulatory |
| | | subunit; Protein kinase C delta type |
| | | catalytic subunit (Sphingosine-dependent |
| | | protein kinase-1) (SDK1)] |
| CAMK1D(S179); | KCC1D_HUMAN | Calcium/calmodulin-dependent protein |
| | | kinase type 1D (EC 2.7.11.17) (CaM |
| | | kinase I delta) (CaM kinase ID) (CaM-KI |
| | | delta) (CaMKI delta) (CaMKID) (CaMKI- |
| | | like protein kinase) (CKLIK) |
| PRKCD(S302); | KPCD_HUMAN | Protein kinase C delta type (EC 2.7.11.13) |
| | | (Tyrosine-protein kinase PRKCD) (EC |
| | | 2.7.10.2) (nPKC-delta) [Cleaved into: |
| | | Protein kinase C delta type regulatory |
| | | subunit; Protein kinase C delta type |
| | | catalytic subunit (Sphingosine-dependent |
| | | protein kinase-1) (SDK1)] |
| MARCKS(S128); | MARCS_HUMAN | Myristoylated alanine-rich C-kinase |
| | | substrate (MARCKS) (Protein kinase C |
| | | substrate, 80 kDa protein, light chain) |
| | | (80K-L protein) (PKCSL) |
| MINK1(S778); | MINK1_HUMAN | Misshapen-like kinase 1 (EC 2.7.11.1) |
| | | (GCK family kinase MINK) (MAPK/ERK |
| | | kinase kinase kinase 6) (MEK kinase |
| | | kinase 6) (MEKKK 6) (Misshapen/NIK- |
| | | related kinase) (Mitogen-activated |
| | | protein kinase kinase kinase kinase 6) |
| MYC(T58); | MYC_HUMAN | Myc proto-oncogene protein (Class E |
| MYC(S64);/ | | basic helix-loop-helix protein 39) |
| MYCN(T58); | | (bHLHe39) (Proto-oncogene c-Myc) |
| MYCN(S64); | | (Transcription factor p64) |
| PSD3(S1014); | PSD3_HUMAN | PH and SEC7 domain-containing protein 3 |
| | | (Epididymis tissue protein Li 20mP) |
| | | (Exchange factor for ADP-ribosylation |
| | | factor guanine nucleotide factor 6 D) |
| | | (Exchange factor for ARF6 D) |
| | | (Hepatocellular carcinoma-associated |
| | | antigen 67) (Pleckstrin homology and |
| | | SEC7 domain-containing protein 3) |
| STAT1(S727); | STAT1_HUMAN | Signal transducer and activator of |
| | | transcription 1-alpha/beta (Transcription |
| | | factor ISGF-3 components p91/p84) |
| VIM(S339); | VIME_HUMAN | Vimentin |
| ZNF608(S627); | ZN608_HUMAN | Zinc finger protein 608 |

The invention further provides for a plurality of minimal signature panels that provide a balance between the lower number of proteins and/or phosphorylation sites comprised, versus the level of predictive ability in terms of identifying patients most responsive to midostaurin treatment. Without wishing to be bound by theory, the difference in the panels represents the multiple biochemical routes by which a patient could respond to midostaurin. Hence, the panels represent viable alternatives to identify potential responders to midostaurin treatment. In specific embodiments of the invention, these signature panels may include any one of the following:

### Panel A—POSITIVE RESPONDER:

| Phosphor- | | | |
|---|---|---|---|
| ylation | Uniprot | | Ion |
| site | ID | Protein name | Sequence |
| TP53BP1 | TP53B_ | TP53-binding | STPFIVPSSPTEQEGR |
| (T382); | HUMAN | protein 1 | [SEQ ID NO: 1] |
| | | (53BP1) | |
| | | (p53-binding | |
| | | protein 1) | |
| | | (p53BP1) | |
| MLF2 | MLF2_ | Myeloid | LAIQGPEDSPSR |
| (S240); | HUMAN | leukemia | [SEQ ID NO: 2] |
| | | factor 2 | |
| | | (Myelody- | |
| | | splasia- | |
| | | myeloid | |
| | | leukemia | |
| | | factor 2) | |
| MEFV | MEFV_ | Pyrin | SLEVTISTGEK |
| (S248); | HUMAN | (Marenostrin) | [SEQ ID NO: 3] |
| AHNAK | AHNK_ | Neuroblast | VSMPDVELNLKSPK |
| (S3426); | HUMAN | differenti- | [SEQ ID NO: 4] |
| | | ation- | |
| | | associated | |
| | | protein | |
| | | AHNAK | |
| | | (Desmoyokin) | |
| NHSL2 | NHSL2_ | NHS-like | SVSLVKDEPGLLPE |
| (S210); | HUMAN | protein 2 | GGSALPK |
| | | | [SEQ ID NO: 5] |

### Panel B—POSITIVE RESPONDER:

| Phosphory- | | | |
|---|---|---|---|
| lation | Uniprot | | |
| site | ID | Protein name | Ion Sequence |
| SYK | KSYK_ | Tyrosine- | IKSYSFPKPGHR |
| (S295); | HUMAN | protein kinase | [SEQ ID NO: 6] |
| SYK | | SYK | |
| (S297); | | (EC 2.7.10.2) | |
| | | (Spleen | |
| | | tyrosine | |
| | | kinase) | |
| | | (p72-Syk) | |
| LARP1 | LARP1_ | La-related | ESPRPLQLPGAEGP |
| (S90); | HUMAN | protein 1 (La | AISDGEEGGGEPGA |
| | | ribonucleo- | GGGAAGAAGAGR |
| | | protein | [SEQ ID NO: 7] |
| | | domain family | |
| | | member 1) | |
| SVIL | SVIL_ | Supervillin | QAHDLSPAAESSST |
| (S228); | HUMAN | (Archvillin) | FSFSGR |
| | | (p205/p250) | [SEQ ID NO: 8] |
| CCDC86 | CCD86_ | Coiled-coil | LGGLRPESPESLTS |
| (S24); | HUMAN | domain- | VSR |
| | | containing | [SEQ ID NO: 9] |
| | | protein 86 | |
| | | (Cytokine- | |
| | | induced | |
| | | protein with | |
| | | coiled-coil | |
| | | domain) | |
| PFKFB2 | F262_ | 6-phospho- | NYSVGSRPLKPLSP |
| (S486); | HUMAN | fructo-2- | LR |
| | | kinase/ | [SEQ ID NO: |
| | | fructose-2,6- | 10] |
| | | bisphosphatase | |
| | | 2 (6PF-2-K/Fru- | |
| | | 2,6-P2ase 2) | |
| | | (PFK/FBPase 2) | |
| | | (6PF-2-K/Fru- | |
| | | 2,6-P2ase heart- | |
| | | type isozyme) | |
| | | [Includes: | |
| | | 6-phospho- | |
| | | fructo-2- | |
| | | kinase | |
| | | (EC 2.7.1.105); | |
| | | Fructose-2,6- | |
| | | bisphosphatase | |
| | | (EC3.1.3.46)] | |

### Panel C—POSITIVE RESPONDER:

| Phos- | | | |
|---|---|---|---|
| phor- | Uni- | | |
| ylation | prot | | |
| site | ID | Protein name | Ion Sequence |
| SHANK3 | SHAN3_ | SH3 and multiple | SRSPSPSPLPSPA |
| (S1650); | HUMAN | ankyrin repeat | PSGGPGAPGPR |
| SHANK3 | | domains protein | [SEQ ID NO: |
| 6(S154); | | 3 (Shank3) | 11] |
| | | (Proline-rich | |
| | | synapse- | |
| | | associated | |
| | | protein 2) | |
| | | (ProSAP2) | |
| SRRM2 | SRRM2_ | Serine/arginine | SRTPLISR |
| (S1878); | HUMAN | repetitive | [SEQ ID NO: |
| SRRM2 | | matrix protein 2 | 12] |
| (T1880); | | (300 kDa | |
| | | nuclear matrix | |
| | | antigen) | |
| | | (Serine/ | |
| | | arginine-rich | |
| | | splicing factor- | |
| | | related nuclear | |
| | | matrix protein | |
| | | of 300 kDa) (SR- | |
| | | related nuclear | |
| | | matrix protein | |
| | | of 300 kDa) | |
| | | (Ser/Arg-related | |
| | | nuclear matrix | |
| | | protein of 300 | |
| | | kDa) (Splicing | |
| | | coactivator | |
| | | subunit SRm300) | |
| | | (Tax-responsive | |
| | | enhancer | |
| | | element- | |
| | | binding protein | |
| | | 803) (TaxREB803) | |
| KIAA0528 | C2CD5_ | C2 domain- | NQTYSFSPSK |
| (S297); | HUMAN | containing | [SEQ ID NO: |
| | | protein 5 (C2 | 13] |
| | | domain- | |
| | | containing | |
| | | phosphoprotein | |
| | | of 138 kDa) | |
| KIAA1429 | VIR_ | Protein | VISHDRDSPPPPP |
| (S138); | HUMAN | virilizer | PPPPPPQPQPSLK |
| | | homolog | [SEQ ID NO: |
| | | | 14] |
| MAP1A | MAP1A_ | Microtubule- | SLSPEDAESLSVL |
| (S1157); | HUMAN | associated | SVPSPDTANQEPT |
| | | protein 1A | PK |
| | | (MAP-1A) | [SEQ ID NO: |
| | | (Proliferation- | 15] |
| | | related protein | |
| | | p80) [Cleaved | |
| | | into: MAP1A | |
| | | heavy chain; | |
| | | MAP1 light | |
| | | chain LC2] | |

### Panel D—NEGATIVE RESPONDERS

| Phosphor- | Uniprot | | |
|---|---|---|---|
| ylation | | Protein | |
| site | ID | name | Ion Sequence |
| CAST | ICAL_ | Calpastatin | EGITGPPADSSKPIG |
| (T245); | HUMAN | (Calpain | PDDAIDALSSDFTCG |
| | | inhibitor) | SPTAAGK |
| | | (Sperm | [SEQ ID NO: 16] |
| | | BS-17 | |
| | | component) | |
| CBFA2T3 | MTG16_ | Protein | SSSAGPEGPQLDVPR |
| (S459); | HUMAN | CBFAT3 | [SEQ ID NO: 17] |
| | | (MTG8- | |
| | | related | |
| | | protein 2) | |
| | | (Myeloid | |
| | | translo- | |
| | | cation | |
| | | gene on | |
| | | chromosome | |
| | | 16 protein) | |
| | | (hMTG16) | |
| | | (Zinc | |
| | | finger MYND | |
| | | domain- | |
| | | containing | |
| | | protein 4) | |
| NDRG1 | NDRG1_ | Protein | SHTSEGAHLDITPNS |
| (S378); | HUMAN | NDRG1 | GAAGNSAGPK |
| | | (Differen- | [SEQ ID NO: 18] |
| | | tiation- | |
| | | related | |
| | | gene 1 | |
| | | protein) | |
| | | (DRG-1) | |
| | | (N-myc | |
| | | downstream- | |
| | | regulated | |
| | | gene | |
| | | 1 protein) | |
| | | (Nickel- | |
| | | specific | |
| | | induction | |
| | | protein | |
| | | Cap43) | |
| | | (Reducing | |
| | | agents and | |
| | | tunicamycin- | |
| | | responsive | |
| | | protein) | |
| | | (RTP) | |
| | | (Rit42) | |
| ULK1 | ULK1_ | Serine/ | ASPSPPAHAEHGGVL |
| (S477); | HUMAN | threonin | AR |
| ULK1 | | e-protein | [SEQ ID NO: 19] |
| (S479); | | kinase | |
| | | ULK1 (EC | |
| | | 2.7.11.1) | |
| | | (Autophagy- | |
| | | related | |
| | | protein | |
| | | 1 homolog) | |
| | | (ATG1) | |
| | | (hATG1) | |
| | | (Unc-51- | |
| | | like | |
| | | kinase 1) | |
| DEPTOR | DPTOR_ | DEP domain- | SPSSQETHDSPFCLR |
| (S244); | HUMAN | containing | [SEQ ID NO: 20] |
| | | mTOR- | |
| | | interacting | |
| | | protein | |
| | | (DEP domain- | |
| | | containing | |
| | | protein 6) | |

In accordance with embodiments of the present invention, the any one of above Panels A to C may be used to stratify a patient population to identify those individuals most responsive to midostaurin treatment for cancer. In a further embodiment, Panel D may be used to stratify a patient population to identify those individuals least responsive to midostaurin treatment for cancer. The Panels may be used individuals or in combination. Hence, any one or all of the Panels A to C may be used to identify a responder and cross referenced with Panel D (non-responder) to determine whether the individual is confirmed as a responder. Conversely, Panel D may be used to identify a non-responder and cross referenced with any or all of Panels A to C (responders) to determine whether the individual is confirmed as a non-responder.

It will be appreciated that the panels A to D may be used to confirm or cross reference any of the signatures for both proteomic or phosphoproteomic analysis of samples in accordance with the methods of the invention. Likewise, any one of Panels A to D may be used to cross reference or provide additional confirmatory analysis in combination with another companion diagnostic assay or test, such as a FLT3 mutation positive test.

Biomarkers shown to be particularly advantageous in both bone marrow and peripheral blood samples have the advantages of reproducibility across sample types and flexibility to be used irrespective of which sample type is most readily available in a clinical setting. The methods may comprise determining a proteomic and/or phosphoproteomic signature in a bone marrow sample or in a peripheral blood sample from the patient. Biomarkers shown to be particularly advantageous in peripheral blood samples have the advantage of use in a sample type easily obtained in relatively large quantities by a simple procedure.

Biomarkers shown to be particularly advantageous in bone marrow samples have the advantage of use in a sample type routinely obtained during cancer diagnosis, especially AML diagnosis.

The method may comprise determining in a bone marrow sample from the patient a proteomic signature comprised of the level of one or more proteins selected from the group consisting of:
Heterogeneous nuclear ribonucleoprotein M
Protein PML (E3 SUMO-protein ligase PML) (EC 2.3.2.-) (Promyelocytic leukemia
protein) (RING finger protein 71) (RING-type E3 SUMO transferase PML)
(Tripartite motif-containing protein 19) (TRIM19)
Neuroblast differentiation-associated protein AHNAK
Myoferlin
Dedicator of cytokinesis protein 10 (Zizimin-3)
Eukaryotic translation initiation factor 3 subunit D
Glutamine-fructose-6-phosphate aminotransferase
Choline-phosphate cytidylyltransferase A
V-type proton ATPase subunit B, brain isoform
Eukaryotic translation initiation factor 2 subunit 3
V-type proton ATPase catalytic subunit A

In an alternative embodiment the level of one or more proteins selected from the group consisting of:
Probable rRNA-processing protein EBP2;
Integrin alpha-5;
RNA exonuclease 4;
Glutathione S-transferase theta-2;
SAP30-binding protein;
Filensin;
Protein unc-13 homolog D;
Alpha-1,6-mannosyl-glycoprotein 2-beta-N-acetylglucosaminyltransferase;
Arfaptin-1;
Extracellular matrix protein 1;
CCA tRNA nucleotidyltransferase 1, mitochondrial;
BTB/POZ domain-containing protein 16;
Protein YIPF4;
ATP-dependent DNA helicase Q1; and
DnaJ homolog subfamily C member 2.

The biomarkers described herein may further include the proteins indicated in Table 2, each of which may be referred to by either the “full protein name” or by the corresponding entry in the “signatures” column. The “increased in” column indicates whether the biomarker is typically increased in predicted midostaurin responders or in predicted midostaurin non-responders.

As used herein, the term “midostaurin responder” typically refers to a patient who is responding or will respond to treatment with midostaurin. Likewise, the term “midostaurin non-responder” typically refers to a patient who is not responding or will not respond to treatment with midostaurin. Responding here means there is sign of clinical improvement, a cessation of clinical deterioration or a slowed rate of clinical deterioration.

TABLE 2

| UniProt ID of | Version number | | | |
|---|---|---|---|---|
| canonical sequence | and date | Full protein name | Signatures | increased in: |
| Q99848-1 | Oct. 17, 2006 - v2 | Probable rRNA-processing | EBP2_HUMAN | non-responders |
| | | protein EBP2 | | |
| P08648-1 | Oct. 10, 2002 - v2 | Integrin alpha-5 | ITA5_HUMAN | responders |
| Q9GZR2-1 | Jul. 19, 2005 - v2 | RNA exonuclease 4 | REXO4_HUMAN | non-responders |
| P0CG29-1 | Jul. 13, 2010 - v1 | Glutathione S-transferase | GST2_HUMAN | responders |
| | | theta-2 | | |
| Q9UHR5-1 | May 1, 2000 - v1 | SAP30-binding protein | S30BP_HUMAN | non-responders |
| P54136-1 | Apr. 16, 2002 - v2 | Arginine--tRNA ligase, | SYRC_HUMAN | responders |
| | | cytoplasmic | | |
| Q9H2A2-1 | Mar. 1, 2001 - v1 | 2-aminomuconic | AL8A1_HUMAN | non-responders |
| | | semialdehyde dehydrogenase | | |
| Q12934-1 | Sep. 26, 2001 - v3 | Filensin | BFSP1_HUMAN | responders |
| Q70J99-1 | Jul. 5, 2004 - v1 | Protein unc-13 homolog D | UN13D_HUMAN | responders |
| P21281-1 | Dec. 1, 2000 - v3 | V-type proton ATPase subunit | VATB2_HUMAN | responders |
| | | B, brain isoform | | |
| Q10469-1 | Oct. 1, 1996 - v1 | Alpha-1,6-mannosyl- | MGAT2_HUMAN | responders |
| | | glycoprotein 2-beta-N- | | |
| | | acetylglucosaminyltransferase | | |
| P53367-1 | May 27, 2002 - v2 | Arfaptin-1 | ARFP1_HUMAN | responders |
| P10147-1 | Jul. 1, 1989 - v1 | C-C motif chemokine 3 | CCL3_HUMAN | responders |
| Q16610-1 | Jun. 7, 2004 - v2 | Extracellular matrix protein 1 | ECM1_HUMAN | non-responders |
| Q3KQV9-1 | Mar. 18, 2008 - v2 | UDP-N-acetylhexosamine | UAP1L_HUMAN | responders |
| | | pyrophosphorylase-like | | |
| | | protein 1 | | |
| Q8IY81-1 | May 18, 2010 - v2 | pre-rRNA 2′-O-ribose RNA | SPB1_HUMAN | |
| | | methyltransferase FTSJ3 | | |
| P17275-1 | Aug. 1, 1990 - v1 | Transcription factor jun-B | JUNB_HUMAN | |
| P41091-1 | Jan. 23, 2007 - v3 | Eukaryotic translation | IF2G_HUMAN | responders |
| | | initiation factor 2 subunit 3 | | |
| P09467-1 | Nov. 2, 2010 - v5 | Fructose-1,6-bisphosphatase 1 | F16P1_HUMAN | |
| P48637-1 | Feb. 1, 1996 - v1 | Glutathione synthetase | GSHB_HUMAN | responders |
| P00813-1 | Jan. 23, 2007 - v3 | Adenosine deaminase | ADA_HUMAN | |
| P38606-1 | Aug. 2, 2002 - v2 | V-type proton ATPase | VATA_HUMAN | responders |
| | | catalytic subunit A | | |
| Q96Q11-1 | May 18, 2010 - v2 | CCA tRNA | TRNT1_HUMAN | non-responders |
| | | nucleotidyltransferase 1, | | |
| | | mitochondrial | | |
| P05023-1 | Aug. 13, 1987 - v1 | Sodium/potassium- | AT1A1_HUMAN | responders |
| | | transporting ATPase subunit | | |
| | | alpha-1 | | |
| O60603-1 | Aug. 1, 1998 - v1 | Toll-like receptor 2 | TLR2_HUMAN | |
| Q32M84-1 | Jul. 25, 2006 - v2 | BTB/POZ domain-containing | BTBDG_HUMAN | non-responders |
| | | protein 16 | | |
| P25490-1 | Dec. 15, 1998 - v2 | Transcriptional repressor | TYY1_HUMAN | |
| | | protein YY1 | | |
| O15347-1 | Jan. 23, 2007 - v4 | High mobility group protein | HMGB3_HUMAN | |
| | | B3 | | |
| Q9UJF2-1 | May 4, 2001 - v2 | Ras GTPase-activating protein | NGAP_HUMAN | |
| | | nGAP | | |
| O60229-1 | Jul. 31, 2019 - v3 | Kalirin | KALRN_HUMAN | non-responders |
| Q9Y5S2-1 | Apr. 4, 2006 - v2 | Serine/threonine-protein | MRCKB_HUMAN | responders |
| | | kinase MRCK beta | | |
| Q9H6W3-1 | Jan. 19, 2010 - v2 | Ribosomal oxygenase 1 | RIOX1_HUMAN | non-responders |
| Q96SU4-1 | Feb. 11, 2002 - v2 | Oxysterol-binding protein- | OSBL9_HUMAN | non-responders |
| | | related protein 9 | | |
| Q9BSR8-1 | Jun. 1, 2001 - v1 | Protein YIPF4 | YIPF4_HUMAN | responders |
| P46063-1 | Dec. 16, 2008 - v3 | ATP-dependent DNA helicase | RECQ1_HUMAN | non-responders |
| | | Q1 | | |
| Q99543-1 | May 18, 2010 - v4 | DnaJ homolog subfamily C | DNJC2_HUMAN | responders |
| | | member 2 | | |
| Q86Y82-1 | Jun. 1, 2003 - v1 | Syntaxin-12 | STX12_HUMAN | non-responders |
| Q9Y316-1 | Nov. 1, 1999 - v1 | Protein MEMO1 | MEMO1_HUMAN | non-responders |
| Q96LW7-1 | Dec. 1, 2001 - v1 | Caspase recruitment domain- | CAR19_HUMAN | non-responders |
| | | containing protein 19 | | |
| P0DOX8-1 | Mar. 15, 2017 - v1 | Immunoglobulin lambda-1 | IGL1_HUMAN | responders |
| | | light chain | | |
| Q8IU85-1 | Mar. 1, 2003 - v1 | Calcium/calmodulin- | KCC1D_HUMAN | non-responders |
| | | dependent protein kinase | | |
| | | type 1D | | |
| Q8TB40-1 | Jun. 1, 2002 - v1 | (Lyso)-N- | ABHD4_HUMAN | responders |
| | | acylphosphatidylethanolamine | | |
| | | lipase | | |
| Q14692-1 | Nov. 1, 1996 - v1 | Ribosome biogenesis protein | BMS1_HUMAN | responders |
| | | BMS1 homolog | | |
| Q9UNA1-1 | May 1, 2000 - v1 | Rho GTPase-activating | RHG26_HUMAN | responders |
| | | protein 26 | | |
| Q8IY17-4 | Sep. 12, 2018 - v3 | Patatin-like phospholipase | PLPL6_HUMAN | responders |
| | | domain-containing protein 6 | | |
| Q86SQ6-3 | Nov. 30, 2010 - v3 | Adhesion G protein-coupled | AGRA1_HUMAN | non-responders |
| | | receptor A1 | | |
| Q01432-1 | Jul. 1, 1993 - v1 | AMP deaminase 3 | AMPD3_HUMAN | non-responders |

The biomarkers described herein include the phosphorylation sites indicated in Table 3, each of which may be referred to by either the “full phosphorylation site name” or by the corresponding entry in the “signatures” column. The “increased in” column indicates whether the biomarker is typically increased in predicted midostaurin responders or in predicted midostaurin non-responders. As used herein, the residue numbering of the phosphorylation site(s) corresponds to the residue numbering in the UniProt ID of the canonical sequence with the version number and date indicated. All protein sequences start from the methionine 1 position for each protein listed. For each phosphorylation site /signature a number of “Peptide with alternative phosphorylation sites” are given; all possible combinations of phosphorylation sites embraced by the “Peptide with alternative phosphorylation sites” column are explicitly contemplated herein. Accordingly, any reference to a phosphorylation site or signature may be replaced by a reference to the corresponding entry in the “Peptide with alternative phosphorylation sites” column, or any one or more of the phosphorylation sites embraced “Peptide with alternative phosphorylation sites” column. Without being bound by theory, the phosphorylation site given in the “full phosphorylation site name” column is the preferred phosphorylation site of the phosphorylation sites embraced “Peptide with alternative phosphorylation sites” column. Further details on the peptides and phosphorylation sites referred to in the “Peptide with alternative phosphorylation sites” are given in Table 4, wherein the “Gene” column refers to the name of a gene also provided in the “Signatures” column of Table 3, wherein the UniProt ID of the canonical sequence, version number and date and name of the protein (as given in the “fully phosphorylation site name” column of Table 2, albeit there for only a single phosphorylation site) correspond to those in Table 3 Biomarkers according to the invention include any peptide of Table 4 phosphorylated at any one of the residues belonging to that peptide recited in Table 4. Biomarkers according to the invention include any peptide of Table 4 phosphorylated at any two of the residues belonging to that peptide recited in Table 4. Biomarkers according to the invention include any peptide of Table 4 phosphorylated at any three of the residues belonging to that peptide recited in Table 4. Biomarkers according to the invention may include any one of the phosphorylation sites recited in Table 5, wherein the “Signatures” column refers to the name of a gene also provided in the “Signatures” column of Table 3, wherein the UniProt ID of the canonical sequence, version number and date and name of the protein (as given in the “fully phosphorylation site name” column of Table 3, albeit there for only a single phosphorylation site) correspond to those in Table 3. Biomarkers according to the invention include any peptide of Table 5 phosphorylated at any one of the residues belonging to that peptide recited in Table 5. Biomarkers according to the invention include any peptide of Table 5 phosphorylated at any two of the residues belonging to that peptide recited in Table 5. Biomarkers according to the invention include any peptide of Table 5 phosphorylated at any three of the residues belonging to that peptide recited in Table 5.

TABLE 3

| UniProt ID | | Full | | | |
|---|---|---|---|---|---|
| of canonical | Version number | phosphorylation | Peptide with alternative | | increased |
| sequence | and date | site name | phosphorylation sites | Signatures | in: |
| P42224-1 | Nov. 1, 1997 - v2 | T719 of Signal transducer and | 717 to 736 from human STAT1, that are single, | STAT1 | responders |
| | | activator of transcription 1- | double or triple phosphorylated on any of the | (T719) | |
| | | alpha/beta; | Serine [S735] or 2 Threonine [T719, T720] | | |
| | | | residues. | | |
| P13807-1 | Feb. 1, 1996 - v2 | T729 and/or S730 of | 708 to 737 from human GYS1, that are single, | GYS1 | responders |
| | | Glycogen [starch] synthase, | double or triple phosphorylated on any of the 7 | (T729)(S730) | |
| | | muscle; | Serine [S710, S723, S729, S716, S717, S718, S720] | | |
| | | | or 5 Threonine [T721, T731, T730, T713, T715] | | |
| | | | residues. | | |
| Q9ULD9-1 | May 18, 2010 - v4 | S627 of Zinc finger protein | | ZNF608 | non- |
| | | 608; | | (S627) | responders |
| P06730-1 | Feb. 1, 1996 - v2 | T77 of Eukaryotic translation | 64 to 99 from human EIF4EBP1, that are single, | EIF4EBP1 | responders |
| | | initiation factor 4E-binding | double or triple phosphorylated on any of the 6 | (T77) | |
| | | protein 1; | Serine [S65, S94, S68, S86, S85, S96] or 4 Threonine | | |
| | | | [T70, T83, T77, T82] residues. | | |
| P06730-1 | Feb. 1, 1996 - v2 | T46 of Eukaryotic translation | 21 to 51 from human EIF4EBP2, that are single, | EIF4EBP2 | responders |
| | | initiation factor 4E-binding | double or triple phosphorylated on any of the 1 | (T46) | |
| | | protein 2; | Serine [S25] or 7 Threonine [T46, T37, T36, T44, | | |
| | | | T41, T45, T50] or Tyrosine [Y34] residues. | | |
| Q05655-1 | Sep. 2, 2008 - v2 | S302 of Protein kinase C | 301 to 318 from human PRKCD, that are single, | PRKCD | responders |
| | | delta type; | double or triple phosphorylated on any of the 4 | (S302) | |
| | | | Serine [S304, S306, S302, S307] or 1 Tyrosine | | |
| | | | [Y313] residues. | | |
| Q96B36-1 | Dec. 1, 2001 - v1 | S183 of Proline-rich AKT1 | 183 to 194 from human AKT1S1, that are single, | AKT1S1 | responders |
| | | substrate 1; | double or triple phosphorylated on any of the 2 | (S183) | |
| | | | Serine [S183, S187] residues. | | |
| P49840-1 | Dec. 1, 2000 - v2 | T19 of Glycogen synthase | 17 to 50 from human GSK3A, that are single, | GSK3A (T19) | responders |
| | | kinase-3 alpha; | double or triple phosphorylated on any of the 4 | | |
| | | | Serine [S21, S20, S39, S41] or 2 Threonine [T19, | | |
| | | | T46] residues. | | |
| Q05655-1 | Sep. 2, 2008 - v2 | Y313 of Protein kinase C | 301 to 318 from human PRKCD, that are single, | PRKCD | responders |
| | | delta type | double or triple phosphorylated on any of the 4 | (Y313) | |
| | | | Serine [S304, S306, S302, S307] or 1 Tyrosine | | |
| | | | [Y313] residues. | | |
| P01106-1 | Aug. 13, 1987 - v1 | T58 of Myc proto-oncogene | 52 to 65 from human MYC or MYCN, that are | MYC (T58) | non- |
| | | protein and/or T58 of N-myc | single, double or triple phosphorylated on any of | MYCN (T58) | responders |
| | | proto-oncogene protein; | the 2 Serine [S62, S64] or 1 Threonine [T58] | | |
| | | | residues. | | |
| P01106-1 | Aug. 13, 1987 - v1 | T58 of Myc proto-oncogene | 52 to 65 from human MYC, that are single, double | MYC (T58) | non- |
| | | protein | or triple phosphorylated on any of the 2 Serine | | responders |
| | | | [S62, S64] or 1 Threonine [T58] residues. | | |
| O75367-1 | Jan. 23, 2007 - v4 | S316 of Core histone macro- | 308 to 318 from human H2AFY, that are single, | H2AFY | non- |
| | | H2A.1; | double or triple phosphorylated on any of the 3 | (S316) | responders |
| | | | Serine [S308, S313, S316] residues. | | |
| Q8NHM5-1 | Oct. 1, 2002 - v1 | S1029 of Lysine-specific | 1022 to 1034 from human KDM2B, that are single, | KDM2B | non- |
| | | demethylase 2B; | double or triple phosphorylated on any of the 2 | (S1029) | responders |
| | | | Serine [S1029, S1031] residues. | | |
| O14686-1 | Nov. 30, 2010 - v2 | S3620 of Histone-lysine N- | 3599 to 3626 from human KMT2D, that are single, | KMT2D | non- |
| | | methyltransferase 2D; | double or triple phosphorylated on any of the 2 | (S3620) | responders |
| | | | Serine [S3620, S3624] residues. | | |
| P15056-1 | Jul. 19, 2004 - v4 | S363 of Serine/threonine- | 363 to 384 from human BRAF, that are single, | BRAF (S363) | responders |
| | | protein kinase B-raf; | double or triple phosphorylated on any of the 3 | | |
| | | | Serine [S365, S363, S364] or 1 Threonine [T373] | | |
| | | | residues. | | |
| P52333-1 | Jul. 19, 2004 - v2 | S15 and/or S17 of Tyrosine- | 15 to 33 from human JAK3, that are single, double | JAK3 (S15) | non- |
| | | protein kinase JAK3; | or triple phosphorylated on any of the 3 Serine | (S17) | responders |
| | | | [S17, S15, S20] or 1 Threonine [T21] residues. | | |
| Q13315-1 | Jan. 22, 2014 - v4 | S2996 of Serine-protein | 2994 to 3004 from human ATM, that are single, | ATM (S2996) | responders |
| | | kinase ATM; | double or triple phosphorylated on any of the 1 | | |
| | | | Serine [S2996] residues. | | |
| P08670-1 | Jan. 23, 2007 - v4 | T327 of Vimentin; | 322 to 334 from human VIM, that are single, | VIM (T327) | responders |
| | | | double or triple phosphorylated on any of the 1 | | |
| | | | Serine [S325] or 1 Threonine [T327] residues; or | | |
| | | | 322 to 342 from human VIM, that are single, | | |
| | | | double or triple phosphorylated on any of the 2 | | |
| | | | Serine [S325, S339] or 2 Threonine [T327, T336] | | |
| | | | residues. | | |
| Q9NYI0-1 | Nov. 13, 2007 - v2 | S1009 of PH and SEC7 | 1009 to 1025 from human PSD3, that are single, | PSD3 | non- |
| | | domain-containing protein | double or triple phosphorylated on any of the 5 | (S1009) | responders |
| | | 3; | Serine [S1009, S1011, S1012, S1014, S1020] or 2 | | |
| | | | Threonine [T1019, T1023] residues. | | |
| Q9Y6I3-2 | May 3, 2011 - v2 | S447 of Epsin-1; | 446 to 469 from human EPN1, that are single, | EPN1 (S447) | responders |
| | | | double or triple phosphorylated on any of the 2 | | |
| | | | Serine [S447, S454] or 4 Threonine [T460, T462, | | |
| | | | T464, T467] residues. | | |
| P55211-1 | Feb. 22, 2003 - v3 | S310 of Caspase-9; | 293 to 324 from human CASP9, that are single, | CASP9 | responders |
| | | | double or triple phosphorylated on any of the 3 | (S310) | |
| | | | Serine [S310, S307, S302] or 1 Threonine [T301] | | |
| | | | residues. | | |
| P29966-1 | Jan. 23, 2007 - v4 | T120 of Myristoylated | 88 to 137 from human MARCKS, that are single, | MARCKS | responders |
| | | alanine-rich C-kinase | double or triple phosphorylated on any of the | (T120) | |
| | | substrate; | Serine [S128] or Threonine [T120] residues. | | |
| Q14004-1 | May 10, 2005 - v2 | S1054 of Cyclin-dependent | 1045 to 1069 from human CDK13, that are single, | CDK13 | non- |
| | | kinase 13; | double or triple phosphorylated on any of the 4 | (S1054) | responders |
| | | | Serine [S1048, S1054, S1065, S1066] or 2 | | |
| | | | Threonine [T1056, T1058] residues. | | |
| Q8TB45-1 | Jul. 7, 2009 - v2 | S244 of DEP domain- | 235 to 249 from human DEPTOR, that are single, | DEPTOR | non- |
| | | containing mTOR-interacting | double or triple phosphorylated on any of the 4 | (S244) | responders |
| | | protein; | Serine [S244, S235, S237, S238] or 1 Threonine | | |
| | | | [T241] residues. | | |
| Q8N4C8-1 | May 18, 2010 - v2 | S772 of Misshapen-like | 769 to 786 from human MINK1, that are single, | MINK1 | non- |
| | | kinase 1; | double or triple phosphorylated on any of the 5 | (S772) | responders |
| | | | Serine [S772, S773, S777, S778, S782] residues. | | |
| Q8IU85-1 | Mar. 1, 2003 - v1 | S179 of Calcium/calmodulin- | 175 to 200 from human CAMK1D, that are single, | CAMK1D | non- |
| | | dependent protein kinase | double or triple phosphorylated on any of the | (S179) | responders |
| | | type 1D; | Serine [S179], Threonine [T180] or Tyrosine | | |
| | | | [Y187] residues. | | |
| P42229-1 | Nov. 1, 1995 - v1 | S780 of Signal transducer and | | STAT5A | non- |
| | | activator of transcription 5A | | (S780) | responders |
| Q05655-1 | Sep. 2, 2008 - v2 | S506 of Protein kinase C delta | 301 to 318 from human PRKCD, that are single, | PRKCD | non- |
| | | type | double or triple phosphorylated on any of the | (S506) | responders |
| | | | 4 Serine [S304, S306, S302, S307] or 1 Tyrosine | | |
| | | | [Y313] residues. | | |

TABLE 4

| Gene | pep_start | pep_end | Serine_residues | Threonine_residues | Tyrosine_residues |
|---|---|---|---|---|---|
| AKT1S1 | 183 | 194 | [S183, S187] | | |
| DEPTOR | 235 | 249 | [S244, S235, S237, | [T241] | |
| | | | S238] | | |
| EIF4EBP1 | 64 | 99 | [S65, S94, S68, S86, | [T70, T83, T77, T82] | |
| | | | S85, S96] | | |
| EIF4EBP2 | 21 | 51 | [S25] | [T46, T37, T36, T44, | [Y34] |
| | | | | T41, T45, T50] | |
| BRAF | 361 | 384 | [S365, S363, S364] | [T373] | |
| CASP9 | 293 | 324 | [S310, S307, S302] | [T301] | |
| GSK3A | 17 | 50 | [S21, S20, S39, S41] | [T19, T46] | |
| GYS1 | 708 | 737 | [S710, S723, S729, | [T721, T731, T730, | |
| | | | S716, S717, S720, | T713, T715] | |
| | | | S718] | | |
| MYC | 52 | 66 | [S62, S64] | [T58] | |
| MYCN | 52 | 65 | [S62, S64] | [T58] | |
| JAK3 | 15 | 33 | [S17, S15, S20] | [T21] | |
| STAT1 | 717 | 736 | [S735] | [T719, T720] | |
| PRKCD | 301 | 318 | [S304, S306, S302, | | [Y313] |
| | | | S307] | | |
| MARCKS | 88 | 137 | [S128] | [T120] | |
| CAMK1D | 175 | 200 | [S179] | [T180] | [Y187] |
| MINK1 | 769 | 786 | [S772, S773, S777, | | |
| | | | S778, S782] | | |
| ATM | 2994 | 3004 | [S2996] | | |
| EPN1 | 446 | 469 | [S447, S454] | [T460, T462, T464, | |
| | | | | T467] | |
| H2AFY | 308 | 318 | [S308, S313, S316] | | |
| KDM2B | 1022 | 1034 | [S1029, S1031] | | |
| KMT2D | 3599 | 3626 | [S3620, S3624] | | |
| CDK13 | 1045 | 1069 | [S1048, S1054, | [T1056, T1058] | |
| | | | S1065, S1066] | | |
| PSD3 | 1009 | 1025 | [S1009, S1011, | [T1019, T1023] | |
| | | | S1012, S1014, | | |
| | | | S1020] | | |
| VIM | 322 | 342 | [S325, S339] | [T327, T336] | |

TABLE 5
Signatures

PRKCD (S304)
PRKCD (S306)
PRKCD (S307)
AKT1S1 (S183)
AKT1S1 (S187)
DEPTOR (S235)
DEPTOR (S237)
DEPTOR (S238)
DEPTOR (S244)
DEPTOR (T241)
EIF4EBP1 (S85)
EIF4EBP1 (S86)
EIF4EBP1 (S94)
EIF4EBP1 (S96)
EIF4EBP1 (T77)
EIF4EBP1 (T82)
EIF4EBP1 (T83)
EIF4EBP2 (S25)
EIF4EBP2 (T36)
EIF4EBP2 (T41)
EIF4EBP2 (T44)
EIF4EBP2 (T45)
EIF4EBP2 (T46)
EIF4EBP2 (T50)
EIF4EBP2 (Y34)
EIF4EBP2 (Y37)
BRAF (S363)
BRAF (S364)
BRAF (S365)
BRAF (T373)
CASP9 (S302)
CASP9 (S307)
CASP9 (S310)
CASP9 (T301)
GSK3A (S20)
GSK3A (S39)
GSK3A (S41)
GSK3A (T46)
GYS1 (S710)
GYS1 (S716)
GYS1 (S717)
GYS1 (S718)
GYS1 (S720)
GYS1 (S723)
GYS1 (S729)
GYS1 (T713)
GYS1 (T715)
GYS1 (T721)
GYS1 (T730)
GYS1 (T731)
MYC (S62)
MYC (S64)
MYC (T58)
MYC or MYCN (S62)
MYC or MYCN (S64)
MYC or MYCN (T58)
JAK3 (S15)
JAK3 (S17)
JAK3 (S20)
JAK3 (T21)
CAMK1D (S179)
CAMK1D (T180)
CAMK1D (Y187)

In a specific embodiment the phosphorylation signature may comprise phosphorylation sites indicated in Table 6, each of which may be referred to by the full “phosphorylation site” name. The “biomarker group” column indicates that the biomarker is particularly suited to use in combination with one of the panels A to D described above. Hence, each of the biomarkers in Table 6 may be used to expand one or more of the pales A to D to increase the predictive capacity of that panel accordingly to identify predicted midostaurin responders or predicted midostaurin non-responders.

TABLE 6

| bio | Phosphor - | | | biomarker_ |
|---|---|---|---|---|
| marker_ | ylation | Uniprot | | group - |
| Panel | Site | ID | Protein name | protein ion |
| Panel B | BPTF | BPTF_ | Nucleosome-remodeling factor subunit BPTF | QEQSPNANNDQPEDLI |
| | (S1251); | HUMAN | (Bromodomain and PHD finger-containing | QGCSESDSSVLR |
| | | | transcription factor) (Fetal Alz-50 clone | [SEQ ID NO: 21] |
| | | | 1 protein) (Fetal Alzheimer antigen) | |
| Panel A | MAPKBP1 | MABP1_ | Mitogen-activated protein kinase-binding | SISVGENLGLVAEPQA |
| | (S1263); | HUMAN | protein 1 (JNK-binding protein 1) (JNKBP- | HAPIR |
| | | | 1) | [SEQ ID NO: 22] |
| Panel A | REEP4 | REEP4_ | Receptor expression-enhancing protein 4 | SFSMQDLR |
| | (S152); | HUMAN | | [SEQ ID NO: 23] |
| Panel A | ZNF408 | ZN408_ | Zinc finger protein 408 (PR domain zinc | AKTPEPGAQQSGFPTL |
| | (T322); | HUMAN | finger protein 17) | SR |
| | | | | [SEQ ID NO: 24] |
| Panel C | EFR3A | EFR3A_ | Protein EFR3 homolog A (Protein EFR3- | IGPPSSPSATDKEENP |
| | (T224); | HUMAN | like) | AVLAENCFR |
| | | | | [SEQ ID NO: 25] |
| Panel C | SRRM2 | SRRM2_ | Serine/arginine repetitive matrix protein | SRSPLAIR |
| | (S2044); | HUMAN | 2 (300 kDa nuclear matrix antigen) | [SEQ ID NO: 26] |
| | SRRM2 | | (Serine/arginine-rich splicing factor- | |
| | (S2046); | | related nuclear matrix protein of 300 | |
| | | | kDa) (SR-related nuclear matrix protein | |
| | | | of 300 kDa) (Ser/Arg-related nuclear | |
| | | | matrix protein of 300 kDa) (Splicing | |
| | | | coactivator subunit SRm300) (Tax- | |
| | | | responsive enhancer element-binding | |
| | | | protein 803) (TaxREB803) | |
| Panel A | IRAK3 | IRAK3_ | Interleukin-1 receptor-associated kinase | AIHUITNYGAVLSPSE |
| | (S112); | HUMAN | 3 (IRAK-3) (IL-1 receptor-associated | K |
| | | | kinase M) (IRAK-M) (Inactive IL-1 | [SEQ ID NO: 27] |
| | | | receptor-associated kinase 3) | |
| Panel A | PPP1R9B | NEB2_ | Neurabin-2 (Neurabin-II) (Protein phospha- | ASSLNENVDHSALLK |
| | (S100); | HUMAN | tase 1 regulatory subunit 9B) (Spinophilin) | [SEQ ID NO: 28] |
| Panel A | SGK223 | PRAG1_ | Inactive tyrosine-protein kinase PRAG1 | SASFAFEFPK |
| | (S694); | HUMAN | (PEAK1-related kinase-activating pseudo- | [SEQ ID NO: 29] |
| | | | kinase 1) (Pragmin) (Sugen kinase 223) | |
| | | | (SgK223) | |
| Panel A | LILRB3 | LIRB3_ | Leukocyte immunoglobulin-like receptor | RSSPAADVQEENLYAA |
| | (S503); | HUMAN | subfamily B member 3 (LIR-3) (Leukocyte | VK |
| | | | immunoglobulin-like receptor 3) (CD85 | [SEQ ID NO: 30] |
| | | | antigen-like family member A) (Immuno- | |
| | | | globulin-like transcript 5) (ILT-5) | |
| | | | (Monocyte inhibitory receptor HL9) | |
| | | | (CD antigen CD85a) | |
| Panel A | HIDE1 | HIDE1_ | Protein HIDE1 | KRPTSTSSSPETPEFS |
| | (S217); | HUMAN | | TFR |
| | HIDE1 | | | [SEQ ID NO: 31] |
| | (T220); | | | |
| Panel A | SRRM1 | SRRM1_ | Serine/arginine repetitive matrix protein | EKTPELPEPSVK |
| | (T220); | HUMAN | 1 (SR-related nuclear matrix protein of | [SEQ ID NO: 32] |
| | | | 160 kDa) (SRm160) (Ser/Arg-related | |
| | | | nuclear matrix protein) | |
| Panel A | BRAT1 | BRAT1_ | BRCA1-associated ATM activator 1 (BRCA1- | GSPNTASAEATLPR |
| | (S747); | HUMAN | associated protein required for ATM | [SEQ ID NO: 33] |
| | | | activation protein 1) | |
| Panel A | TLE3 | TLE3_ | Transducin-like enhancer protein 3 | DAPTSPASVASSSSTP |
| | (S293); | HUMAN | (Enhancer of split groucho-like protein | SSK |
| | | | 3) (ESG3) | [SEQ ID NO: 34] |
| Panel C | EGLN1 | EGLN1_ | Egl nine homolog 1 (EC 1.14.11.29) | AKPPADPAAAASPCR |
| | (S125); | HUMAN | (Hypoxia-inducible factor prolyl | [SEQ ID NO: 35] |
| | | | hydroxylase 2) (HIF-PH2) (HIF-prolyl | |
| | | | hydroxylase 2) (HPH-2) | |
| | | | (Prolyl hydroxylase domain-containing | |
| | | | protein 2) (PHD2) (SM-20) | |
| Panel A | NDRG1 | NDRG1_ | Protein NDRG1 (Differentiation-related gene | SRTASGSSVTSLDGTR |
| | (S333); | HUMAN | 1 protein) (DRG-1) (N-myc downstream- | [SEQ ID NO: 36] |
| | NDRG1 | | regulated gene 1 protein) (Nickel-specific | |
| | (T335); | | induction protein Cap43) (Reducing agents | |
| | | | and tunicamycin-responsive protein) | |
| | | | (RTP) (Rit42) | |
| Panel A | NUMB | NUMB_ | Protein numb homolog (h-Numb) (Protein | IVVGSSVAPGNTAPSP |
| | (T251); | HUMAN | S171) | SSPTSPTSDATTSLEM |
| | NUMB | | | NNPHAIPR |
| | (S252); | | | [SEQ ID NO: 37] |
| Panel C | SEC61B | SC61B_ | Protein transport protein Sec61 subunit | PGPTPSGTNVGSSGRS |
| | (S19); | HUMAN | beta | PSK |
| | | | | [SEQ ID NO: 39] |
| Panel A | TFPT | TFPT_ | TCF3 fusion partner (INO80 complex subunit | LLPYPTLASPASD |
| | (S252); | HUMAN | F) (Protein FB1) | [SEQ ID NO: 40] |
| Panel A | CCDC86 | CCD86_ | Colled-coil domain-containing protein 86 | ALVEFESNPEETREPG |
| | (S50); | HUMAN | (Cytokine-induced protein with colled-coil | SPPSVQR |
| | | | domain) | [SEQ ID NO: 41] |
| Panel A | ZMYM3 | ZMYM3_ | Zinc finger MYM-type protein 3 (Zinc finger | TGSPGPELLFHEGQQK |
| | (S464); | HUMAN | protein 261) | [SEQ ID NO: 42] |
| Panel A | TLK2 | TLK2_ | Serine/threonine-protein kinase tousled- | KSVSTSSPAGAAIAST |
| | (S771); | HUMAN | like 2 (EC 2.7.11.1) (HsHPK) (PKU-alpha) | SGASNNSSSN |
| | | | (Tousled-like kinase 2) | [SEQ ID NO: 43] |
| Panel B | SEPT6 | SEPT6_ | Septin-6 | TAAELLQSQGSQAGGS |
| | (T418); | HUMAN | | QTLKR |
| | | | | [SEQ ID NO: 44] |
| Panel C | IL1RAP | IL1AP_ | Interleukin-1 receptor accessory protein | RSSSDEQGLSYSSLK |
| | (S557); | HUMAN | (IL-1 receptor accessory protein) (IL-1RAcP) | [SEQ ID NO: 45] |
| | IL1RAP | | (EC 3.2.2.6) (Interleukin-1 receptor 3) (IL- | |
| | (S563); | | 1R-3) (IL-1R3) | |
| Panel A | PELP1 | PELP1_ | Proline-, glutamic acid- and leucine-rich | AGSNEDPILAPSGTPP |
| | (T749); | HUMAN | protein 1 (Modulator of non-genomic activity | PTIPPDETFGGR |
| | | | of estrogen receptor) (Transcription factor | [SEQ ID NO: 46] |
| | | | HMX3) | |
| Panel B | DUS3L | DUS3L_ | tRNA-dihydrouridine(47) synthase [NAD(P) | QENCGAQQVPAGPGTS |
| | (S276); | HUMAN | (+)]-like (EC 1.3.1.-) (tRNA- | TPPSSPVR |
| | DUS3L | | dihydrouridine synthase 3-like) | [SEQ ID NO: 47] |
| | (S277); | | | |
| Panel A | EVI2B | EVI2B_ | Protein EVI2B (Ecotropic viral integration | RTSIISLTPWKPSK |
| | (T273); | HUMAN | site 2B protein homolog) (EVI-2B) (CD | [SEQ ID NO: 48] |
| | | | antigen CD361) | |
| Panel A | DFFA | DFFA_ | DNA fragmentation factor subunit alpha | SISASKASPPGDLQNP |
| | (S315); | HUMAN | (DNA fragmentation factor 45 kDa subunit) | K |
| | | | (DFF-45) (Inhibitor of CAD) (ICAD) | [SEQ ID NO: 49] |
| Panel B | DOCK8 | DOCK8_ | Dedicator of cytokinesis protein 8 | VMSSSNPDLAGTHSAA |
| | (S913); | HUMAN | | DEEVK |
| | | | | [SEQ ID NO: 50] |
| Panel C | YEATS2 | YETS2_ | YEATS domain-containing protein 2 | ISTASQVSQGTGSPVP |
| | (S536); | HUMAN | | K |
| | | | | [SEQ ID NO: 50] |
| Panel A | GSE1 | GSE1_ | Genetic suppressor element 1 | ALSAAVADSLTNSPR |
| | (S909); | HUMAN | | [SEQ ID NO: 51] |
| Panel A | GAB2 | GAB2_ | GRB2-associated-binding protein 2 (GRB2- | SSPAELSSSSQHLLR |
| | (S147); | HUMAN | associated binder 2) (Growth factor | [SEQ ID NO: 52] |
| | | | receptor bound protein 2-associated | |
| | | | protein 2) (pp100) | |
| Panel A | CHAMP1 | CHAP1_ | Chromosome alignment-maintaining phospho- | LAPVPSPEPQKPAPVS |
| | (S204); | HUMAN | protein 1 (Zinc finger protein 828) | PESVK |
| | | | | [SEQ ID NO: 53] |
| Panel C | CRKL | CRKL_ | Crk-like protein | YPSPPMGSVSAPNLPT |
| | (T120); | HUMAN | | AEDNLEYVR |
| | | | | [SEQ ID NO: 54] |
| Panel B | TOP2B | TOP2B_ | DNA topoisomerase 2-beta (EC 5.6.2.2) | SEDDSAKFDSNEEDSA |
| | (S1478); | HUMAN | (DNA topoisomerase Il, beta isozyme) | SVFSPSFGLK |
| | | | | [SEQ ID NO: 55] |
| Panel B | EPS15 | EPS15_ | Epidermal growth factor receptor substrate | QSTDPFATSSTDPFSA |
| | (S681); | HUMAN | 15 (Protein Eps15) (Protein AF-1p) | ANNSSITSVETLK |
| | | | | [SEQ ID NO: 56] |
| Panel A | PDS5A | PDS5A_ | Sister chromatid cohesion protein PDS5 | AAVGQESPGGLEAGNA |
| | (S1305); | HUMAN | homolog A (Cell proliferation-inducing | K |
| | | | gene 54 protein) (Sister chromatid | [SEQ ID NO: 57] |
| | | | cohesion protein 112) (SCC-112) | |
| Panel A | AHNAK | AHNK_ | Neuroblast differentiation-associated | FKAEAPLPSPK |
| | (S5110); | HUMAN | protein AHNAK (Desmoyokin) | [SEQ ID NO: 58] |
| Panel A | FAM129B | NIBA2_ | Protein Niban 2 (Meg-3) (Melanoma | AAPEASSPPASPLQHL |
| | (S696); | HUMAN | invasion by ERK) (MINERVA) (Niban-like | LPGK |
| | | | protein 1) (Protein FAM129B) | [SEQ ID NO: 59] |
| Panel A | TTC33 | TTC33_ | Tetratricopeptide repeat protein 33 (TPR | SEAPAEVTHFSPK |
| | (S197); | HUMAN | repeat protein 33) (Osmosis-responsive | [SEQ ID NO: 60] |
| | | | factor) | |
| Panel C | RANBP2 | RBP2_ | E3 SUMO-protein ligase RanBP2 (EC | ELVGPPLAETVFTPKT |
| | (T1396); | HUMAN | 2.3.2.-) (358 kDa nucleoporin) (Nuclear | SPENVQDR |
| | RANBP2 | | pore complex protein Nup358) (Nucleoporin | [SEQ ID NO: 61] |
| | (S1400); | | Nup358) (Ran-binding protein 2) (RanBP2) | |
| | | | (p270) | |
| Panel A | AHNAK | AHNK_ | Neuroblast differentiation-associated | KGDRSPEPGQTWTR |
| | (S93); | HUMAN | protein AHNAK (Desmoyokin) | [SEQ ID NO: 62] |
| Panel C | ENO1 | ENOG_ | Gamma-enolase (EC 4.2.1.11) (2-phospho-D- | AAVPSGASTGIYEALE |
| | (Y44); | HUMAN | glycerate hydro-lyase) (Enolase 2) | LR |
| | ENO3 | | (Neural enolase) (Neuron-specific enolase) | [SEQ ID NO: 63] |
| | (Y44); | | (NSE) | |
| | ENO2 | | | |
| | (Y44); | | | |
| Panel A | RCC1 | RCC1_ | Regulator of chromosome condensation (Cell | RRSPPADAIPK |
| | (S11); | HUMAN | cycle regulatory protein) (Chromosome | [SEQ ID NO: 64] |
| | | | condensation protein 1) | |
| Panel B | TLN1 | TLN1_ | Talin-1 | ASVPTIQDQASAMQLS |
| | (S1021); | HUMAN | | QCAK |
| | | | | [SEQ ID NO: 65] |
| Panel A | ZNF768 | ZN768_ | Zinc finger protein 768 | FEPESPGFESR |
| | (S83); | HUMAN | | [SEQ ID NO: 66] |
| Panel B | PPHLN1 | PPHLN_ | Periphilin-1 (CDC7 expression repressor) | ERPVQSLKTSRDTSPS |
| | (T204); | HUMAN | (CR) (Gastric cancer antigen Ga50) | SGSAVSSSK |
| | PPHLN1 | | | [SEQ ID NO: 67] |
| | (S205); | | | |
| Panel A | AHNAK | AHNK_ | Neuroblast differentiation-associated | VDIDTPDIDIHGPEGK |
| | (T4100); | HUMAN | protein AHNAK (Desmoyokin) | [SEQ ID NO: 68] |
| Panel A | PRKAG2 | AAKG2_ | 5′-AMP-activated protein kinase subunit | IYASSSPPDTGQR |
| | (S196); | HUMAN | gamma-2 (AMPK gamma2) (AMPK subunit | [SEQ ID NO: 69] |
| | | | gamma-2) (H91620p) | |
| Panel C | CORO7 | CORO7_ | Coronin-7 (Crn7) (70 kDa WD repeat tumor | RESWISDIR |
| | (S21); | HUMAN | rejection antigen homolog) | [SEQ ID NO: 70] |
| Panel A | EIF4EBP1 | 4EBP1_ | Eukaryotic translation initiation factor | NSPVTKTPPRDLPTIP |
| | (S85); | HUMAN | 4E-binding protein 1 (4E-BP1) (elF4E- | GVTSPSSDEPPMEASQ |
| | EIF4EBP1 | | binding protein 1) (Phosphorylated heat- | SHLR |
| | (S86); | | and acid-stable protein regulated by | [SEQ ID NO: 71] |
| | | | insulin 1) (PHAS-1) | |
| Panel A | EIF4EBP2 | 4EBP2_ | Eukaryotic translation initiation factor | TVAISDAAQLPHDYCT |
| | (Y34); | HUMAN | 4E-binding protein 2 (4E-BP2) (elF4E- | TPGGTLFSTTPGGTR |
| | EIF4EBP2 | | binding protein 2) | [SEQ ID NO: 72] |
| | (T46); | | | |
| Panel A | AKT1S1 | AKTS1_ | Proline-rich AKT1 substrate 1 (40 kDa | SLPVSVPVWGFK |
| | (S183); | HUMAN | proline-rich AKT substrate) | [SEQ ID NO: 73] |
| Panel C | ATM | ATM_ | Serine-protein kinase ATM (EC 2.7.11.1) | NLSDIDQSFNK |
| | (S2996); | HUMAN | (Ataxia telangiectasia mutated) (A-T | [SEQ ID NO: 74] |
| | | | mutated) | |
| Panel D | BRAF | BRAF_ | Serine/threonine-protein kinase B-raf | DRSSSAPNVHINTIEP |
| | (S363); | HUMAN | (EC 2.7.11.1) (Proto-oncogene B-Raf) | VNIDDLIR |
| | | | (p94) (v-Raf murine sarcoma viral | [SEQ ID NO: 75] |
| | | | oncogene homolog B1) | |
| Panel A | CASP9 | CASP9_ | Caspase-9 (CASP-9) (EC 3.4.22.62) | DHGFEVASTSPEDESP |
| | (S310); | HUMAN | (Apoptotic protease Mch-6) (Apoptotic | GSNPEPDATPFQEGLR |
| | | | protease-activating factor 3) (APAF-3) | [SEQ ID NO: 76] |
| | | | (ICE-like apoptotic protease 6) (ICE- | |
| | | | LAP6) [Cleaved into: Caspase-9 subunit | |
| | | | p35; Caspase-9 subunit p10] | |
| Panel D | CDK13 | CDK13_ | Cyclin-dependent kinase 13 (EC 2.7.11.22) | KDLSLGLDDSRTNTPQ |
| | (T1058); | HUMAN | (EC 2.7.11.23) (CDC2-related protein | GVLPSSQLK |
| | CDK13 | | kinase 5) (Cell division cycle 2-like | [SEQ ID NO: 77] |
| | (S1065); | | protein kinase 5) (Cell division protein | |
| | | | kinase 13) (hCDK13) (Cholinesterase- | |
| | | | related cell division controller) | |
| Panel A | EPN1 | EPN1_ | Epsin-1 (EH domain-binding mitotic | GSLAEAVGSPPPAATP |
| | (S454); | HUMAN | phosphoprotein) (EPS-15-interacting | TPTPPTRK |
| | | | protein 1) | [SEQ ID NO: 78] |
| Panel D | GSK3A | GSK3A_ | Glycogen synthase kinase-3 alpha (GSK-3 | ARTSSFAEPGGGGGGG |
| | (S21); | HUMAN | alpha) (EC 2.7.11.26) (Serine/threonine- | GGGGGGSASGPGGTGG |
| | | | protein kinase GSK3A) (EC 2.7.11.1) | K |
| | | | | [SEQ ID NO: 79] |
| Panel A | GYS1 | GYS1_ | Glycogen [starch] synthase, muscle | NSVDTATSSSLSTPSE |
| | (S730); | HUMAN | (EC 2.4.1.11) | PLSPTSSLGEERN |
| | GYS1 | | | [SEQ ID NO: 80] |
| | (S731); | | | |
| Panel D | H2AFY | H2AY_ | Core histone macro-H2A.1 (Histone | SIAFPSIGSGR |
| | (S316); | HUMAN | macroH2A1) (mH2A1) (Histone H2A.y) | [SEQ ID NO: 81] |
| | | | (H2A/y) (Medulloblastoma antigen | |
| | | | MU-MB-50.205) | |
| Panel D | JAK3 | JAK3_ | Tyrosine-protein kinase JAK3 (EC | SCSLLSTEAGALHVLL |
| | (S20); | HUMAN | 2.7.10.2) (Janus kinase 3) (JAK-3) | PAR |
| | JAK3 | | (Leukocyte janus kinase) (L-JAK) | [SEQ ID NO: 82] |
| | (T21); | | | |
| Panel D | KDM2B | KDM2B_ | Lysine-specific demethylase 2B (EC | APKPPTDGSTSPTSTP |
| | (S1029); | HUMAN | 1.14.11.27) (CXXC-type zinc finger | SEDQEALGK |
| | | | protein 2) (F-box and leucine-rich | [SEQ ID NO: 83] |
| | | | repeat protein 10) (F-box protein | |
| | | | FBL10) (F-box/LRR-repeat protein | |
| | | | 10) (JmjC domain-containing histone | |
| | | | demethylation protein 1B) (Jumonji | |
| | | | domain-containing EMSY-interactor | |
| | | | methyltransferase motif protein) | |
| | | | (Protein JEMMA) (Protein-containing | |
| | | | CXXC domain 2) ([Histone-H3]-lysine-36 | |
| | | | demethylase 1B) | |
| Panel B | MLL2 | KMT2D_ | Histone-lysine N-methyltransferase 2D | QQQQQQQQQQQQQQHS |
| | (S3622); | HUMAN | (Lysine N-methyltransferase 2D) (EC | AVLALSPSQSPR |
| | | | 2.1.1.364) (ALL1-related protein) | [SEQ ID NO: 84] |
| | | | (Myeloid/lymphoid or mixed-lineage | |
| | | | leukemia protein 2) | |
| Panel D | PRKCD | KPCD_ | Protein kinase C delta type (EC | ASTFCGTPDYIAPEIL |
| | (S506) | HUMAN | 2.7.11.13) (Tyrosine-protein kinase | QGLK |
| | and/or | | PRKCD)(EC 2.7.10.2) (nPKC-delta) [Cleaved | [SEQ ID NO: 85] |
| | PRKCD | | into: Protein kinase C delta type regu- | |
| | (T507); | | latory subunit; Protein kinase C delta | |
| | | | type catalytic subunit (Sphingosine- | |
| | | | dependent protein kinase-1) (SDK1)] | |
| Panel D | CAMK1D | KCC1D_ | Calcium/calmodulin-dependent protein | GDVMSTACGTPGYVAP |
| | (S179); | HUMAN | kinase type 1D (EC 2.7.11.17) (CaM | EVLAQKPYSK |
| | | | kinase I delta) (CaM kinase ID) (CaM-KI | [SEQ ID NO: 86] |
| | | | delta) (CaMKI delta) (CaMKID) (CaMKI- | |
| | | | like protein kinase) (CKLiK) | |
| Panel D | PRKCD | KPCD_ | Protein kinase C delta type (EC | SDSASSEPVGIYQGFE |
| | (S302); | HUMAN | 2.7.11.13) (Tyrosine-protein kinase | K |
| | | | PRKCD) (EC 2.7.10.2) (nPKC-delta) | [SEQ ID NO: 87] |
| | | | [Cleaved into: Protein kinase C delta | |
| | | | type regulatory subunit; Protein | |
| | | | kinase C delta type catalytic subunit | |
| | | | (Sphingosine-dependent protein kinase-1) | |
| | | | (SDK1)] | |
| Panel B | MARCKS | MARCS_ | Myristoylated alanine-rich C-kinase | GEPAAAAAPEAGASPV |
| | (S128); | HUMAN | substrate (MARCKS) (Protein kinase C | EKEAPAEGEAAEPGSP |
| | | | substrate, 80 kDa protein, light chain) | TAAEGEAASAASSTSS |
| | | | (80K-L protein) (PKCSL) | PK |
| | | | | [SEQ ID NO: 88] |
| Panel D | MINK1 | MINK1_ | Misshapen-like kinase 1 (EC 2.7.11.1) | VGVSSKPDSSPVLSPG |
| | (S778); | HUMAN | (GCK family kinase MiNK) (MAPK/ERK | NK |
| | | | kinase kinase kinase 6) (MEK kinase | [SEQ ID NO: 89] |
| | | | kinase 6) (MEKKK 6) (Misshapen/NIK- | |
| | | | related kinase) (Mitogen-activated | |
| | | | protein kinase kinase kinase kinase 6) | |
| Panel C | MYC | MYC_ | Myc proto-oncogene protein (Class E | KFELLPTPPLSPSRR |
| | (T58); | HUMAN | basic helix-loop-helix protein 39) | [SEQ ID NO: 90] |
| | MYC | | (bHLHe39) (Proto-oncogene c-Myc) | |
| | (S64);/ | | (Transcription factor p64) | |
| | MYCN | | | |
| | (T58); | | | |
| | MYCN | | | |
| | (S64); | | | |
| Panel B | PSD3 | PSD3_ | PH and SEC7 domain-containing protein 3 | SHSSPSLNPDTSPITA |
| | (S1014); | HUMAN | (Epididymis tissue protein Li 20mP) | K |
| | | | (Exchange factor for ADP-ribosylation | [SEQ ID NO: 91] |
| | | | factor guanine nucleotide factor 6 D) | |
| | | | (Exchange factor for ARF6 D) (Hepatocel- | |
| | | | lular carcinoma-associated antigen 67) | |
| | | | (Pleckstrin homology and SEC7 domain- | |
| | | | containing protein 3) | |
| Panel A | STAT1 | STAT1_ | Signal transducer and activator of | LQTTDNLLPMSPEEFD |
| | (S727); | HUMAN | transcription 1-alpha/beta (Trans- | EVSR |
| | | | cription factor ISGF-3 components p91/p84) | [SEQ ID NO: 92] |
| Panel C | VIM | VIME_ | Vimentin | QVQSLTCEVDALKGTN |
| | (S339); | HUMAN | | ESLER |
| | | | | [SEQ ID NO: 93] |
| Panel D | ZNF608 | ZN608_ | Zinc finger protein 608 | APASPGAGNPPGTPK |
| | (S627); | HUMAN | | [SEQ ID NO: 94] |

Clinical utility may be improved by using comparing the levels of two of biomarkers. The biomarkers are typically of the same type, in other words the method may further comprise comparing the level of a first protein with the level of a second protein and/or comparing the level of phosphorylation at a first phosphorylation site with the level of phosphorylation at a second phosphorylation site. The first protein or phosphorylation site is typically a protein with a high level, or a phosphorylation site with a high level, in midostaurin responders. The second protein or phosphorylation site is typically a protein with a low level or not a high level, or a phosphorylation site with a low level or not a high level in midostaurin responders. The comparison may be expressed as a ratio or as a single number arrived at by dividing (or subtracting) the level of the first biomarker by the level of the second biomarker. Accordingly, when expressed as a single number, the comparison may produce a positive value and/or a value greater than one in predicted midostaurin responders. The number derived from the comparison may be described herein as the “response index”.

The method may therefore comprise a further step comprising comparing the level of a first protein of the one or more proteins with the level of a second protein of the one or more proteins and/or comparing the level of phosphorylation at a first phosphorylation site of the one or more phosphorylation sites with the level of phosphorylation at a second phosphorylation site of the one or more phosphorylation sites.

The comparing may be by dividing the level of the first protein by the level of the second protein and/or by dividing the level of phosphorylation at the first phosphorylation site from the level of phosphorylation at the second phosphorylation site.

The comparing may be by subtracting from the level of the first protein the level of the second protein and/or by subtracting from the level of phosphorylation at the first phosphorylation site by the level of phosphorylation at the second phosphorylation site.

Hence, if the level of a first protein of the one or more proteins and/or the level of phosphorylation at a first phosphorylation site of the one or more phosphorylation sites is high or low relative to the level a second protein of the one or more proteins and/or the level of phosphorylation at a second phosphorylation site of the one or more phosphorylation sites, predicting that the cancer, such as acute myeloid leukaemia, in the patient may be effectively treated with midostaurin.

“High relative to” herein may mean division of the level of the first biomarker by the level of the second biomarker provides a value greater than one—i.e. greater than unity. The method may comprise predicting that a cancer, such as acute myeloid leukaemia, in the patient may be effectively treated with midostaurin—either alone or in combination with chemotherapy— when the comparison between the first and second biomarkers results in a response index greater than one. Alternatively, a threshold response index may be applied. For example, the method may comprise predicting that the acute myeloid leukaemia in the patient may be effectively treated with midostaurin when the comparison between the first and second biomarkers results in a response index of at least 1.1, at least 1.2, at least 1.3, at least 1.4, at least 1.5, at least 1.6, at least 1.7, at least 1.8, at least 1.9, at least 2, at least 3, at least 4, at least 5 or at least 10.

“High relative to” herein may alternatively mean subtracting from the level of the first biomarker the level of the second biomarker provides a value greater than zero. The method may comprise predicting that the acute myeloid leukaemia in the patient may be effectively treated with midostaurin when the comparison between the first and second biomarkers results in a response index greater than zero. Alternatively, a threshold response index may be applied. For example, the method may comprise predicting that the acute myeloid leukaemia in the patient may be effectively treated with midostaurin when the comparison between the first and second biomarkers results in a response index of at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 3, at least 4, at least 5 or at least 10.

“Low relative to” herein may mean division of the level of the first biomarker by the level of the second biomarker provides a value less than one. The method may comprise predicting that the acute myeloid leukaemia in the patient may not be effectively treated with midostaurin when the comparison between the first and second biomarkers results in a response index less than one. Alternatively, a threshold response index may be applied. For example, the method may comprise predicting that the acute myeloid leukaemia in the patient may not be effectively treated with midostaurin when the comparison between the first and second biomarkers results in a response index of less than 0.9, less than 0.8, less than 0.7, less than 0.6, less than 0.5, less than 0.4, less than 0.3, less than 0.2 or less than 0.1.

“Low relative to” herein may mean subtraction of the level of the first biomarker by the level of the second biomarker provides a value less than zero. The method may comprise predicting that the acute myeloid leukaemia in the patient may not be effectively treated with midostaurin when the comparison between the first and second biomarkers results in a response index less than zero. Alternatively, a threshold response index may be applied. For example, the method may comprise predicting that the acute myeloid leukaemia in the patient may not be effectively treated with midostaurin when the comparison between the first and second biomarkers results in a response index of less than −10, less than −5, less than −2, less than −1, less than −0.9, less than −0.8, less than −0.7, less than −0.6, less than −0.5, less than −0.4, less than −0.3, less than −0.2 or less than −0.1.

The first protein of the one or more proteins and the second protein of the one or more proteins are different. The first phosphorylation site of the one or more phosphorylation sites and the second phosphorylation site of the one or more phosphorylation sites are different.

In one embodiment, if the level of a first protein of the one or more proteins and/or the level of phosphorylation at a first phosphorylation site of the one or more phosphorylation sites is high relative to the level a second protein of the one or more proteins and/or the level of phosphorylation at a second phosphorylation site of the one or more phosphorylation sites, the method provides for predicting that the cancer, such as acute myeloid leukaemia, in the patient may be effectively treated with midostaurin, or midostaurin in combination with chemotherapy.

In a further embodiment, if the level of a first protein of the one or more proteins is high relative to the level a second protein of the one or more proteins, the method provides for predicting that the cancer, such as acute myeloid leukaemia, in the patient may be effectively treated with midostaurin, or midostaurin in combination with chemotherapy.

In yet a further embodiment, if the level of phosphorylation at a first phosphorylation site of the one or more phosphorylation sites is high relative to the level of phosphorylation at a second phosphorylation site of the one or more phosphorylation sites, the method provides for predicting that the cancer, such as acute myeloid leukaemia, in the patient may be effectively treated with midostaurin, or midostaurin in combination with chemotherapy.

The first protein of the one or more proteins may be selected from any of the proteins set below:
Heterogeneous nuclear ribonucleoprotein M
Protein PML (E3 SUMO-protein ligase PML) (EC 2.3.2.-) (Promyelocytic leukemia protein) (RING finger protein 71) (RING-type E3 SUMO transferase PML) (Tripartite motif-containing protein 19) (TRIM19)
Neuroblast differentiation-associated protein AHNAK
Myoferlin
Dedicator of cytokinesis protein 10 (Zizimin-3)
Eukaryotic translation initiation factor 3 subunit D
Glutamine-fructose-6-phosphate aminotransferase
Choline-phosphate cytidylyltransferase A
V-type proton ATPase subunit B, brain isoform
Eukaryotic translation initiation factor 2 subunit 3
V-type proton ATPase catalytic subunit A

Alternatively, the first protein of the one or more proteins may be selected from any of the proteins set below:
Integrin alpha-5
Glutathione S-transferase theta-2
Arginine-tRNA ligase, cytoplasmic
Filensin
Protein un-13 homolog D
V-type proton ATPase subunit B, brain isoform
Alpha-1,6-mannosyl-glycoprotein 2-beta-N-acetylglucosaminyttransferase
Arfaptin-1
C—C motif chemokine 3
UDP-N-acetylhexosamine pyrophosphorylase-like protein 1
Eukaryotic translation initiation factor 2 subunit 3
Glutathione synthetase
V-type proton ATPase catalytic subunit A
Sodium/potassium-transporting ATPase subunit alpha-1
Serine/threonine-protein kinase MRCK beta
Protein YIPF4
DnaJ homolog subfamily C member 2
Immunoglobulin lambda-1 light chain
(Lyso)-N-acylphosphatidylethanolamine lipase
Ribosome biogenesis protein BMS1 homolog
Rho GTPase-activating protein 26; and
Patatin-like phospholipase domain-containing protein 6.

The second protein of the one or more proteins may be selected from the group consisting of:
Probable rRNA-processing protein EBP2
RNA exonuclease 4
SAP30-binding protein
2-aminomuconic semialdehyde dehydrogenase
Extracellular matrix protein 1
CCA tRNA nucleotidyltransferase 1, mitochondrial
BTB/POZ domain-containing protein 16
Kalirin
Ribosomal oxygenase 1
Oxysterol-binding protein-related protein 9
ATP-dependent DNA helicase Q1
Syntaxin-12
Protein MEMO1
Caspase recruitment domain-containing protein 19
Calcium/calmodulin-dependent protein kinase type 1D
Adhesion G protein-coupled receptor A1; and
AMP deaminase 3.

The first protein of the one or more proteins may be selected from the group consisting of:
Arginine-tRNA ligase, cytoplasmic;
Integrin alpha-5;
V-type proton ATPase subunit B, brain isoform; and
Protein unc-13 homolog D.

The second protein of the one or more proteins may be selected from the group consisting of:
Probable rRNA-processing protein EBP2;
SAP30-binding protein;
CCA tRNA nucleotidyltransferase 1, mitochondrial; and
ATP-dependent DNA helicase Q1.

In certain embodiments, the method may comprise comparing the level of any one of Arginine-tRNA ligase, cytoplasmic; Integrin alpha-5; V-type proton ATPase subunit B, brain isoform; and Protein unc-13 homolog D with the level of any one of Probable rRNA-processing protein EBP2; SAP30-binding protein; CCA tRNA nucleotidyftransferase 1, mitochondrial; and ATP-dependent DNA helicase Q1.

The comparing may comprise dividing the level of the first protein by the level of the second protein. Accordingly, the method may comprise dividing the level of any one of Arginine-tRNA ligase, cytoplasmic; Integrin alpha-5; V-type proton ATPase subunit B, brain isoform; and Protein unc-13 homolog D by the level of any one of Probable rRNA-processing protein EBP2; SAP30-binding protein; CCA tRNA nucleotidyltransferase 1, mitochondrial; and ATP-dependent DNA helicase Q1.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein;
Arginine-tRNA ligase, cytoplasmic with CCA tRNA nucleotidyltransferase 1, mitochondrial;
V-type proton ATPase subunit B, brain isoform with CCA tRNA nucleotidyltransferase 1, mitochondrial;
Integrin alpha-5 with CCA tRNA nucleotidyltransferase 1, mitochondrial;
Arginine-tRNA ligase, cytoplasmic with SAP30-binding protein;
V-type proton ATPase subunit B, brain isoform with Probable rRNA-processing protein EBP2;
V-type proton ATPase subunit B, brain isoform with SAP30-binding protein;
Protein unc-13 homolog D with ATP-dependent DNA helicase Q1;
Protein unc-13 homolog D with Probable rRNA-processing protein EBP2;
Protein unc-13 homolog D with SAP30-binding protein;
Integrin alpha-5 with ATP-dependent DNA helicase Q1;
Arginine-tRNA ligase, cytoplasmic with ATP-dependent DNA helicase Q1;
Protein unc-13 homolog D with CCA tRNA nucleotidyltransferase 1, mitochondrial; or
V-type proton ATPase subunit B, brain isoform with ATP-dependent DNA helicase Q1.

The method may comprise:
Dividing the level of Arginine-tRNA ligase, cytoplasmic by the level of Probable rRNA-processing protein EBP2;
Dividing the level of Integrin alpha-5 by the level of Probable rRNA-processing protein EBP2;
Dividing the level of Integrin alpha-5 by the level of SAP30-binding protein;
Dividing the level of Arginine-tRNA ligase, cytoplasmic the level of CCA tRNA nucleotidyltransferase 1, mitochondrial;
Dividing the level of V-type proton ATPase subunit B, brain isoform by the level of CCA tRNA nucleotidyltransferase 1, mitochondrial;
Dividing the level of Integrin alpha-5 by the level of CCA tRNA nucleotidyltransferase 1, mitochondrial;
Dividing the level of Arginine-tRNA ligase, cytoplasmic by the level of SAP30-binding protein;
Dividing the level of V-type proton ATPase subunit B, brain isoform by the level of Probable rRNA-processing protein EBP2;
Dividing the level of V-type proton ATPase subunit B, brain isoform by the level of SAP30-binding protein;
Dividing the level of Protein unc-13 homolog D by the level of ATP-dependent DNA helicase Q1;
Dividing the level of Protein unc-13 homolog D by the level of Probable rRNA-processing protein EBP2;
Dividing the level of Protein unc-13 homolog D by the level of SAP30-binding protein;
Dividing the level of Integrin alpha-5 by the level of ATP-dependent DNA helicase Q1;
Dividing the level of Arginine-tRNA ligase, cytoplasmic by the level of ATP-dependent
DNA helicase Q1;
Dividing the level of Protein unc-13 homolog D by the level of CCA tRNA nucleotidyltransferase 1, mitochondrial; or
Dividing the level of V-type proton ATPase subunit B, brain isoform by the level of ATP-dependent DNA helicase Q1.

As described herein, the comparing may be by subtracting, rather than by dividing. Any reference to the method comprising dividing the level of one biomarker by the level of another biomarker may alternatively be stated as subtracting from the level of one biomarker the level of another biomarker.

For example, the method may comprise:
Subtracting from the level of Arginine-tRNA ligase, cytoplasmic by the level of Probable rRNA-processing protein EBP2;
Subtracting from the level of Integrin alpha-5 by the level of Probable rRNA-processing protein EBP2;
Subtracting from the level of Integrin alpha-5 by the level of SAP30-binding protein;
Subtracting from the level of Arginine-tRNA ligase, cytoplasmic the level of CCA tRNA nucleotidyltransferase 1, mitochondrial;
Subtracting from the level of V-type proton ATPase subunit B, brain isoform by the level of CCA tRNA nucleotidyltransferase 1, mitochondrial;
Subtracting from the level of Integrin alpha-5 by the level of CCA tRNA nucleotidyltransferase 1, mitochondrial;
Subtracting from the level of Arginine-tRNA ligase, cytoplasmic by the level of SAP30-binding protein;
Subtracting from the level of V-type proton ATPase subunit B, brain isoform by the level of Probable rRNA-processing protein EBP2;
Subtracting from the level of V-type proton ATPase subunit B, brain isoform by the level of SAP30-binding protein;
Subtracting from the level of Protein unc-13 homolog D by the level of ATP-dependent DNA helicase Q1;
Subtracting from the level of Protein unc-13 homolog D by the level of Probable rRNA-processing protein EBP2;
Subtracting from the level of Protein unc-13 homolog D by the level of SAP30-binding protein;
Subtracting from the level of Integrin alpha-5 by the level of ATP-dependent DNA helicase Q1;
Subtracting from the level of Arginine-tRNA ligase, cytoplasmic by the level of ATP-dependent DNA helicase Q1;
Subtracting from the level of Protein unc-13 homolog D by the level of CCA tRNA nucleotidyltransferase 1, mitochondrial; or
Subtracting from the level of V-type proton ATPase subunit B, brain isoform by the level of ATP-dependent DNA helicase Q1.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein;
Arginine-tRNA ligase, cytoplasmic with CCA tRNA nucleotidyltransferase 1, mitochondrial;
V-type proton ATPase subunit B, brain isoform with CCA tRNA nucleotidyltransferase 1, mitochondrial;
Integrin alpha-5 with CCA tRNA nucleotidyltransferase 1, mitochondrial;
Arginine-tRNA ligase, cytoplasmic with SAP30-binding protein;
V-type proton ATPase subunit B, brain isoform with Probable rRNA-processing protein EBP2;
V-type proton ATPase subunit B, brain isoform with SAP30-binding protein;
Protein unc-13 homolog D with ATP-dependent DNA helicase Q1;
Protein unc-13 homolog D with Probable rRNA-processing protein EBP2:
Protein unc-13 homolog D with SAP30-binding protein; or
Integrin alpha-5 with ATP-dependent DNA helicase Q1.

The method may comprise:
Dividing the level of Arginine-tRNA ligase, cytoplasmic the level of Probable rRNA-processing protein EBP2;
Dividing the level of Integrin alpha-5 the level of Probable rRNA-processing protein EBP2;
Dividing the level of Integrin alpha-5 by the level of SAP30-binding protein;
Dividing the level of Arginine-tRNA ligase, cytoplasmic by the level of CCA tRNA nucleotidyltransferase 1, mitochondrial;
Dividing the level of V-type proton ATPase subunit B, brain isoform by the level of CCA tRNA nucleotidyltransferase 1, mitochondrial;
Dividing the level of Integrin alpha-5 by the level of CCA tRNA nucleotidyltransferase 1, mitochondrial;
Dividing the level of Arginine-tRNA ligase, cytoplasmic by the level of SAP30-binding protein;
Dividing the level of V-type proton ATPase subunit B, brain isoform by the level of Probable rRNA-processing protein EBP2;
Dividing the level of V-type proton ATPase subunit B, brain isoform by the level of SAP30-binding protein;
Dividing the level of Protein unc-13 homolog D by the level of ATP-dependent DNA helicase Q1;
Dividing the level of Protein unc-13 homolog D by the level of Probable rRNA-processing protein EBP2;
Dividing the level of Protein unc-13 homolog D by the level of SAP30-binding protein; or
Dividing the level of Integrin alpha-5 by the level of ATP-dependent DNA helicase Q1.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein;
Arginine-tRNA ligase, cytoplasmic with CCA tRNA nucleotidyltransferase 1, mitochondrial;
V-type proton ATPase subunit B, brain isoform with CCA tRNA nucleotidyltransferase 1, mitochondrial;
Integrin alpha-5 with CCA tRNA nucleotidyltransferase 1, mitochondrial;
Arginine-tRNA ligase, cytoplasmic with SAP30-binding protein; or
V-type proton ATPase subunit B, brain isoform with Probable rRNA-processing protein EBP2.

The method may comprise:
Dividing the level of Arginine-tRNA ligase, cytoplasmic by the level of Probable rRNA-processing protein EBP2;
Dividing the level of Integrin alpha-5 by the level of Probable rRNA-processing protein EBP2;
Dividing the level of Integrin alpha-5 by the level of SAP30-binding protein;
Dividing the level of Arginine-tRNA ligase, cytoplasmic by the level of CCA tRNA nucleotidyltransferase 1, mitochondrial;
Dividing the level of V-type proton ATPase subunit B, brain isoform by the level of CCA tRNA nucleotidyltransferase 1, mitochondrial;
Dividing the level of Integrin alpha-5 by the level of CCA tRNA nucleotidyltransferase 1, mitochondrial;
Dividing the level of Arginine-tRNA ligase, cytoplasmic by the level of SAP30-binding protein; or
Dividing the level of V-type proton ATPase subunit B, brain isoform by the level of Probable rRNA-processing protein EBP2.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein;
Arginine-tRNA ligase, cytoplasmic with CCA tRNA nucleotidyltransferase 1, mitochondrial;
V-type proton ATPase subunit B, brain isoform with CCA tRNA nucleotidyltransferase 1, mitochondrial;
Integrin alpha-5 with CCA tRNA nucleotidyltransferase 1, mitochondrial;
Arginine-tRNA ligase, cytoplasmic with SAP30-binding protein; or
V-type proton ATPase subunit B, brain isoform with Probable rRNA-processing protein EBP2.

The method may comprise:
Dividing the level of Arginine-tRNA ligase, cytoplasmic by the level of Probable rRNA-processing protein EBP2;
Dividing the level of Integrin alpha-5 by the level of Probable rRNA-processing protein EBP2;
Dividing the level of Integrin alpha-5 by the level of SAP30-binding protein;
Dividing the level of Arginine-tRNA ligase, cytoplasmic by the level of CCA tRNA nucleotidyftransferase 1, mitochondrial;
Dividing the level of V-type proton ATPase subunit B, brain isoform by the level of CCA tRNA nucleotidyltransferase 1, mitochondrial:
Dividing the level of Integrin alpha-5 by the level of CCA tRNA nucleotidyRtransferase 1, mitochondrial;
Dividing the level of Arginine-tRNA ligase, cytoplasmic by the level of SAP30-binding protein; or
Dividing the level of V-type proton ATPase subunit B, brain isoform by the level of Probable rRNA-processing protein EBP2.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein;
Arginine-tRNA ligase, cytoplasmic with CCA tRNA nucleotidyltransferase 1, mitochondrial;
V-type proton ATPase subunit B, brain isoform with CCA tRNA nucleotidyltransferase 1, mitochondrial; or
Integrin alpha-5 with CCA tRNA nucleotidyltransferase 1, mitochondrial.

The method may comprise:
Dividing the level of Arginine-tRNA ligase, cytoplasmic by the level of Probable rRNA-processing protein EBP2;
Dividing the level of Integrin alpha-5 by the level of Probable rRNA-processing protein EBP2;
Dividing the level of Integrin alpha-5 by the level of SAP30-binding protein;
Dividing the level of Arginine-tRNA ligase, cytoplasmic the level of CCA tRNA nucleotidyltransferase 1, mitochondrial;
Dividing the level of V-type proton ATPase subunit B, brain isoform by the level of CCA tRNA nucleotidyltransferase 1, mitochondrial; or
Dividing the level of Integrin alpha-5 by the level of CCA tRNA nucleotidyltransferase 1, mitochondrial.

The method may comprise comparing the levels of
Arginine-tRNA ligase, cytoplasmic with Probable rRNA-processing protein EBP2;
Integrin alpha-5 with Probable rRNA-processing protein EBP2;
Integrin alpha-5 with SAP30-binding protein; or
Arginine-tRNA ligase, cytoplasmic with CCA tRNA nucleotidyltransferase 1, mitochondrial.

The method may comprise:
Dividing the level of Arginine-tRNA ligase, cytoplasmic by the level of Probable rRNA-processing protein EBP2;
Dividing the level of Integrin alpha-5 by the level of Probable rRNA-processing protein EBP2;
Dividing the level of Integrin alpha-5 by the level of SAP30-binding protein; or
Dividing the level of Arginine-tRNA ligase, cytoplasmic by the level of CCA tRNA nucleotidyltransferase 1, mitochondrial.

The method may comprise:
Comparing the level of Arginine-tRNA ligase, cytoplasmic with the level of Probable rRNA-processing protein EBP2.

The method may comprise:
Comparing the level of Integrin alpha-5 with the level of Probable rRNA-processing protein EBP2.

The method may comprise:
Comparing the level of Integrin alpha-5 with the level of SAP30-binding protein.

The method may comprise:
Comparing the level of Arginine-tRNA ligase, cytoplasmic with the level of CCA tRNA nucleotidyltransferase 1, mitochondrial.

Wherein the sample is a peripheral blood sample, the method may comprise any one of:
Comparing the level of Arginine-tRNA ligase, cytoplasmic with the level of Probable rRNA-processing protein EBP2;
Comparing the level of Integrin alpha-5 with the level of Probable rRNA-processing protein EBP2;
Comparing the level of Integrin alpha-5 with the level of SAP30-binding protein;
Comparing the level of Arginine-tRNA ligase, cytoplasmic with the level of CCA tRNA nucleotidyltransferase 1, mitochondrial;
Comparing the level of V-type proton ATPase subunit B, brain isoform with the level of CCA tRNA nucleotidyltransferase 1, mitochondrial;
Comparing the level of Integrin alpha-5 with the level of CCA tRNA nucleotidyttransferase 1, mitochondrial;
Comparing the level of Arginine-tRNA ligase, cytoplasmic with the level of SAP30-binding protein; or
Comparing the level of V-type proton ATPase subunit B, brain isoform with the level of ATP-dependent DNA helicase Q1 Wherein the sample is a bone marrow sample, the method may comprise any one of:
Comparing the level of Arginine-tRNA ligase, cytoplasmic with the level of Probable rRNA-processing protein EBP2;
Comparing the level of Integrin alpha-5 with the level of Probable rRNA-processing protein EBP2;
Comparing the level of Integrin alpha-5 with the level of SAP30-binding protein;
Comparing the level of Arginine-tRNA ligase, cytoplasmic with the level of CCA tRNA nucleotidyltransferase 1, mitochondrial;
Comparing the level of V-type proton ATPase subunit B, brain isoform with the level of CCA tRNA nucleotidyltransferase 1, mitochondrial:
Comparing the level of Integrin alpha-5 with the level of CCA tRNA nucleotidyltransferase 1, mitochondrial;
Comparing the level of V-type proton ATPase subunit B, brain isoform with the level of Probable rRNA-processing protein EBP2;
Comparing the level of Protein unc-13 homolog D with the level of ATP-dependent DNA helicase Q1;
Comparing the level of Protein unc-13 homolog D with the level of Probable rRNA-processing protein EBP2;
Comparing the level of Protein unc-13 homolog D with the level of SAP30-binding protein;
Comparing the level of Integrin alpha-5 with the level of ATP-dependent DNA helicase Q1; or
Comparing the level of Protein unc-13 homolog D with the level of CCA tRNA nucleotidyltransferase 1, mitochondrial.

The first phosphorylation site of the one or more phosphorylation sites may be selected from the group consisting of any one of the sites set out in: Panels A to D; and/or Table 1; and/or Table 6.

Alternatively, in an embodiment, the first phosphorylation site of the one or more phosphorylation sites may be selected from any one of the phosphorylation sites set out below:
T719 of Signal transducer and activator of transcription 1-alpha/beta
T729 and/or S730 of Glycogen[starch] synthase, muscle;
T77 of Eukaryotic translation initiation factor 4E-binding protein 1;
T46 of Eukaryotic translation initiation factor 4E-binding protein 2;
S302 of Protein kinase C delta type;
S183 of Proline-rich AKT1 substrate 1;
T19 of Glycogen synthase kinase-3 alpha;
Y313 of Protein kinase C delta type
S363 of Serine/threonine-protein kinase B-raf;
S2996 of Serine-protein kinase ATM;
T327 of Vimentin;
S447 of Epsin-1;
S310 of Caspase-9; and
T120 of Myristoylated alanine-rich C-kinase substrate.

The first phosphorylation site of the one or more phosphorylation sites may be selected from the group consisting of:
T719 of Signal transducer and activator of transcription 1-alpha/beta
T729 and/or S730 of Glycogen[starch] synthase, muscle;
T77 of Eukaryotic translation initiation factor 4E-binding protein 1;
T46 of Eukaryotic translation initiation factor 4E-binding protein 2;
S302 of Protein kinase C delta type;
S183 of Proline-rich AKT1 substrate 1;
T19 of Glycogen synthase kinase-3 alpha;
S363 of Serine/threonine-protein kinase B-raf;
S2996 of Serine-protein kinase ATM;
T327 of Vimentin;
S447 of Epsin-1;
S310 of Caspase-9; and
T120 of Myristoylated alanine-rich C-kinase substrate.

The second phosphorylation site of the one or more phosphorylation sites may be selected from the group consisting of:
S627 of Zinc finger protein 608;
T58 of Myc proto-oncogene protein and/or T58 of N-myc proto-oncogene protein;
S316 of Core histone macro-H2A. 1;
S1029 of Lysine-specific demethylase 2B;
S3620 of Histone-lysine N-methyltransferase 20;
S15 and/or S17 of Tyrosine-protein kinase JAK3;
S1009 of PH and SEC7 domain-containing protein 3;
S1054 of Cyclin-dependent kinase 13;
S244 of DEP domain-containing mTOR-interacting protein;
S772 of Misshapen-like kinase 1;
S179 of Calcium/calmodulin-dependent protein kinase type 1D
S780 of Signal transducer and activator of transcription 5A; and
S506 of Protein kinase C delta type.

The second phosphorylation site of the one or more phosphorylation sites may be selected from the group consisting of:
S627 of Zinc finger protein 608;
T58 of Myc proto-oncogene protein and/or T58 of N-myc proto-oncogene protein;
S316 of Core histone macro-H2A. 1;
S1029 of Lysine-specific demethylase 2B;
S3620 of Histone-lysine N-methyltransferase 2D;
S15 and/or S17 of Tyrosine-protein kinase JAK3;
S1009 of PH and SEC7 domain-containing protein 3;
S1054 of Cyclin-dependent kinase 13;
S244 of DEP domain-containing mTOR-interacting protein;
S772 of Misshapen-like kinase 1;
S179 of Calcium/calmodulin-dependent protein kinase type 1D; and
S506 of Protein kinase C delta type.

The first phosphorylation site of the one or more phosphorylation sites may be selected from the group consisting of:
S183 of Proline-rich AKT1 substrate 1
T19 of Glycogen synthase kinase-3 alpha
T46 of Eukaryotic translation initiation factor 4E-binding protein 2
T719 of Signal transducer and activator of transcription 1-alpha/beta
Y313 of Protein kinase C delta type
S302 of Protein kinase C delta type
T719 of Signal transducer and activator of transcription 1-alpha/beta; and
Sum PKC GSK3 STAT1 4EBP2 AKT1S1;

Wherein sum PKC GSK3 STAT1 4EBP2 AKT1S1 refers to the sum of the following phosphorylation sites:
Y313 of Protein kinase C delta type
S302 of Protein kinase C delta type
T19 of Glycogen synthase kinase-3 alpha
T719 of Signal transducer and activator of transcription 1-alpha/beta
T46 of Eukaryotic translation initiation factor 4E-binding protein 2; and
S183 of Proline-rich AKT1 substrate 1.

The first phosphorylation site of the one or more phosphorylation sites may be selected from the group consisting of:
S183 of Proline-rich AKT1 substrate 1
T19 of Glycogen synthase kinase-3 alpha
T46 of Eukaryotic translation initiation factor 4E-binding protein 2
T719 of Signal transducer and activator of transcription 1-alpha/beta
S302 of Protein kinase C delta type
T719 of Signal transducer and activator of transcription 1-alpha/beta; and
Sum PKC GSK3 STAT1 4EBP2 AKT1S1;

Wherein sum PKC GSK3 STAT1 4EBP2 AKT1S1 refers to the sum of the following phosphorylation sites:
Y313 of Protein kinase C delta type
S302 of Protein kinase C delta type
T19 of Glycogen synthase kinase-3 alpha
T719 of Signal transducer and activator of transcription 1-alpha/beta
T46 of Eukaryotic translation initiation factor 4E-binding protein 2; and
S183 of Proline-rich AKT1 substrate 1.

The second phosphorylation site of the one or more phosphorylation sites may be selected from the group consisting of:
S627 of Zinc finger protein 608
S1054 of Cyclin-dependent kinase 13
S244 of DEP domain-containing mTOR-interacting protein
S780 of Signal transducer and activator of transcription 5A
T58 of Myc proto-oncogene protein and/or T58 of N-myc proto-oncogene protein; and
Sum CDK13 ZNF608 0 to 1;

Wherein sum CDK13 ZNF608 0 to 1 refers to the sum of the following phosphorylation sites:
S1054 of Cyclin-dependent kinase 13 and
S627 of Zinc finger protein 608.

The second phosphorylation site of the one or more phosphorylation sites may be selected from the group consisting of:
S627 of Zinc finger protein 608
S1054 of Cyclin-dependent kinase 13
S244 of DEP domain-containing mTOR-interacting protein
T58 of Myc proto-oncogene protein and/or T58 of N-myc proto-oncogene protein; and
Sum CDK13 ZNF608 0 to 1;

Wherein sum CDK13 ZNF608 0 to 1 refers to the sum of the following phosphorylation sites:
S1054 of Cyclin-dependent kinase 13 and
S627 of Zinc finger protein 608.

The method may comprise comparing the level of phosphorylation at any one of S183 of Proline-rich AKT1 substrate 1, T19 of Glycogen synthase kinase-3 alpha, T46 of Eukaryotic translation initiation factor 4E-binding protein 2, T719 of Signal transducer and activator of transcription 1-alpha/beta, Y313 of Protein kinase C delta type, S302 of Protein kinase C delta type, T719 of Signal transducer and activator of transcription 1-alpha/beta; and Sum PKC GSK3 STAT1 4EBP2 AKT1S1 to the level of phosphorylation at any one of S627 of Zinc finger protein 608, S1054 of Cyclin-dependent kinase 13, S244 of DEP domain-containing mTOR-interacting protein, S780 of Signal transducer and activator of transcription 5A, T58 of Myc proto-oncogene protein and/or T58 of N-myc proto-oncogene protein; and Sum CDK13 ZNF608 0 to 1.

The comparing may comprise dividing the level of phosphorylation at the first phosphorylation site by the level of phosphorylation at the second phosphorylation site. Accordingly, the method may comprise dividing the level of phosphorylation at any one of S183 of Proline-rich AKT1 substrate 1, T19 of Glycogen synthase kinase-3 alpha, T46 of Eukaryotic translation initiation factor 4E-binding protein 2, T719 of Signal transducer and activator of transcription 1-alpha/beta, Y313 of Protein kinase C delta type, S302 of Protein kinase C delta type, T719 of Signal transducer and activator of transcription 1-alpha/beta; and Sum PKC GSK3 STAT1 4EBP2 AKT1S1 by the level of phosphorylation at any one of S627 of Zinc finger protein 608, S1054 of Cyclin-dependent kinase 13, S244 of DEP domain-containing mTOR-interacting protein, S780 of Signal transducer and activator of transcription 5A, T58 of Myc proto-oncogene protein and/or T58 of N-myc proto-oncogene protein; and Sum CDK13 ZNF608 0 to 1.

The method may comprise comparing the levels of phosphorylation of
S183 of Proline-rich AKT1 substrate 1 with S627 of Zinc finger protein 608;
T19 of Glycogen synthase kinase-3 alpha with S627 of Zinc finger protein 608;
T46 of Eukaryotic translation initiation factor 4E-binding protein 2 with S627 of Zinc finger protein 608;
T719 of Signal transducer and activator of transcription 1-alpha/beta with S627 of Zinc finger protein 608;
T719 of Signal transducer and activator of transcription 1-alpha/beta with S1054 of Cyclin-dependent kinase 13;
Y313 of Protein kinase C delta type with S244 of DEP domain-containing mTOR-interacting protein;
Y313 of Protein kinase C delta type with S627 of Zinc finger protein 608;
T719 of Signal transducer and activator of transcription 1-alpha/beta with S780 of Signal transducer and activator of transcription 5A;
S302 of Protein kinase C delta type with S244 of DEP domain-containing mTOR-interacting protein;
T46 of Eukaryotic translation initiation factor 4E-binding protein 2 with S1054 of Cyclin-dependent kinase 13;
S183 of Proline-rich AKT1 substrate 1 with S1054 of Cyclin-dependent kinase 13;
S302 of Protein kinase C delta type with S627 of Zinc finger protein 608;
T19 of Glycogen synthase kinase-3 alpha with S780 of Signal transducer and activator of transcription 5A;
S183 of Proline-rich AKT1 substrate 1 with S780 of Signal transducer and activator of transcription 5A;
Y313 of Protein kinase C delta type with S780 of Signal transducer and activator of transcription 5A;
T719 of Signal transducer and activator of transcription 1-alpha/beta; with T58 of Myc proto-oncogene protein and/or T58 of N-myc proto-oncogene protein;
S302 of Protein kinase C delta type with S780 of Signal transducer and activator of transcription 5A;
T46 of Eukaryotic translation initiation factor 4E-binding protein 2 with S244 of DEP domain-containing mTOR-interacting protein;
S302 of Protein kinase C delta type with S1054 of Cyclin-dependent kinase 13;
T19 of Glycogen synthase kinase-3 alpha with S244 of DEP domain-containing mTOR-interacting protein;
Y313 of Protein kinase C delta type with S1054 of Cyclin-dependent kinase 13;
T719 of Signal transducer and activator of transcription 1-alpha/beta with S244 of DEP domain-containing mTOR-interacting protein;
S302 of Protein kinase C delta type with T58 of Myc proto-oncogene protein and/or T58 of N-myc proto-oncogene protein;
Ratio Sum PKC GSK3 STAT1 4EBP2 AKT1S1 with CDK13 ZNF608 0 to 1
T46 of Eukaryotic translation initiation factor 4E-binding protein 2 with S780 of Signal transducer and activator of transcription 5A;
T46 of Eukaryotic translation initiation factor 4E-binding protein 2 with T58 of Myc proto-oncogene protein and/or T58 of N-myc proto-oncogene protein;
S183 of Proline-rich AKT1 substrate 1 with S244 of DEP domain-containing mTOR-interacting protein;
T19 of Glycogen synthase kinase-3 alpha with S1054 of Cyclin-dependent kinase 13;
S183 of Proline-rich AKT1 substrate 1 with T58 of Myc proto-oncogene protein and/or T58 of N-myc proto-oncogene protein;
T19 of Glycogen synthase kinase-3 alpha with T58 of Myc proto-oncogene protein and/or
T58 of N-myc proto-oncogene protein; or
Y313 of Protein kinase C delta type with T58 of Myc proto-oncogene protein and/or T58 of N-myc proto-oncogene protein;

wherein sum PKC GSK3 STAT1 4EBP2 AKT1S1 refers to the sum of the following phosphorylation sites:
Y313 of Protein kinase C delta type
S302 of Protein kinase C delta type
T19 of Glycogen synthase kinase-3 alpha
T719 of Signal transducer and activator of transcription 1-alpha/beta
T46 of Eukaryotic translation initiation factor 4E-binding protein 2; and
S183 of Proline-rich AKT1 substrate 1;

and wherein sum CDK13 ZNF608 0 to 1 refers to the sum of the following phosphorylation sites:
S1054 of Cyclin-dependent kinase 13 and
S627 of Zinc finger protein 608.

The method may comprise:
Dividing the level of phosphorylation at S183 of Proline-rich AKT1 substrate 1 by the level of phosphorylation at S627 of Zinc finger protein 608;
Dividing the level of phosphorylation at T19 of Glycogen synthase kinase-3 alpha by the level of phosphorylation at S627 of Zinc finger protein 608;
Dividing the level of phosphorylation at T46 of Eukaryotic translation initiation factor 4E-binding protein 2 by the level of phosphorylation at S627 of Zinc finger protein 608;
Dividing the level of phosphorylation at T719 of Signal transducer and activator of transcription 1-alpha/beta by the level of phosphorylation at S627 of Zinc finger protein 608;
Dividing the level of phosphorylation at T719 of Signal transducer and activator of transcription 1-alpha/beta by the level of phosphorylation at S1054 of Cyclin-dependent kinase 13;
Dividing the level of phosphorylation at Y313 of Protein kinase C delta type by the level of phosphorylation at S244 of DEP domain-containing mTOR-interacting protein;
Dividing the level of phosphorylation at Y313 of Protein kinase C delta type by the level of phosphorylation at S627 of Zinc finger protein 608;
Dividing the level of phosphorylation at T719 of Signal transducer and activator of transcription 1-alpha/beta by the level of phosphorylation at S780 of Signal transducer and activator of transcription 5A;
Dividing the level of phosphorylation at S302 of Protein kinase C delta type by the level of phosphorylation at S244 of DEP domain-containing mTOR-interacting protein;
Dividing the level of phosphorylation at T46 of Eukaryotic translation initiation factor 4E-binding protein 2 by the level of phosphorylation at S1054 of Cyclin-dependent kinase 13;
Dividing the level of phosphorylation at S183 of Proline-rich AKT1 substrate 1 by the level of phosphorylation at S1054 of Cyclin-dependent kinase 13;
Dividing the level of phosphorylation at S302 of Protein kinase C delta type by the level of phosphorylation at S627 of Zinc finger protein 608;
Dividing the level of phosphorylation at T19 of Glycogen synthase kinase-3 alpha by the level of phosphorylation at S780 of Signal transducer and activator of transcription 5A;
Dividing the level of phosphorylation at S183 of Proline-rich AKT1 substrate 1 by the level of phosphorylation at S780 of Signal transducer and activator of transcription 5A;
Dividing the level of phosphorylation at Y313 of Protein kinase C delta type by the level of phosphorylation at S780 of Signal transducer and activator of transcription 5A;
Dividing the level of phosphorylation at T719 of Signal transducer and activator of transcription 1-alpha/beta; by the level of phosphorylation at T58 of Myc proto-oncogene protein and/or T58 of N-myc proto-oncogene protein;
Dividing the level of phosphorylation at S302 of Protein kinase C delta type by the level of phosphorylation at S780 of Signal transducer and activator of transcription 5A;
Dividing the level of phosphorylation at T46 of Eukaryotic translation initiation factor 4E-binding protein 2 by the level of phosphorylation at S244 of DEP domain-containing mTOR-interacting protein;
Dividing the level of phosphorylation at S302 of Protein kinase C delta type by the level of phosphorylation at S1054 of Cyclin-dependent kinase 13;
Dividing the level of phosphorylation at T19 of Glycogen synthase kinase-3 alpha by the level of phosphorylation at S244 of DEP domain-containing mTOR-interacting protein;
Dividing the level of phosphorylation at Y313 of Protein kinase C delta type by the level of phosphorylation at S1054 of Cyclin-dependent kinase 13;
Dividing the level of phosphorylation at T719 of Signal transducer and activator of transcription 1-alpha/beta by the level of phosphorylation at S244 of DEP domain-containing mTOR-interacting protein;
Dividing the level of phosphorylation at S302 of Protein kinase C delta type by the level of phosphorylation at T58 of Myc proto-oncogene protein and/or T58 of N-myc proto-oncogene protein;
Dividing the level of phosphorylation at Ratio Sum PKC GSK3 STAT1 4EBP2 AKT1S1 by the level of phosphorylation at CDK13 ZNF608 0 to 1
Dividing the level of phosphorylation at T46 of Eukaryotic translation initiation factor 4E-binding protein 2 by the level of phosphorylation at S780 of Signal transducer and activator of transcription 5A;
Dividing the level of phosphorylation at T46 of Eukaryotic translation initiation factor 4E-binding protein 2 by the level of phosphorylation at T58 of Myc proto-oncogene protein and/or T58 of N-myc proto-oncogene protein;
Dividing the level of phosphorylation at S183 of Proline-rich AKT1 substrate 1 by the level of phosphorylation at S244 of DEP domain-containing mTOR-interacting protein;
Dividing the level of phosphorylation at T19 of Glycogen synthase kinase-3 alpha by the level of phosphorylation at S1054 of Cyclin-dependent kinase 13;
Dividing the level of phosphorylation at S183 of Proline-rich AKT1 substrate 1 by the level of phosphorylation at T58 of Myc proto-oncogene protein and/or T58 of N-myc proto-oncogene protein;
Dividing the level of phosphorylation at T19 of Glycogen synthase kinase-3 alpha by the level of phosphorylation at T58 of Myc proto-oncogene protein and/or T58 of N-myc proto-oncogene protein;
Dividing the level of phosphorylation at Y313 of Protein kinase C delta type by the level of phosphorylation at T58 of Myc proto-oncogene protein and/or T58 of N-myc proto-oncogene protein;

As described herein, the comparing may be by subtracting, rather than by dividing. Any reference to the method comprising dividing the level of one biomarker by the level of another biomarker may alternatively be stated as subtracting from the level of one biomarker the level of another biomarker.

For example, the method may comprise:
Subtracting from the level of phosphorylation at S183 of Proline-rich AKT1 substrate 1 the level of phosphorylation at S627 of Zinc finger protein 608;
Subtracting from the level of phosphorylation at T19 of Glycogen synthase kinase-3 alpha the level of phosphorylation at S627 of Zinc finger protein 608;
Subtracting from the level of phosphorylation at T46 of Eukaryotic translation initiation factor 4E-binding protein 2 the level of phosphorylation at S627 of Zinc finger protein 608;
Subtracting from the level of phosphorylation at T719 of Signal transducer and activator of transcription 1-alpha/beta the level of phosphorylation at S627 of Zinc finger protein 608;
Subtracting from the level of phosphorylation at T719 of Signal transducer and activator of transcription 1-alpha/beta the level of phosphorylation at S1054 of Cyclin-dependent kinase 13;
Subtracting from the level of phosphorylation at Y313 of Protein kinase C delta type the level of phosphorylation at S244 of DEP domain-containing mTOR-interacting protein;
Subtracting from the level of phosphorylation at Y313 of Protein kinase C delta type the level of phosphorylation at S627 of Zinc finger protein 608;
Subtracting from the level of phosphorylation at T719 of Signal transducer and activator of transcription 1-alpha/beta the level of phosphorylation at S780 of Signal transducer and activator of transcription 5A;
Subtracting from the level of phosphorylation at S302 of Protein kinase C delta type the level of phosphorylation at S244 of DEP domain-containing mTOR-interacting protein;
Subtracting from the level of phosphorylation at T46 of Eukaryotic translation initiation factor 4E-binding protein 2 the level of phosphorylation at S1054 of Cyclin-dependent kinase 13;
Subtracting from the level of phosphorylation at S183 of Proline-rich AKT1 substrate 1 the level of phosphorylation at S1054 of Cyclin-dependent kinase 13;
Subtracting from the level of phosphorylation at S302 of Protein kinase C delta type the level of phosphorylation at S627 of Zinc finger protein 608;
Subtracting from the level of phosphorylation at T19 of Glycogen synthase kinase-3 alpha the level of phosphorylation at S780 of Signal transducer and activator of transcription 5A;
Subtracting from the level of phosphorylation at S183 of Proline-rich AKT1 substrate 1 the level of phosphorylation at S780 of Signal transducer and activator of transcription 5A;
Subtracting from the level of phosphorylation at Y313 of Protein kinase C delta type the level of phosphorylation at S780 of Signal transducer and activator of transcription 5A;
Subtracting from the level of phosphorylation at T719 of Signal transducer and activator of transcription 1-alpha/beta; the level of phosphorylation at T58 of Myc proto-oncogene protein and/or T58 of N-myc proto-oncogene protein;
Subtracting from the level of phosphorylation at S302 of Protein kinase C delta type the level of phosphorylation at S780 of Signal transducer and activator of transcription 5A;
Subtracting from the level of phosphorylation at T46 of Eukaryotic translation initiation factor 4E-binding protein 2 the level of phosphorylation at S244 of DEP domain-containing mTOR-interacting protein;
Subtracting from the level of phosphorylation at S302 of Protein kinase C delta type the level of phosphorylation at S1054 of Cyclin-dependent kinase 13;
Subtracting from the level of phosphorylation at T19 of Glycogen synthase kinase-3 alpha the level of phosphorylation at S244 of DEP domain-containing mTOR-interacting protein;
Subtracting from the level of phosphorylation at Y313 of Protein kinase C delta type the level of phosphorylation at S1054 of Cyclin-dependent kinase 13;
Subtracting from the level of phosphorylation at T719 of Signal transducer and activator of transcription 1-alpha/beta the level of phosphorylation at S244 of DEP domain-containing mTOR-interacting protein;
Subtracting from the level of phosphorylation at S302 of Protein kinase C delta type the level of phosphorylation at T58 of Myc proto-oncogene protein and/or T58 of N-myc proto-oncogene protein;
Subtracting from the level of phosphorylation at Ratio Sum PKC GSK3 STAT1 4EBP2 AKT1S1 the level of phosphorylation at CDK13 ZNF608 0 to 1
Subtracting from the level of phosphorylation at T46 of Eukaryotic translation initiation factor 4E-binding protein 2 the level of phosphorylation at S780 of Signal transducer and activator of transcription 5A;
Subtracting from the level of phosphorylation at T46 of Eukaryotic translation initiation factor 4E-binding protein 2 the level of phosphorylation at T58 of Myc proto-oncogene protein and/or T58 of N-myc proto-oncogene protein;
Subtracting from the level of phosphorylation at S183 of Proline-rich AKT1 substrate 1 the level of phosphorylation at S244 of DEP domain-containing mTOR-interacting protein;
Subtracting from the level of phosphorylation at T19 of Glycogen synthase kinase-3 alpha the level of phosphorylation at S1054 of Cyclin-dependent kinase 13;
Subtracting from the level of phosphorylation at S183 of Proline-rich AKT1 substrate 1 the level of phosphorylation at T58 of Myc proto-oncogene protein and/or T58 of N-myc proto-oncogene protein;
Subtracting from the level of phosphorylation at T19 of Glycogen synthase kinase-3 alpha the level of phosphorylation at T58 of Myc proto-oncogene protein and/or T58 of N-myc proto-oncogene protein; or
Subtracting from the level of phosphorylation at Y313 of Protein kinase C delta type the level of phosphorylation at T58 of Myc proto-oncogene protein and/or T58 of N-myc proto-oncogene protein.

The method may comprise comparing the levels of phosphorylation of
S183 of Proline-rich AKT1 substrate 1 with S627 of Zinc finger protein 608;
T19 of Glycogen synthase kinase-3 alpha with S627 of Zinc finger protein 608;
T46 of Eukaryotic translation initiation factor 4E-binding protein 2 with S627 of Zinc finger protein 608;
T719 of Signal transducer and activator of transcription 1-alpha/beta with S627 of Zinc finger protein 608;
T719 of Signal transducer and activator of transcription 1-alpha/beta with S1054 of Cyclin-dependent kinase 13;
Y313 of Protein kinase C delta type with S244 of DEP domain-containing mTOR-interacting protein;
Y313 of Protein kinase C delta type with S627 of Zinc finger protein 608;
T719 of Signal transducer and activator of transcription 1-alpha/beta with S780 of Signal transducer and activator of transcription 5A;
S302 of Protein kinase C delta type with S244 of DEP domain-containing mTOR-interacting protein;
T48 of Eukaryotic translation initiation factor 4E-binding protein 2 with S1054 of Cyclin-dependent kinase 13; or
S183 of Proline-rich AKT1 substrate 1 with S1054 of Cyclin-dependent kinase 13.

The method may comprise:
Dividing the level of phosphorylation at S183 of Proline-rich AKT1 substrate 1 by the level of phosphorylation at S627 of Zinc finger protein 608;
Dividing the level of phosphorylation at T19 of Glycogen synthase kinase-3 alpha by the level of phosphorylation at S627 of Zinc finger protein 608;
Dividing the level of phosphorylation at T46 of Eukaryotic translation initiation factor 4E-binding protein 2 by the level of phosphorylation at S627 of Zinc finger protein 608;
Dividing the level of phosphorylation at T719 of Signal transducer and activator of transcription 1-alpha/beta by the level of phosphorylation at S627 of Zinc finger protein 608;
Dividing the level of phosphorylation at T719 of Signal transducer and activator of transcription 1-alpha/beta by the level of phosphorylation at S1054 of Cyclin-dependent kinase 13;
Dvding the level of phosphorylation at Y313 of Protein kinase C delta type by the level of phosphorylation at S244 of DEP domain-containing mTOR-interacting protein;
Dvding the level of phosphorylation at Y313 of Protein kinase C delta type by the level of phosphorylation at S627 of Zinc finger protein 608;
Dvding the level of phosphorylation at T719 of Signal transducer and activator of transcription 1-alpha/beta by the level of phosphorylation at S780 of Signal transducer and activator of transcription 5A;
Dividing the level of phosphorylation at S302 of Protein kinase C delta type by the level of phosphorylation at S244 of DEP domain-containing mTOR-interacting protein;
Dividing the level of phosphorylation at T46 of Eukaryotic translation initiation factor 4E-binding protein 2 by the level of phosphorylation at S1054 of Cyclin-dependent kinase 13; or
Dividing the level of phosphorylation at S183 of Proline-rich AKT1 substrate 1 by the level of phosphorylation at S1054 of Cyclin-dependent kinase 13.

The method may comprise comparing the levels of phosphorylation of
S183 of Proline-rich AKT1 substrate 1 with S627 of Zinc finger protein 608;
T19 of Glycogen synthase kinase-3 alpha with S627 of Zinc finger protein 608;
T46 of Eukaryotic translation initiation factor 4E-binding protein 2 with S627 of Zinc finger protein 608;
T719 of Signal transducer and activator of transcription 1-alpha/beta with S627 of Zinc finger protein 608;
T719 of Signal transducer and activator of transcription 1-alpha/beta with S1054 of Cyclin-dependent kinase 13; or
Y313 of Protein kinase C delta type with S244 of DEP domain-containing mTOR-interacting protein.

The method may comprise:
Dividing the level of phosphorylation at S183 of Proline-rich AKT1 substrate 1 by the level of phosphorylation at S627 of Zinc finger protein 608;
Dividing the level of phosphorylation at T19 of Glycogen synthase kinase-3 alpha by the level of phosphorylation at S627 of Zinc finger protein 608;
Dividing the level of phosphorylation at T46 of Eukaryotic translation initiation factor 4E-binding protein 2 by the level of phosphorylation at S627 of Zinc finger protein 608;
Dividing the level of phosphorylation at T719 of Signal transducer and activator of transcription 1-alpha/beta by the level of phosphorylation at S627 of Zinc finger protein 608;
Dividing the level of phosphorylation at T719 of Signal transducer and activator of transcription 1-alpha/beta by the level of phosphorylation at S1054 of Cyclin-dependent kinase 13; or
Dividing the level of phosphorylation at Y313 of Protein kinase C delta type by the level of phosphorylation at S244 of DEP domain-containing mTOR-interacting protein.

The method may comprise comparing the levels of phosphorylation of
S183 of Proline-rich AKT1 substrate 1 with S627 of Zinc finger protein 608; or
T719 of Signal transducer and activator of transcription 1-alpha/beta with S1054 of Cyclin-dependent kinase 13.

The method may comprise:
Dividing the level of phosphorylation at S183 of Proline-rich AKT1 substrate 1 by the level of phosphorylation at S627 of Zinc finger protein 608; or
Dividing the level of phosphorylation at T719 of Signal transducer and activator of transcription 1-alpha/beta by the level of phosphorylation at S1054 of Cyclin-dependent kinase 13.

Wherein the sample is a peripheral blood sample, the method may comprise any one of:
S183 of Proline-rich AKT1 substrate 1 with S627 of Zinc finger protein 608;
T719 of Signal transducer and activator of transcription 1-alpha/beta with S1054 of Cyclin-dependent kinase 13;
Y313 of Protein kinase C delta type with S244 of DEP domain-containing mTOR-interacting protein;
S302 of Protein kinase C delta type with S244 of DEP domain-containing mTOR-interacting protein; or
T46 of Eukaryotic translation initiation factor 4E-binding protein 2 with S1054 of Cyclin-dependent kinase 13.

Wherein the sample is a bone marrow sample, the method may comprise any one of:
S183 of Proline-rich AKT1 substrate 1 with S627 of Zinc finger protein 608;
T19 of Glycogen synthase kinase-3 alpha with S627 of Zinc finger protein 608;
T48 of Eukaryotic translation initiation factor 4E-binding protein 2 with S627 of Zinc finger protein 608;
T719 of Signal transducer and activator of transcription 1-alpha/beta with S627 of Zinc finger protein 608;
T719 of Signal transducer and activator of transcription 1-alpha/beta with S1054 of Cyclin-dependent kinase 13;
Y313 of Protein kinase C delta type with S627 of Zinc finger protein 608;
T719 of Signal transducer and activator of transcription 1-alpha/beta with S780 of Signal transducer and activator of transcription 5A;
S302 of Protein kinase C delta type with S627 of Zinc finger protein 608;
T19 of Glycogen synthase kinase-3 alpha with S780 of Signal transducer and activator of transcription 5A;
S183 of Proline-rich AKT1 substrate 1 with S780 of Signal transducer and activator of transcription 5A;
Y313 of Protein kinase C delta type with S780 of Signal transducer and activator of transcription 5A;
T719 of Signal transducer and activator of transcription 1-alpha/beta; with T58 of Myc proto-oncogene protein and/or T58 of N-myc proto-oncogene protein; or
S302 of Protein kinase C delta type with S780 of Signal transducer and activator of transcription 5A.

It will be apparent for all of the comparisons by division disclosed herein that equivalent comparisons may be performed where the first biomarker is low or not high in midostaurin responders and the second biomarker is high in midostaurin responders, in which case the comparison may produce a value less than one in predicted midostaurin responders. For comparisons by subtraction the same applies however the comparison may produce a value less than zero in predicted midostaurin responders.

The method may comprise predicting that the acute myeloid leukaemia in the patient may be effectively treated with midostaurin using a multivariate analysis. The multivariate analysis may be orthogonal partial least squares-discrimination analysis (oPLS-DA).

oPLS-DA generates a regression model based on levels of biomarkers (Chong, J., Yamamoto, M. & Xia, J. MetaboAnalystR 2.0: “From Raw Spectra to Biological Insights.” Metabolites 9 (2019). oPLS-DA may be performed using the MetaboAnalyst web-based application (https:/www.metaboanalyst.ca/faces/ModuleView.xhtml).

Alternative multivariate regression methods include, for example, principal component regression (PCR), lasso, ridge regression and elastic net. The multivariate analysis may be selected from the group consisting of orthogonal partial least squares-discrimination analysis (oPLS-DA), PCR, lasso, ridge regression and elastic net.

The method may comprise predicting that the cancer, such as acute myeloid leukaemia, in the patient may be effectively treated with midostaurin using a trained machine learning model. Such a machine learning model may be used to predict the response to midostaurin treatment based on data such as one or more of: the level of one or more proteins, the level of phosphorylation at one or more phosphorylation sites, the sample source (such as from bone marrow or from peripheral blood), the response index, and clinical outcome for a patient.

A machine learning model may be created using various scripts, such as R scripts, to create Support Vector Machine (SVM) models and/or random forest prediction models, for example.

Alternative machine learning algorithms include, for example, artificial neural networks (ANNs), deep learning, logistic regression, GBM, and tree-based algorithms other than RF. The machine learning model may therefore comprise at least one of an SVM model and a random forest prediction model. The skilled person is familiar with machine learning algorithms including SVM models and random forest prediction models, and as such a specific implementation of SVM models and random forest prediction models will now be briefly described.

The SVM models may be created for example using the caret package in R, or using the known and freely-accessible package ClassyFire developed by the Wishart Research Group (http://classyfire.wishartlab.com/), and described in the publication: Djoumbou Feunang Y, Eisner R, Knox C, Chepelev L, Hastings J, Owen G, Fahy E, Steinbeck C, Subramanian S, Bolton E, Greiner R, and Mishart D S. ClassyFire: Automated Chemical Classification With A Comprehensive, Computable Taxonomy. Journal of Cheminformatics, 2016, 8:61. As the skilled person would understand, ClassyFire is a web-based application for automated structural classification of chemical entities. ClassyFire uses an SVM to create the machine learning models. As would be understood, SVM classifies, makes a regression, and creates a novelty detection for the creation of the model. Several such models may be created until the most accurate model is found. Validation of the models is achieved using a validation cohort to estimate the Matthews Correlation Coefficient (MCC) value and assess the accuracy of the prediction, as would be understood. These SVM models output accuracy percentages and MCC values after validation.

The SVM models are trained based on training data. Such data includes “explanatory” data and “response” data for patients in which the treatment outcome is already known. The explanatory data comprises all the data that is used to determine why a patent is or isn't a responder. For example, the explanatory data may be biomarker levels for a particular patient, including the individual levels of each biomarker as obtained from the sample. The response data comprises data indicating whether or not the particular patient actually is or isn't a responder. The training data may therefore comprise a tab-delimited table with a training dataset of patients as columns and biomarkers as rows, and a tab-delimited table with a validation dataset of patients as columns and biomarkers as rows.

The random forest models may be created using the known package randomForest as described in the publication: A. Liaw and M. Wiener (2002). Classification and Regression by randomForest. R News 2(3), 18-22. As the skilled person would understand, this package creates a random forest model based on classification and regression of random forests. The random forest models may be created using random forests and bootstrapping in order to decorrelate the multiple trees generated on different bootstrapped samples from the training data, and then reduce the variance in the trees by averaging. The use of averaging improves the performance of decision trees and avoids overfitting. The randomForest package creates several trees, each one using different variables to create a best version.

The mtry parameter, which is the number of variables available for splitting at each tree node, can be used to define how many variables the data is split into to create different trees. Specifically in this use, the importance of each biomarker in the improvement of the model's accuracy is estimated by defining the biomarker to create a loop, monitoring for a change in accuracy of the model, and defining the best miry based on the biomarker's effect on the accuracy of the model.

The model may then be re-run based on the defined best mtry value. As for the SVM models, a validation cohort is used to estimate the MCC value. These random forest models output accuracy percentages and MCC values after validation, and may also output one or more plots associated with the performance of the model and the importance of each biomarker in the construction of the model.

The random forest models, like the SVM models, are trained using training data. The training data used to train the random forest models may be the same as that used to train the SVM models.

The method may therefore comprise determining the levels of at least two biomarkers and predicting whether a cancer, such as acute myeloid leukaemia, in the patient may be effectively treated with midostaurin, the method comprising inputting the levels of at least two biomarkers into a trained machine learning algorithm, the trained machine learning algorithm being arranged to:
i. Compare the levels of the at least two biomarkers from the sample obtained from the patient with the levels of the same at least two biomarkers from samples obtained from a plurality of patients before administration of midostaurin and data identifying whether for each of the plurality of patients the midostaurin was effective or ineffective, and
ii. Output whether the midostaurin is predicted to be effective or ineffective; optionally wherein the trained machine learning model is a random forests model and/or a Support Vector Machine (SVM) model.

The trained machine learning algorithm may be trained based on training data, the training data comprising:
First data including biomarker levels for a plurality of patients before administration of midostaurin; and
Second data identifying whether for each of the plurality of patients the midostaurin was effective or ineffective.

The levels of the at least two biomarkers may comprise the levels of any of the biomarkers described herein.:

Most proteins are modified in some way by the addition of functional groups and such modifications are effected by protein modifying enzymes. Protein modifications that can be detected by mass spectrometry include phosphorylation, glycosylation, acetylation, methylation and lipidation. These protein modifications have various biological roles in the cell. The modification sites may therefore be sites of post-translational modifications. For example, the modification sites may be sites may be sites of phosphorylation, glycosylation, acetylation, methylation and lipidation. The modification sites are typically protein and/or peptide modification sites. A modification site may be one or more amino acid residues of a peptide or protein to which a functional group such as a phosphate group is added to the peptide or protein. Alternative functional groups include carbohydrates, acetyl groups, methyl groups and lipids. By “protein modifying enzyme” is therefore meant an enzyme which catalyses a reaction involving the addition of a functional group to a protein or peptide. A “modified peptide” is defined herein as a peptide which has been modified by the addition or removal of a functional group.

A “protein modifying enzyme” is defined herein as an enzyme which catalyses a reaction involving the addition or removal of a functional group to a protein or peptide.

A “peptide” as defined herein is a short amino acid sequence and includes oligopeptides and polypeptides. Typically, such peptides are between about 5 and 30 amino acids long, for example from 6 or 7 to 25, 26 or 27 amino acids, from 8, 9 or 10 to 20 amino acids, from 11 or 12 to 18 amino acids or from 14 to 16 amino acids, for example 15 amino acids. However, shorter and longer peptides, such as between about 2 and about 50, for example from about 3 to about 35 or 40 or from about 4 to about 45 amino acids can also be used. Typically, the peptide is suitable for mass spectrometric analysis, that is the length of the peptide is such that the peptide is suitable for mass spectrometric analysis. The length of the peptide that can be analysed is limited by the ability of the mass spectrometer to sequence such long peptides. In certain cases polypeptides of up to 300 amino acids can be analysed, for example from 50 to 250 amino acids, from 100 to 200 amino acids or from 150 to 175 amino acids.

As described herein, the methods may be based on the analysis of peptides and/or modified peptides which are identified and/or quantified using MS-based techniques. In some embodiments, the method of the invention therefore includes a step of identifying and/or quantifying peptides and/or modified peptides in a sample using mass spectrometry (MS).

The method may be based on the analysis of phosphorylated peptides. Phosphorylated peptides contain one or more amino acid which is phosphorylated (i.e. a phosphate (PO₄) group has been added to that amino acid). Such phosphorylated amino acids are referred to herein as “phosphorylation sites”. When a peptide is phosphorylated by a particular protein kinase, it is referred to as a “substrate” of that protein kinase. In relation to this embodiment of the invention, the term “phosphoprotein” is used herein to refer to a phosphorylated protein and the term “phosphopeptide” is used herein to refer to a phosphorylated peptide. Kinases of particular interest in the context of the present disclosure are those involved in the FLT3/PKC pathway.

As demonstrated by the experimental data provided herein, the inventors have found that the present invention provides an accurate test for identifying cancer patients who will be responsive to treatment with midostaurin, which is a FLT3/PKC pathway inhibitor. In embodiments of the invention the cancer is acute myeloid leukemia (AML). However, it will be appreciated that the cancer may be selected from any one of: acute myeloid leukemia (AML); high-risk myelodysplastic syndrome (MDS); aggressive systemic mastocytosis (ASM): systemic mastocytosis with associated hematological neoplasm (SM-AHN); and mast cell leukemia (MCL).

The sample used in the methods of the invention can be any sample which contains peptides from a patient. The patient may be a human or animal suffering from or suspected of suffering from acute myeloid leukaemia. Where the method involves control samples these may or may not be from a human or animal suffering from or suspected of suffering from cancer (the control sample may be from a healthy individual). The invention thus encompasses the use of samples obtained from human and non-human sources.

Samples are typically obtained prior to the methods of the invention being performed. The methods of the invention are in vitro methods accordingly. In some alternative embodiments, the method may further comprise a step or steps of sample collection.

As used herein, the term “patient” and the term “subject” are used interchangeably. The patient may or may not have received any previous treatment for cancer. The patient may be termed an “individual” patient.

The present invention finds use in the field of personalized medicine. Typically, therefore, the biological sample is derived from a human, and can be, for example, a sample of a bodily fluid such as bone marrow or blood, or another tissue. Typically, the biological sample is from a tissue, typically a primary tissue, or from a tissue which has undergone processing after isolation such as culturing of cells, such as leukemia cells, or storage. For example, the sample can be a tissue from a human or animal. The human or animal can be healthy or diseased. In an embodiment, the human has been diagnosed with or is suspected as having a cancer, such as acute myeloid leukemia (AML). Suitably, the sample comprises leukemia cells. The leukemia cells may be myeloblasts, abnormal red blood cells or platelets. Accordingly, the tissue may be from a peripheral blood sample or from a bone marrow sample. The sample may be a peripheral blood sample or a bone marrow sample. The sample may be leukaemia cells which have previously been obtained from the patient. This invention is applicable to all AML patients, including newly-diagnosed (untreated) AML patients, AML patients who have undergone or are undergoing other forms of treatment, and relapsed AML patients. The AML patient may be newly diagnosed. The patient may be newly diagnosed with AML that is FLT3 mutation positive or negative. The patient may be newly diagnosed with AML based on analysis of the sample used in the method of the invention. The patient may be newly diagnosed with AML based on analysis of an aliquot or portion of the sample used in the method of the invention. The patient may be newly diagnosed with AML based on analysis of a second sample obtained at the same or at a similar time as the sample used in the method of the invention. The sample may have been obtained prior to diagnosis of AML and/or prior to treatment for AML. The sample may have been obtained after diagnosis of AML and/or after treatment for AML.

Acute myeloid leukaemia (AML), also known as acute myelogenous leukaemia, acute myeloblastic leukaemia, acute granulocytic leukemia or acute nonlymphocytic leukemia, is an aggressive cancer of the blood and bone marrow. AML is characterised by excessive production of immature white blood cells, known as myeloblasts, by bone marrow. In healthy individuals, blasts normally develop into mature white blood cells. In AML, however, the blasts do not differentiate normally but remain at a premature arrested state of development.

In AML, the bone marrow may also make abnormal red blood cells and platelets. The number of these abnormal cells increases rapidly, and the abnormal cells begin to crowd out the normal white blood cells, red blood cells and platelets that the body needs. If left untreated, acute myeloid leukaemia is rapidly fatal.

Various classification systems have been devised for classifying AML into disease subtypes, with the aim of enabling more accurate prognosis of disease progression and identification of the optimal form of treatment. The earliest system was the French-American-British (FAB) classification, first devised in the 1970s by a group of French, American and British leukaemia experts. This system divides AML into subtypes according to the type of cell from which the leukaemia has developed, and the stage of maturity reached by the myeloblast cells at the point of arrest. Subtypes M0 to M5 originate from precursors of white blood cells and range from undifferentiated myeloblastic leukaemia (M0) to monocytic leukaemia (M5). Subtype M6 originates in very early forms of red blood cells (erythroid leukaemia), whilst subtype M7 AML starts in early forms of cells that form platelets (megakaryoblastic leukaemia).

Under the FAB system, AML is categorised by visual inspection of cytomorphological features under the microscope, and by identification of various chromosomal abnormalities. An updated version of the FAB categorisation was published in 1985—see Bennett et al, Proposed revised criteria for the classification of acute myeloid leukaemia, Ann Intern Med 1985; 103(4): 620-625.

Since the FAB system was first devised in the 1970s, the level of knowledge in the field has moved on considerably. Whilst the system has been updated to incorporate some of this knowledge, it was felt to be necessary to create a new classification system, taking into account additional factors now known to affect prognosis and to be determinative in optimising effective treatment.

The World Health Organization (WHO) classification system accordingly divides AML into several broad groups. These include:
AML with recurrent genetic abnormalities, meaning with specific chromosomal changes
AML with multilineage dysplasia
AML, related to previous therapy that is damaging to cells, including chemotherapy and radiotherapy, also called therapy-related myeloid neoplasm
AML that is not otherwise categorized—including:
   Undifferentiated AML (M0)
   AML with minimal maturation (M1)
   AML with maturation (M2)
   Acute myelomonocytic leukemia (M4)
   Acute monocytic leukemia (M5)
   Acute erythroid leukemia (M6)
   Acute megakaryoblastic leukemia (M7)
   Acute basophilic leukemia
   Acute panmyelosis with fibrosis
   Myeloid sarcoma (also known as granulocytic sarcoma or chloroma)

In addition to these two main classification systems, AML is further categorised and subtyped by reference to specific molecular markers which are found to correlate with certain phenotypes and outcomes. For example, patients with mutations in the NPM1 gene or CEBPA genes are known to have a better long term outcome, whilst patients with certain mutations in FLT3 have been found to have a worse prognosis— see Yohe et al, J Clin Med. 2015 March 4(3): 460-478.

The AML may be FLT3 mutation positive. The patient may have a mutation in the FLT3 gene. The patient may have an activating mutation in the FLT3 gene. An activating mutation of FLT3 is a mutation which has the effect of constitutively switching the FLT3 protein “on”. Such mutations may, for example, include internal tandem duplications (ITD) of the juxtamembrane domain or point mutations usually involving the tyrosine kinase domain, such as at D835.

In particular embodiments, the method may further comprise performing an in vitro assay to detect the genotype of leukaemia cells in the sample obtained from the patient and determining that FLT3 in the leukaemia cells has an activating mutation; and/or performing an assay to detect the expression or activation in the leukaemia cells in the sample obtained from the patient of one or more activity markers of a FLT-3 driven signalling pathway that is involved in cell proliferation or cell survival other than the RAS-RAF-MEK-ERK pathway, such as the PKC pathway, the PI3K-AKT-MTOR-S6K pathway, the PAK pathway, the JAK-STAT pathway, or the CAMKK pathway, and determining that the FLT3-driven kinase signalling pathway is activated in the leukaemia cells; and/or performing an assay to detect the level of phosphorylation of one or both of TOP2A and/or KDMSC in the leukaemia cells in the sample obtained from the patient and determining that TOP2A and/or KDMSC are phosphorylated or are phosphorylated at a high level in the leukaemia cells.

The AML may be FLT3 mutation negative. The patient may not have a mutation in the FLT3 gene. The patient may not have an activating mutation in the FLT3 gene. Indeed, it is a surprising finding by the present inventors that the novel proteomic and phosphoproteomic signatures described herein are able to identify candidate subjects who are likely to respond or to not-respond to midostaurin treatment irrespective of their FL3T mutation status.

The method may further comprise performing an in vitro assay to detect the genotype of leukaemia cells in the sample obtained from the patient and determining that FLT3 in the leukaemia cells does not have an activating mutation; and/or performing an assay to detect the expression or activation in the leukaemia cells in the sample obtained from the patient of one or more activity markers of a FLT-3 driven signalling pathway that is involved in cell proliferation or cell survival other than the RAS-RAF-MEK-ERK pathway, such as the PKC pathway, the PI3K-AKT-MTOR-S6K pathway, the PAK pathway, the JAK-STAT pathway, or the CAMKK pathway, and determining that the FLT3-driven kinase signalling pathway is not activated in the leukaemia cells; and/or performing an assay to detect the level of phosphorylation of one or both of TOP2A and/or KDMSC in the leukaemia cells in the sample obtained from the patient and determining that TOP2A and/or KDMSC are not phosphorylated or are phosphorylated at a low level in the leukaemia cells.

“Determining” according to the methods of the invention may comprise performing an in vitro assay. Step (a) of the method may comprise performing an in vitro assay to detect the level one or more proteins and/or the level of phosphorylation at the one or more phosphorylation sites in the sample obtained from the patient. Said assay may be an LC-MS/MS assay or an assay based on affinity reagents such as aptamers, molecularly imprinted polymers, or antibodies (immunochemical assays). The assay based on affinity reagents may be a Western blot assay, an ELISA assay or a reversed phase protein assay. The assay can be carried out by any method involving mass spectrometry (MS), such as liquid chromatography-mass spectrometry (LC-MS). The LC-MS or LC-MS/MS is typically label-free MS but techniques that use isotope labelling as the basis for to detecting the level one or more proteins and/or the level of phosphorylation at the one or more phosphorylation sites can also be used as the basis for the analysis. The assay may be an LC-MS/MS assay. The assay may comprise using a label-free mass spectrometry approach as previously described in Casado et al., 2018 Leukemia 32, 1818-1822 and/or WO 2018/234404, both of which are incorporated by reference herein in their entirety.

Peptides can be obtained from the sample using any suitable method known in the art. In one embodiment, prior to step (a), the method of the invention comprises:
(1) lysing cells in the sample;
(2) extracting the proteins from the lysed cells obtained in step (1); and
(3) cleaving said proteins into peptides.

In step (1) of this embodiment of the invention, the cells in the sample are lysed, or split open. The cells can be lysed using any suitable means known in the art, for example using physical methods such as mechanical lysis (for example using a Waring blender), liquid homogenization, sonication or manual lysis (for example using a pestle and mortar) or detergent-based methods such as CHAPS or Triton-X. Typically, the cells are lysed using a denaturing buffer such as a urea-based buffer.

In step (2) of this embodiment of the invention, proteins are extracted from the lysed cells obtained in step (1). In other words, the proteins are separated from the other components of the lysed cells.

In step (3) of this embodiment of the invention, the proteins from the lysed cells are cleaved into peptides. In other words, the proteins are broken down into shorter peptides. Protein breakdown is also commonly referred to as digestion. Protein cleavage can be carried out in the present invention using any suitable agent known in the art.

Protein cleavage or digestion is typically carried out using a protease. Any suitable protease can be used in the present invention. In the present invention, the protease is typically trypsin, chymotrypsin, Arg-C, pepsin, V8, Lys-C, Asp-C and/orAspN. Alternatively, the proteins can be cleaved chemically, for example using hydroxylamine, formic acid, cyanogen bromide, BNPS-skatole, 2-nitro-5-thiocyanobenzoic acid (NTCB) or any other suitable agent.

Peptides (including phosphorylated peptides) detected by carrying out liquid chromatography-tandem mass spectrometry (LC-MS/MS) may be compared to a known reference database in order to identify the peptides (including phosphorylated peptides). This step is typically carried out using a commercially available search engine, such as, but not restricted to, the MASCOT, ProteinProspector, Andromeda, or Sequest search engines. Other computer programmes and workflows, such as MaxQuant [Nature Biotechnology 26, 1367-1372 (2008)] may be used to quantify peptides.

The computer program named PESCAL (Cutillas and Vanhaesebroeck, Molecular & Cellular Proteomics 6, 1560-1573 (2007)) automates the quantification of each peptide (including phosphorylated peptides) listed in the database in LC-MS runs of modified peptides (including phosphorylated peptides) taken from biological experiments. For these biological experiments, proteins in cell lysates are digested using trypsin or other suitable proteases. Peptide (such as phosphopeptide) internal standards, which are reference modified peptides (including reference phosphorylated peptides), are spiked at known amounts in all the samples to be compared. Peptides (including phosphorylated peptides) in the resultant peptide mixture may be enriched using a simple-to-perform IMAC or TiO₂ extraction step. Enriched peptides (including phosphorylated peptides) are analysed in a single LC-MS run of typically but not restricted to about 120 minutes (total cycle).

PESCAL then constructs extracted ion chromatograms (XIC, i.e, an elution profile) for each of the peptides (including phosphorylated peptides) present in the database across all the samples that are to be compared. The program also calculates the peak height and area under the curve of each XIC.

The data is normalised by dividing the intensity reading (peak areas or heights) of each peptide (including phosphopeptide) analyte by those of the peptide (including phosphopeptide) ISs.

Quantification of modifications such as phosphorylation can also be carried out using MS techniques that use isotope labels for quantification, such as metabolic labeling (e.g., stable isotope labeled amino acids in culture, (SILAC); Olsen, J. V. et al. Cell 127, 635-648 (2006)), and chemical derivatization (e.g., iTRAQ (Ross, P. L.; et al. Mol Cell Proteomics 2004, 3, (12), 1154-69), ICAT (Gygi, S. P. et al. Nat Biotechnol 17, 994-999 (1999)), TMT (Dayon L et al, Anal Chem. 2008 Apr. 15; 80(8):2921-31) techniques. Protein modifications can be quantified with LC-MS techniques that measure the intensities of the unfragmented ions or with LC-MS/MS techniques that measure the intensities of fragment ions (such as Selected Reaction Monitoring (SRM), also named multiple reaction monitoring (MRM) and parallel-reaction monitoring (PRM)).

Other computer programs such as Skyline are alternatives to PESCAL.

The method may therefore comprise normalising the level of each biomarker to the level of an internal standard, such as an isotopically labelled standard. This may provide absolute quantification of the level of the biomarker.

LC-MS/MS may be suitable for use in situations where there is access to the equipment required in, for example, in hospital or in centralized laboratories. However, more conveniently, the levels of the at least one biomarker in the samples may be measured using assays based on affinity reagents such as immunoassays. Immunoassays have the potential to be miniaturised to run on a microfluidics device or test-strip and may be more suited for clinical point-of-care applications. Embodiments of the invention which incorporate an immunoassay may therefore be used in situ by a primary healthcare provider for assistance in prescribing a statin for an individual patient.

The levels of the at least one biomarker may be measured using a homogeneous or heterogeneous immunoassay.

Thus, in some embodiments, the levels of the or each biomarker may be measured in solution by binding to labelled antibodies, aptamers or molecular imprinted polymers that are present in excess, whereby binding alters detectable properties of the label. The amount of a specific biomarker present will therefore affect the amount of the label with a particular detectable property. As is well known in the art, the label may comprise a radioactive label, a fluorescent label or an enzyme having a chromogenic or chemiluminescent substrate that is coloured or caused or allowed to fluoresce when acted on by the enzyme.

The antibodies may be polyclonal or monoclonal with specificity for the biomarker. In some embodiments, monoclonal antibodies may be used.

Alternatively, a heterogeneous format may be used in which the at least one biomarker is captured by surface-bound antibodies for separation and quantification. In some embodiments, a sandwich assay may be used in which a surface-bound biomarker is quantified by binding a labelled secondary antibody.

Suitably, the immunoassay may comprise an enzyme immunoassay (EIA) in which the label is an enzyme such, for example, as horseradish peroxidase (HRP). Suitable substrates for HRP are well known in the art and include, for example, ABTS, OPD, AmplexRed, DAB, AEC, TMB, homovanillic acid and luminol. In some embodiments, an ELISA immunoassay may be used; a sandwich ELISA assay may be particularly preferred.

The immunoassay may be competitive or non-competitive. Thus, in some embodiments, the amounts of the at least one biomarker may be measured directly by a homogeneous or heterogeneous method, as described above. Alternatively, the biomarker in the samples may be sequestered in solution with a known amount of antibody which is present in excess, and the amount of antibody remaining then determined by binding to surface-bound biomarker to give an indirect read-out of the amount of biomarker in the original sample. In another variant, the at least one biomarker may be caused to compete for binding to a surface bound antibody with a known amount of a labelled biomarker.

The surface bound antibodies or biomarker may be immobilised on any suitable surface of the kind known in the art. For instance, the antibodies or biomarker may be immobilised on a surface of a well or plate or on the surface of a plurality of magnetic or non-magnetic beads.

In some embodiments, the immunoassay may be a competitive assay, further comprising a known amount of the biomarker, which is the same as the one to be quantitated in the sample, but tagged with a detectable label. The labelled biomarker may be affinity-bound to a suitable surface by an antibody to the biomarker. Upon adding the sample a proportion of the labelled biomarker may be displaced from the surface-bound antibodies, thereby providing a measure of the level of biomarker in the sample.

In some embodiments, the immunoassay may comprise surface-bound biomarker, which is the same as the biomarker that is to be quantitated in the sample, and a known amount of antibodies to the biomarker in solution in excess. The sample is first mixed with the antibodies in solution such that a proportion of the antibodies bind with the biomarker in the sample. The amount of unbound antibodies remaining can then be measured by binding to the surface-bound biomarker.

In some embodiments, the immunoassay may comprise a labelled secondary antibody to the biomarker or to a primary antibody to the biomarker for quantifying the amount of the biomarker bound to surface-bound antibodies or the amount of primary antibody bound to the biomarker immobilised on a surface.

Measuring biomarker levels may be by equipment for measuring the level of a specific biomarker in a sample comprising a sample collection device and an immunoassay. The equipment may further comprise a detector for detecting labelled biomarker or labelled antibodies to the biomarker in the immunoassay. Suitable labels are mentioned above, but in a preferred embodiment, the label may be an enzyme having a chromogenic or chemiluminescent substrate that is coloured or caused or allowed to fluoresce when acted on by the enzyme.

The immunoassay or equipment may be incorporated into a miniaturised device for measuring the level of at least one biomarker in a biological sample. Suitably, the device may comprise a lab-on-a-chip.

Measuring biomarker levels may be by a device for measuring the level of at least one biomarker in a sample obtained from a patient, the device comprising one or more parts defining an internal channel having an inlet port and a reaction zone, in which a biomarker in a sample may be reacted with an immobilised primary antibody for the biomarker for capturing the biomarker, or a primary antibody for the biomarker in excess in solution after mixing with the sample upstream of the reaction zone may be reacted with biomarker, which is the same as the one to be measured in the sample, but immobilised on a surface within the reaction zone, for quantifying directly or indirectly the amount of the biomarker in the sample.

The captured biomarker or primary antibody may then be detected using a secondary antibody to the biomarker or primary antibody, which is tagged with an enzyme.

As described above, the enzyme may have a chromogenic or chemiluminescent substrate that is coloured or caused or allowed to fluoresce when acted on by the enzyme. Suitably, the one or more parts of the device defining the channel, at least adjacent the reaction zone, may be transparent to light, at least in a range of wavelengths encompassing the colour or fluorescence of the substrate to allow detection of a reaction between the biomarker or primary antibody and the secondary antibody using a suitable detector such, for example, as a photodiode, positioned outside the channel or further channel.

In some embodiments, the device may comprise a plurality of channels, each with its own inlet port, for measuring the levels of a plurality of different biomarkers in the sample in parallel. Therefore, each channel may include a different respective immobilised primary antibody or biomarker. Suitably, the device may comprise one or more selectively operable valves associated with the one or more inlet ports for controlling the admission of a sequence of different reagents into to the channels such, for example, as the sample, wash solutions, primary antibody, secondary antibody and enzyme substrate.

The device therefore may comprise a microfluidics device. The channel may include a reaction zone. Microfluidics devices are known to those skilled in the art. A review of microfluidic immunoassays or protein diagnostic chip microarrays is provided by Chin et al. 2012. Lab on a Chip. 2012; 12:2118-2134. A microfluidics device suitable for carrying out an ELISA immunoassay at a point-of-care is disclosed by Chan C D, Laksanasopin T, Cheung Y K, Steinmiller D et al. “Microfluidics-based diagnostics of infectious diseases in the developing world”. Nature Mediane. 2011; 17(8):1015-1019, the contents of which are incorporated herein by reference.

Midostaurin is a staurosporine analogue also referred to as 4′-N-Benzoylstaurosporine, and has a full chemical name of N-[(9S,I0R,I IR,I3R)-2,3,I0,I I,I2,I3-hexahydro-I0-methoxy-9-methyl-I-oxo-9,I3-epoxy-IH, 9H-diindolo[1,2,3-gh: 3′,2′,I′-Im]pyrrolo[3,4-j][I, 7]benzodiazonin-I I-yl]-N-methylbenzamide. As a drug it is used as anti-tumour agent and is sold under the brand names Rydapt® and Tauritmo®. It is approved by the US FDA for the treatment of adult patients with newly diagnosed acute myeloid leukemia (AML) who have a specific genetic mutation in FLT3, in combination with chemotherapy. The drug is approved for use with a companion diagnostic, the LeukoStrat CDx FLT3 Mutation Assay, which is used to detect the FLT3 mutation in patients having been diagnosed with AML. Preparation of midostaurin is described, for example, in U.S. Pat. No. 5,093,330 and also in International patent Application published as WO-A-2019/215,759.

Midostauin inhibits multiple receptor tyrosine kinases, including FLT3 and KIT kinase. Midostaurin inhibits FLT3 receptor signalling and induces cell cycle arrest and apoptosis in leukaemic cells expressing FLT3 ITD or TKD mutant receptors or over-expressing FLT3 wild type receptors. In vitro data indicate that midostaurin inhibits D816V mutant KIT receptors at exposure levels achieved in patients (average achieved exposure higher than IC50). In vitro data indicate that KIT wild type receptors are inhibited to a much lesser extent at these concentrations (average achieved exposure lower than IC50). Midostaurin interferes with aberrant KIT D816V-mediated signalling and inhibits mast cell proliferation, survival and histamine release. In addition, midostaurin inhibits several other receptor tyrosine kinases such as PDGFR (platelet-derived growth factor receptor) or VEGFR2 (vascular endothelial growth factor receptor 2), as well as members of the serine/threonine kinase family PKC (protein kinase C). Midostaurin binds to the catalytic domain of these kinases and inhibits the mitogenic signalling of the respective growth factors in cells, resulting in growth arrest. Midostaurin in combination with chemotherapeutic agents (cytarabine, doxorubicin, idarubicin and daunorubicin) resulted in synergistic growth inhibition in FLT3-ITD expressing AML cell lines.

In an alternative formulation of the first aspect, the invention provides a method for predicting the efficacy of midostaurin for treatment of acute myeloid leukaemia in a patient, comprising the steps of analysing data relating to the level in a sample from the patient of one or more proteins described herein in reference to proteomic signatures, and/or biomarkers as described herein in reference to phosphoproteomic signatures.

Said data may, for example, include any type of data obtained from an assay measuring protein expression or phosphorylation, such as by mass spectrometry, mass cytometry or any other technique or assay that is known in the art. In some embodiments, said data has previously been recorded and step (a) comprises obtaining said data for analysis. In other embodiments, step (a) further comprises collecting and recording said data for analysis, according to standard conventional methods and protocols known in the art, for example by mass spectrometry or mass cytometry.

The method may further comprise analysing data relating to the mutational status of FLT3 in the sample, or in leukaemia cells obtained from the patient. The method may comprise determining the mutational status of FLT3 in leukaemia cells obtained from the patient by analysing data relating to the genotype of said leukaemia cells and/or determining the activation in the leukaemia cells of a FLT-3 driven kinase signalling pathway. Said data relating to the genotype of the leukaemia cells may comprise any information from which a skilled person could deduce the presence or absence of an activating mutation in FLT3. The data may include, without limitation, the sequence of the FLT3 gene in the leukaemia cells, the sequence of the FLT3 protein expressed by the leukaemia cells, or data recording the presence or absence of an activating mutation in FLT3 in the leukaemia cells. Said data may be gathered and interpreted by the skilled person without difficulty according to techniques and protocols well known in the art.

According to further aspect, the invention provides a method of screening a plurality of patients with acute myeloid leukaemia to determine whether the acute myeloid leukaemia of any one or more of said plurality of patients may be effectively treated with midostaurin, the screening methods substantially as described above.

As used herein, references to midostaurin include midostaurin treatment as monotherapy as well as part of a combination therapy, such as in combination with chemotherapy. As used herein, references to “midostaurin” may therefore be substituted for “a combination therapy comprising midostaurin”, “midostaurin and chemotherapy” or “a combination treatment comprising midostaurin and chemotherapy”. The method may be a method of screening a plurality of patients with acute myeloid leukaemia to determine whether the acute myeloid leukaemia of any one or more of said plurality of patients may be effectively treated with midostaurin and chemotherapy. The chemotherapy may be cytarabine, doxorubicin, idarubicin and/or daunorubicin. The chemotherapy may be daunorubicin & cytarabine. The chemotherapy may be cytarabine.

According to another aspect, the invention provides a computer implemented method for predicting the efficacy of midostaurin for treatment of acute myeloid leukaemia in a patient, comprising performing any one of the methods described in detail herein.:

The method may comprise
(a) receiving in a computer data identifying a patient who is suffering from acute myeloid leukaemia and data representing:
   (i) the level in a sample from the patient of one or more proteins set out in the proteomic signatures described above and/or
   (ii) the level in a sample from the patient of phosphorylation at one or more phosphorylation sites selected from the phosphoproteomic signatures described above.

The methods of the invention may therefore be implemented on a computer, using a computer program product. The computer program product may include computer code arranged to instruct a computer to perform the functions of one or more of the various methods described above. The computer program and/or the code for performing such methods may be provided to an apparatus, such as a computer, on a computer readable medium or computer program product. The computer readable medium may be transitory or non-transitory. The computer readable medium could be, for example, an electronic, magnetic, optical, electromagnetic, infrared, or semiconductor system, or a propagation medium for data transmission, for example for downloading the code over the Internet. Alternatively, the computer readable medium could take the form of a physical computer readable medium such as semiconductor or solid-state memory, magnetic tape, a removable computer diskette, a random access memory (RAM), a read-only memory (ROM), a rigid magnetic disc, and an optical disk, such as a CD-ROM, CD-R/W or DVD.

An apparatus such as a computer may be configured in accordance with such code to perform one or more processes in accordance with the various methods discussed herein. In one arrangement the apparatus comprises a processor, memory, and a display. Typically, these are connected to a central bus structure, the display being connected via a display adapter. The system can also comprise one or more input devices (such as a mouse and/or keyboard) and/or a communications adapter for connecting the apparatus to other apparatus or networks. Such an apparatus may take the form of a data processing system. Such a data processing system may be a distributed system. For example, such a data processing system may be distributed across a network.

The midostaurin is preferably administered to a patient in a “therapeutically effective amount”, this being sufficient to show benefit to the patient and/or to ameliorate, eliminate or prevent one or more symptoms of a disease. As used herein, “treatment” includes any regime that can benefit the patient.

Midostaurin may be used in combination with standard daunorubicin and cytarabine induction and high-dose cytarabine consolidation chemotherapy. Midostaurin may be used for patients in complete response followed by midostaurin single agent maintenance therapy. Midostaurin may be used for adult patients with newly diagnosed acute myeloid leukaemia (AML) who are FLT3 mutation-positive.

Dosages of midostaurin for use in the present invention can vary between wide limits, depending upon the stage of the AML, the age and condition of the individual to be treated, etc. and a physician will ultimately determine appropriate dosages to be used. This dosage can be repeated as often as appropriate. If side effects develop the amount and/or frequency of the dosage can be reduced, in accordance with normal clinical practice.

The midostaurin may be administered as 25 mg soft capsules. The midostaurin may be taken orally twice daily at approximately 12-hour intervals. The capsules may be taken with food. Prophylactic antiemetics may be co-administered in accordance with local medical practice as per patient tolerance.

The midostaurin may be administered as 50 mg orally twice daily. The midostaurin may be administered on days 8-21 of induction and consolidation chemotherapy cycles, and then for patients in complete response every day as single agent maintenance therapy until relapse for up to 12 cycles of 28 days each.

The midostaurin may be administered as 100 mg orally twice daily.

Hence, embodiments of the present invention may provide dosage regimen methods of treatment that comprise administering midostaurin to a patient with cancer, wherein the the patient has been predicted to be effectively treated with midostaurin according to the predictive approaches described herein.

If a patient is classified ahead of treatment as a predicted midostaurin non-responder then a clinician may treat that patient differently to a patient classified as a predicted midostaurin responder.

Classifying the patient as a predicted midostaurin non-responder or as a predicted midostaurin responder may allow the adoption of a particular, or an alternative, treatment regime more suited to the patient.

The term “classifying” is used interchangeably with the terms, “diagnosing” and “predicting”. The method may be considered a method for diagnosing whether a patient having, suspected of having, or at risk of developing acute myeloid leukaemia will respond to treatment with midostaurin, accordingly.

The method may further comprise selecting a treatment for the patient. The treatment for the patient may be selected on the basis of the classification of the patient as a predicted midostaurin non-responder or as a predicted midostaurin responder.

The method may further comprise selecting a treatment for the patient wherein:
(a) if the patient is a predicted midostaurin responder then midostaurin is selected, and
(b) if the patient is a predicted midostaurin non-responder then a treatment other than midostaurin is selected;
and optionally further comprising administering the selected treatment to the patient.

The method may further comprise administering the treatment selected for the patient.

Preferred features for the second and subsequent aspects of the invention are as for the first aspect of the invention mutatis mutandis. It will be appreciated that all embodiments described herein are considered to be broadly applicable and combinable with any and all other consistent embodiments, as appropriate. Such combinations are considered to fall within the scope of the present invention.

The invention may be implemented by developing research use only assays based on affinity reagents (such as immunochemical) or mass spectrometry and then seek regulatory approval for CDx. These assays may measure the signatures as single biomarkers or in a multiplexed manner.

The median may be used as a cut-off for deciding whether the marker has ‘high’ or ‘low’ expression in the samples. The reason to use ratios of markers is that this improves usefulness in the clinic. One of the pair members is increased in sensitive cells and the other is increased in resistant cells. By taking the ratio of the two, one does not need to compare to an internal standard because the comparison is between endogenous proteins that are present in the sample. A final assay could report an index of expression of the marker increased in sensitive cells (say AKT1 S183) relative to the marker increased in resistant cells (say ZNF608 S627). This index could then be calibrated such that a high value (above a certain threshold) indicates that patients are ‘positive’ for the assay and so they are likely to be sensitive to treatment with midostaurin.

Another approach is to measure all the markers disclosed herein, normalize them against each other (by for example scaling them to the median expression) and then feed them to a pre-trained predictive model (e.g., by machine learning). The output of the model is then either ‘positive’, in which case patients should be treated with the drug, or ‘negative’, meaning that patients should receive an alternative treatment.

The assay may involve the use of internal standards, such as isotopically labelled standards for absolute quantification. This approach may provide an assay that is more robust. The assay optimally involves adding internal standards, and that these standards are optionally isotopically peptides with the same sequence as the target analytes.

Further advantages of the invention are described below.

The presently disclosed signatures have higher predictive power than currently used companion diagnostics (CDx) for midostaurin. The invention therefore provides an advance in precision diagnostics and personalised medicine of AML.

The invention opens the possibility of using midostaurin to treat as subpopulation of FLT3 mutant-negative patients, who are currently ineligible and who represent 70% of all AML cases.

The presently disclosed signatures may be incorporated into research use and also into CDx tests to help the pharmaceuticals industry select patients for inclusion in clinical trials. The CDx could be used by clinicians to routinely decide treatment options.

The use of biomarker ratios or biomarker combinations has the advantage that the clinical assay developed from these biomarkers can be internally normalized.

The inventors have identified phospho-signatures with the potential to further stratify FLT3 mutant-positive (FLT3+) AML for midostaurin treatment Other variables were considered (e.g. age, transplant, karyotype) and none correlated with response to midostaurin+ chemo. Analysis has also been performed on FLT3 mutant-negative cases to validate signatures in this group. The presence of PRKCD signalling components in signatures provides a rationale for midostaurin activity in sensitive cases.

Rationale for the use of direct readouts of kinase activity (such as phospho-markers) to stratify patients for therapy in order to increase the number of patients that may be treated and benefit from therapies that target kinase signaling.

Overall, these analyses uncovered a range of phosphorylation sites, proteins and computer models that stratify patients based on their clinical responses to midostaurin plus chemotherapy. These molecular markers, by themselves or in combination, and algorithms could be used to predict patients more likely to respond to this treatment.

The present invention will now be described by way of reference to the following Examples and accompanying Drawings which are present for the purposes of illustration only and are not to be construed as being limiting on the invention.

### Example 1—Overview of Study and Clinical Sample Set

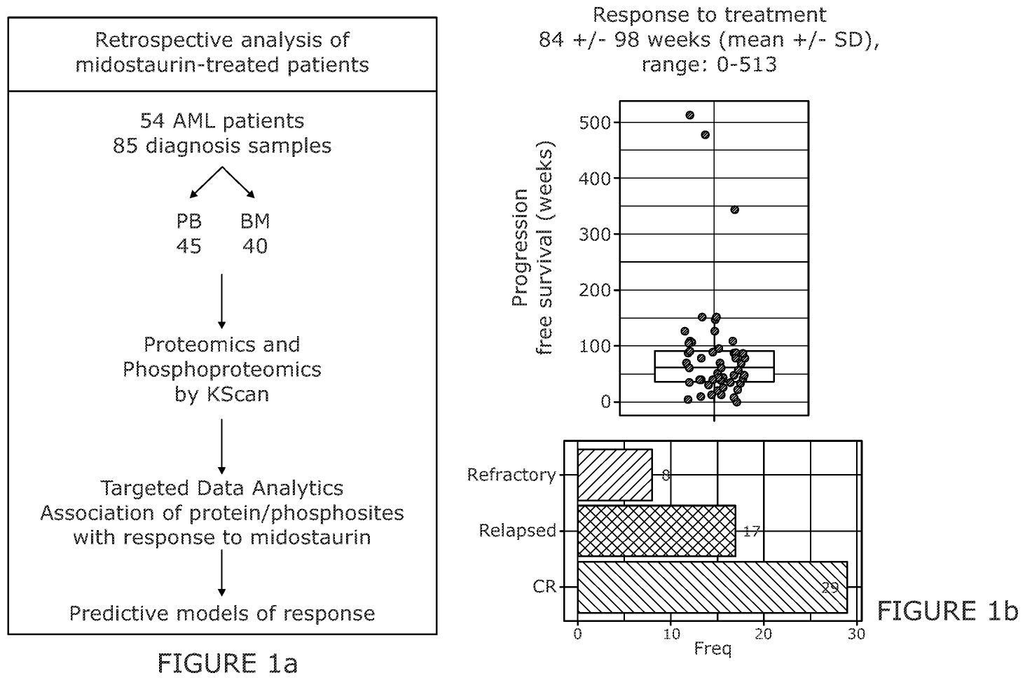

The study design, summarised in FIG. 1a, aimed at identifying proteomic and phosphoproteomic signatures that predict response to midostaurin plus chemotherapy in FLT3 mutant positive AML. We obtained 85 samples at diagnosis from 54 AML patients who were subsequently treated with midostaurin plus chemotherapy at the Princess Margaret Hospital as part of their clinical treatment. These samples were from bone marrow (BM, n=40) or peripheral blood (PB, n=45) cells. Responses to midostaurin were 84 weeks on average (0-513 range) and 25 patients were refractory or relapsed at the time of carrying out the analysis (Figure ib). We carried out proteomic and phosphoproteomics analysis of these samples using label-free mass spectrometry approaches as previously described (Casado et al., 2018 Leukemia 32, 1818-1822). This led to the identification and quantification of 9,963 phosphopeptides and 51,385 unmodified peptides belonging to 7,716 proteins (Table 7).

Patients were all FTL3 mutant-positive with 44 of them having internal tandem duplications (ITDs) and 12 of them mutations in the tyrosine kinase domain (TKD). Two patients had both ITD and TKD mutations.

TABLE 7

| Proteomics | | Phosphoproteomics | |
|---|---|---|---|
| identification summary | | identification summary | |
| parameter | value | parameter | value |
| Number of peptides | 61159 | Number of peptides | 14671 |
| Unique peptides | 51385 | Unique peptides | 12417 |
| Total phosphopeptides | 797 | Total phosphopeptides | 12013 |
| Unique | 704 | Unique | 9963 |
| phosphopeptides | | phosphopeptides | |
| Unique S/T peptides | 649 | Unique S/T peptides | 9402 |
| Unique Y peptides | 67 | Unique Y peptides | 740 |
| Unique Proteins | 7716 | Unique Proteins | 3722 |
| Unique | 528 | Unique | 3108 |
| Phosphoproteins | | Phosphoproteins | |
| Mascot scr (no mod)* | 59 +/− 24 | Mascot scr (no mod)* | 49 +/− 21 |
| Mascot scr (phospho)* | 44 +/− 24 | Mascot scr (phospho)* | 49 +/− 21 |

*Average +/− SD

TABLE 8
Summary of clinical characteristics of sample cohort

| | | | | | | | Response | | | |
|---|---|---|---|---|---|---|---|---|---|---|
| | | Age | | | | | To | Patient | Progres- | |
| Kinomica | | at | Cytoge- | | FLT3 | FLT3 | Initial | Vital | sion.free.sur- | Re- |
| ID | Gender | Dx | netics | NPM1 | ITD | TKD | Tx | Status | vival.weeks | sponse.groups |
| K_AML_0001 | Male | 63 | Normal | Positive | Positive | Undetectable | CR | Deceased | 19.57143 | Negative |
| K_AML_0002 | Male | 53 | Abnormal | Positive | Positive | Undetectable | CR | Deceased | 17.42857 | Negative |
| K_AML_0003 | Female | 47 | Not | Positive | Positive | Undetectable | CR | Alive | 108.7143 | Positive |
| | | | assess- | | | | | | | |
| | | | able | | | | | | | |
| K_AML_0004 | Male | 79 | Normal | Undetectable | Positive | Undetectable | NR | Deceased | 0 | Negative |
| K_AML_0005 | Male | 64 | Normal | Positive | Positive | Undetectable | CR | Alive | 104.4286 | Positive |
| K_AML_0006 | Male | 48 | Normal | Positive | Positive | Undetectable | CR | Alive | 104.4286 | Positive |
| K_AML_0007 | Female | 55 | Normal | Positive | Undetectable | Positive | CR | Alive | 87 | Positive |
| K_AML_0008 | Female | 53 | Normal | Positive | Positive | Undetectable | CR | Deceased | 25.85714 | Negative |
| K_AML_0009 | Male | 77 | Abnormal | Positive | Positive | Undetectable | CR | Deceased | 47.71429 | Neutral |
| K_AML_0010 | Female | 65 | Abnormal | Undetectable | Positive | Undetectable | CR | Deceased | 17.14286 | Negative |
| K_AML_0011 | Female | 73 | Abnormal | Undetectable | Undetectable | Positive | CR | Deceased | 9.142857 | Negative |
| K_AML_0012 | Male | 74 | Abnormal | Undetectable | Positive | Undetectable | NR | Alive | 13.14286 | Negative |
| K_AML_0013 | Female | 45 | Normal | Positive | Positive | Undetectable | CR | Alive | 96 | Positive |
| K_AML_0014 | Male | 19 | Abnormal | Undetectable | Positive | Undetectable | NR | Alive | 88.42857 | Positive |
| K_AML_0015 | Female | 55 | Normal | Undetectable | Positive | Undetectable | CR | Alive | 87.14286 | Positive |
| K_AML_0016 | Female | 76 | Normal | Positive | Positive | Undetectable | CR | Alive | 87.28571 | Positive |
| K_AML_0017 | Male | 52 | Normal | Undetectable | Undetectable | Positive | NR | Alive | 39.14286 | Neutral |
| K_AML_0018 | Female | 48 | Abnormal | Positive | Positive | Undetectable | CR | Alive | 87.28571 | Positive |
| K_AML_0019 | Female | 31 | Normal | Positive | Undetectable | Positive | CR | Alive | 78.42857 | Neutral |
| K_AML_0020 | Male | 46 | Normal | Positive | Positive | Undetectable | CR | Alive | 87.28571 | Positive |
| K_AML_0021 | Female | 69 | Normal | Positive | Positive | Undetectable | NR | Deceased | 43.14286 | Neutral |
| K_AML_0022 | Female | 71 | Normal | Positive | Positive | Undetectable | CR | Alive | 78.57143 | Neutral |
| K_AML_0023 | Male | 69 | Normal | Positive | Positive | Undetectable | CR | Deceased | 4.428571 | Negative |
| K_AML_0024 | Female | 67 | Abnormal | Positive | Positive | Undetectable | CR | Alive | 69.71429 | Neutral |
| K_AML_0025 | Female | 78 | Abnormal | Undetectable | Undetectable | Positive | CR | Alive | 39.14286 | Neutral |
| K_AML_0026 | Male | 71 | Abnormal | Positive | Positive | Undetectable | CR | Alive | 69.71429 | Neutral |
| K_AML_0027 | Female | 61 | Abnormal | Undetectable | Undetectable | Positive | CR | Deceased | 39.14286 | Neutral |
| K_AML_0028 | Male | 61 | Normal | Positive | Positive | Positive | CR | Alive | 69.71429 | Neutral |
| K_AML_0029 | Female | 60 | Normal | Positive | Positive | Undetectable | CR | Alive | 61 | Neutral |
| K_AML_0030 | Female | 56 | Failure | Positive | Positive | Undetectable | CR | Deceased | 21.71429 | Negative |
| K_AML_0031 | Female | 77 | Abnormal | Undetectable | Positive | Undetectable | CR | Alive | 56.57143 | Neutral |
| K_AML_0032 | Female | 72 | Abnormal | Undetectable | Positive | Undetectable | NR | Deceased | 7.714286 | Negative |
| K_AML_0033 | Male | 61 | Normal | Positive | Positive | Undetectable | CR | Alive | 56.71429 | Neutral |
| K_AML_0034 | Female | 77 | Normal | Positive | Undetectable | Positive | CR | Alive | 48 | Neutral |
| K_AML_0035 | Female | 38 | Abnormal | Inconclusive | Positive | Undetectable | NR | Deceased | 39.28571 | Neutral |
| K_AML_0036 | Male | 31 | not done | Positive | Positive | Undetectable | NR | Alive | 34.85714 | Negative |
| K_AML_0037 | Female | 65 | Normal | Positive | Positive | Undetectable | CR | Alive | 39.14286 | Neutral |
| K_AML_0038 | Male | 70 | Normal | Positive | Positive | Undetectable | CR | Alive | 34.71429 | Negative |
| K_AML_0039 | Female | 70 | Normal | Positive | Positive | Undetectable | CR | Alive | 52.14286 | Neutral |
| K_AML_0040 | Male | 55 | Normal | Positive | Positive | | CR | Alive | 147.8571 | Positive |
| K_AML_0041 | Male | 37 | Abnormal | Undetectable | Positive | Undetectable | CR | Alive | 152.1429 | Positive |
| K_AML_0042 | Female | 28 | Normal | Positive | Positive | Undetectable | CR | Alive | 152.1429 | Positive |
| K_AML_0043 | Male | 59 | Normal | Positive | Positive | Undetectable | CR | Alive | 78.42857 | Neutral |
| K_AML_0044 | Male | 52 | Normal | Positive | Undetectable | Positive | CR | Alive | 126.2857 | Positive |
| K_AML_0045 | Male | 55 | Abnormal | Undetectable | Positive | Undetectable | CR | Deceased | 13.14286 | Negative |
| K_AML_0046 | Female | 41 | Normal | Positive | Positive | Undetectable | CR | Alive | 143.7143 | Positive |
| K_AML_0047 | Female | 47 | Normal | Positive | Positive | Positive | CR | Alive | 30.57143 | Negative |
| K_AML_0048 | Female | 68 | Normal | Positive | Positive | Undetectable | CR | Alive | 60.85714 | Neutral |
| K_AML_0049 | Male | 74 | Normal | Undetectable | Positive | | CR | Deceased | 43.42857 | Neutral |
| K_AML_0050 | Male | 80 | Not | Positive | Positive | | CR | Deceased | 21.42857 | Negative |
| | | | assess- | | | | | | | |
| | | | able | | | | | | | |
| K_AML_0051 | Female | 59 | Not | Positive | Positive | Undetectable | CR | Alive | 126.2857 | Positive |
| | | | assess- | | | | | | | |
| | | | able | | | | | | | |
| K_AML_0052 | Female | 64 | Normal | Positive | Undetectable | Positive | CR | Alive | 108.8571 | Positive |
| K_AML_0053 | Female | 31 | Abnormal | Undetectable | Undetectable | Positive | CR | Alive | 34.57143 | Negative |
| K_AML_0054 | Female | 53 | Normal | Positive | Positive | Undetectable | CR | Deceased | 21.85714 | Negative |
| K_AML_0055 | Male | 59 | Normal | Positive | Undetectable | Positive | CR | Alive | 478.2857 | Positive |
| K_AML_0056 | Female | 58 | Normal | Positive | Positive | Undetectable | CR | Deceased | 343.7143 | Positive |
| K_AML_0057 | Female | 73 | Normal | Positive | Positive | Undetectable | CR | Alive | 48 | Neutral |
| K_AML_0058 | Male | 59 | Normal | Positive | Positive | Undetectable | CR | Deceased | 91.28571 | Positive |
| K_AML_0059 | Female | 37 | Normal | Positive | Positive | Undetectable | NR | Deceased | 43.42857 | Neutral |
| K_AML_0060 | Male | 42 | Normal | Positive | Positive | Undetectable | CR | Alive | 513.1429 | Positive |

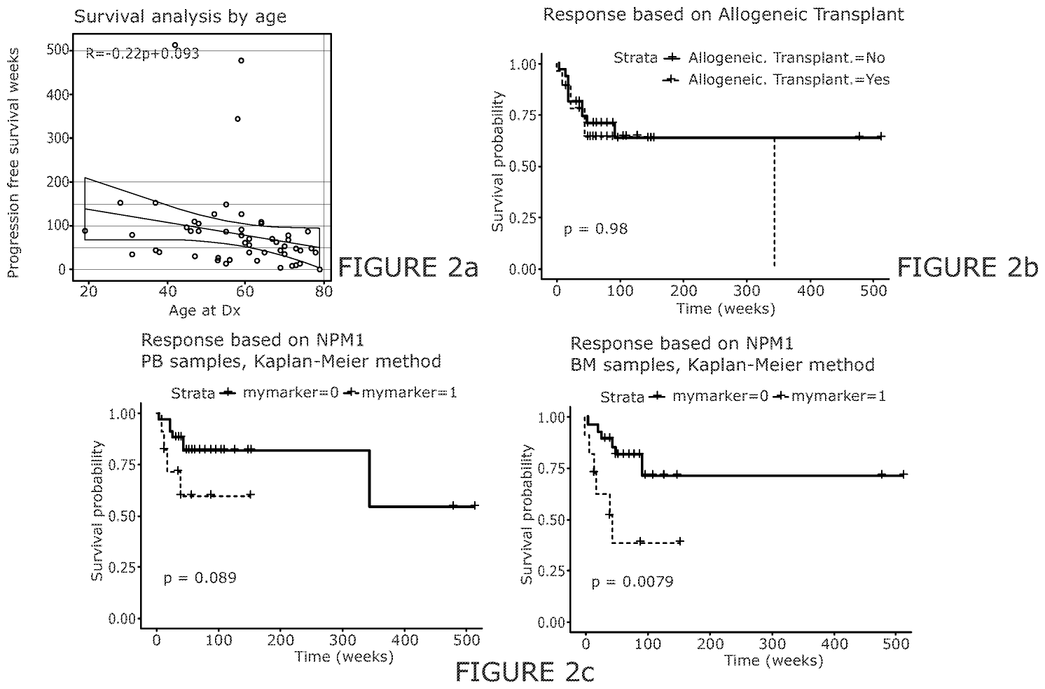

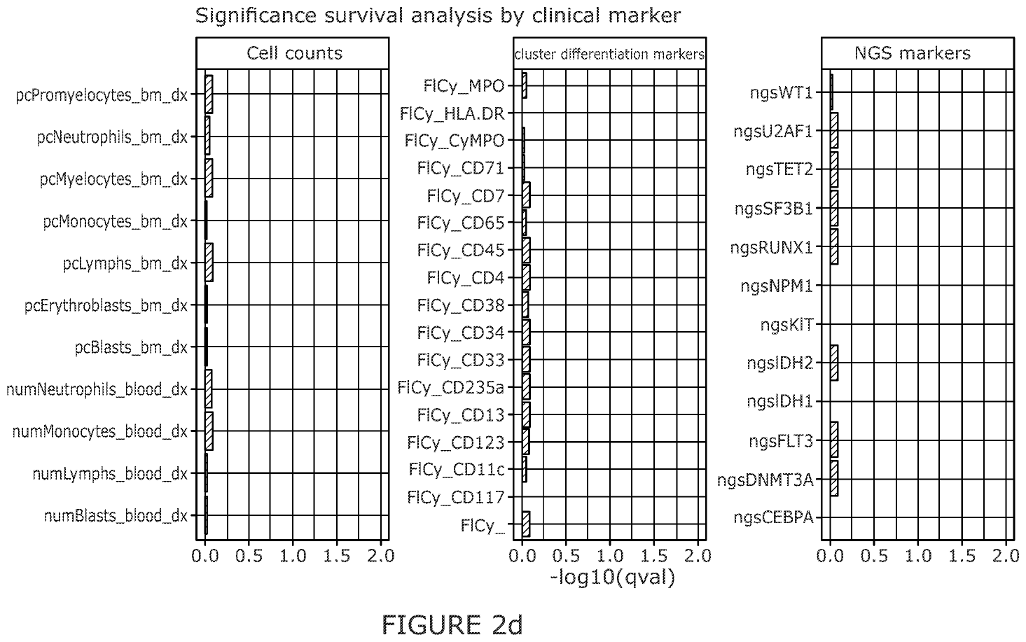

Consistent with previous studies, older patients tended to have less favourable treatment outcome than younger individuals but this association was not statistically significant (FIG. 2a). Allogeneic BM transplantation had not an effect on survival probability in this patient population (FIG. 2b), which may be due to the short time of assessment. Patients with mutated NPM1 survived longer than non NPM1 mutant cases (FIG. 2c). Other genetic mutations or expression of CD markers had no impact on patient survival (FIG. 2d).

### Example 2—Phosphoproteomic Signatures Associated with Responses to Midostaurin Plus Chemotherapy

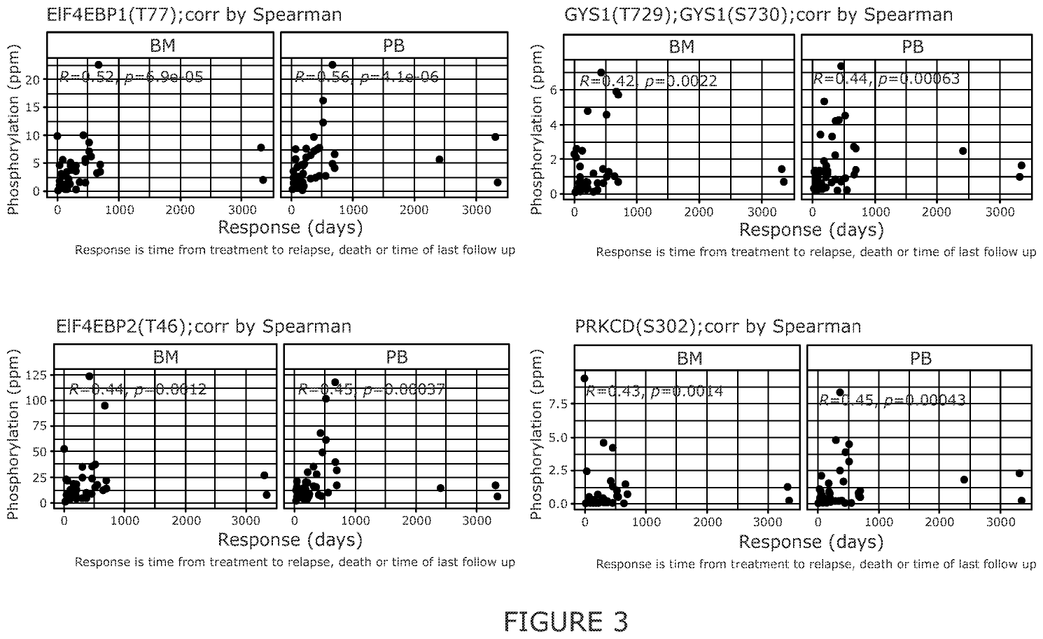

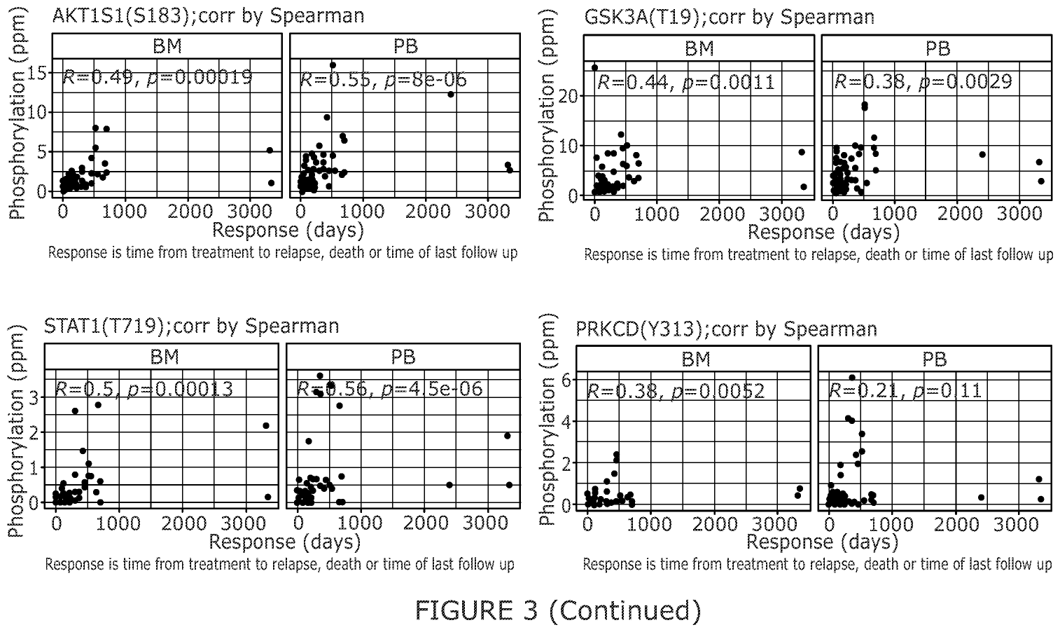

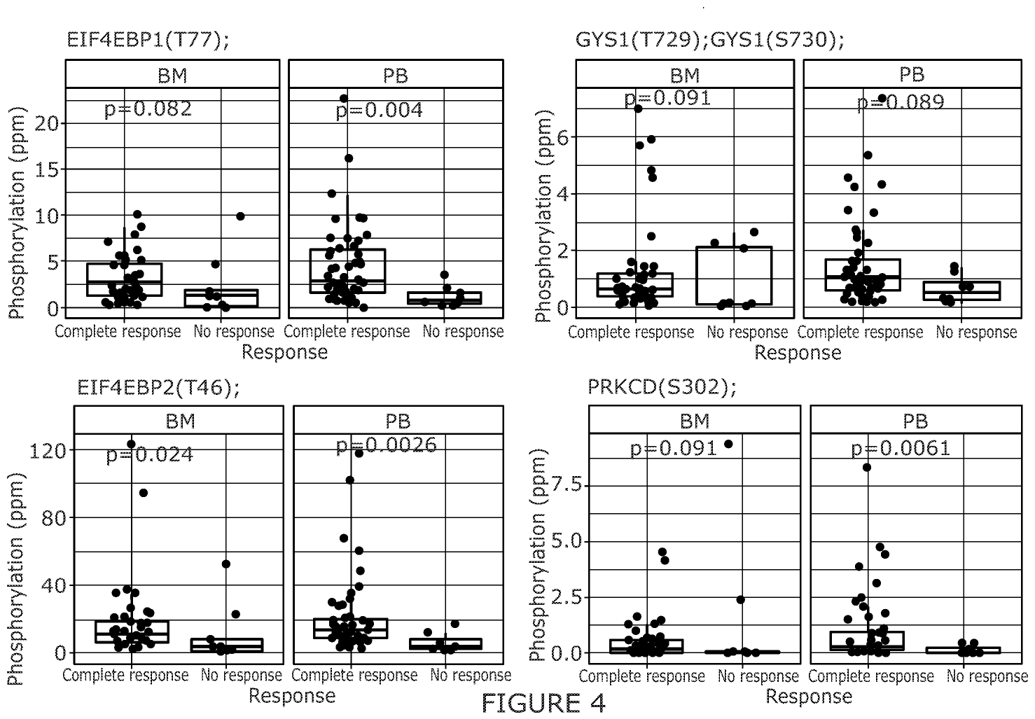

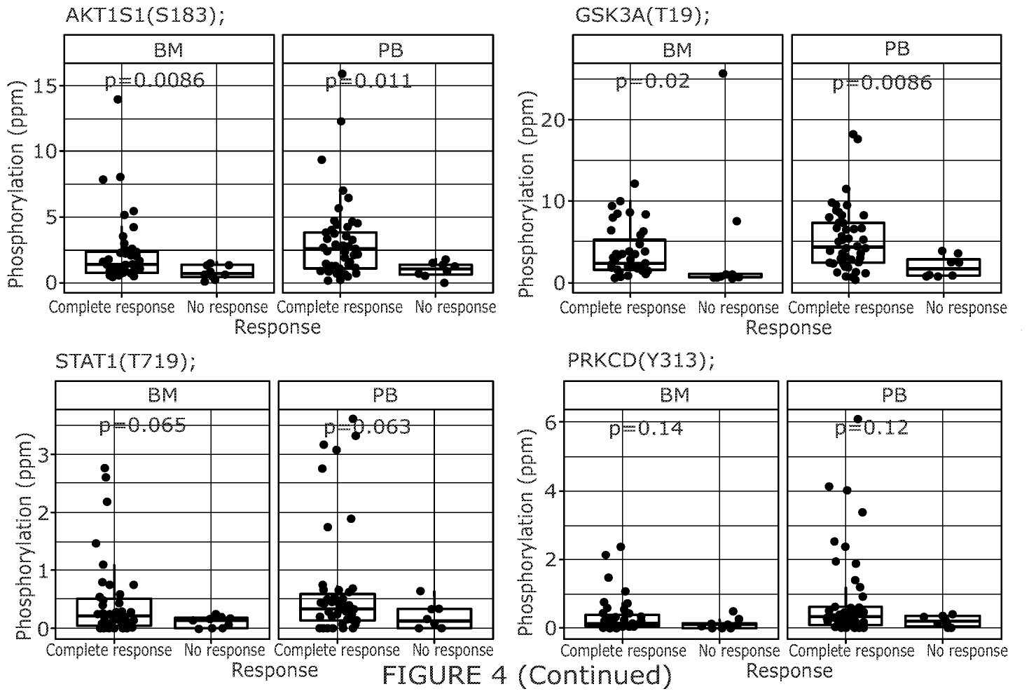

We first assessed whether phosphoproteomic signatures, previously identified to be associated with ex vivo responses to midostaurin, were also correlated with clinical responses to this drug. To address this, we targeted the quantification of these phosphorylation sites which we then associated to progression free survival. The intensities of the phosphopeptide of interest were first normalized to the signals of high abundant phosphopeptides within samples. The list of phosphopeptides used for normalization is shown in Table 7. This analysis showed several phosphorylation sites on STAT, MTOR and PKC delta (gene name PRKCD) pathway members (including STAT1, 4EBP1, 4EBP2, AKT1S1, and GSK3A) to be significantly correlated with responses (FIG. 3 corr plots and 4 box plots). These are interesting results because PKC is a midostaurin target and MTOR and STATs are downstream effectors of several receptor tyrosine kinases, including FLT3.

We then conducted differential survival analysis using the Kepler Meier method and log rank statistics. To do this, patients were stratified based on the expression of the individual makers into those that showed high or low phosphorylation using the median expression across patients as threshold. These analysis was carried out separately for samples obtained from BM or peripheral blood (PB).

In addition to assessing the association between single phosphosites and patient survival, we also considered patient survival as a function of phosphopeptide ratios. This analysis was undertaken as a way of normalizing these measurements as would be done to facilitate the translation of these markers into a clinical assay. We thus considered the intensity of a given phosphopeptide found to be increased in patient samples that responded well to therapy relative to (divided by) the intensities of phosphopeptides found to be decreased in the same samples. Finally, we also trained machine learning models using the set of phosphopeptide biomarkers mentioned above plus new set of phosphosites found to be differentially expressed in patient samples showing extreme response values.

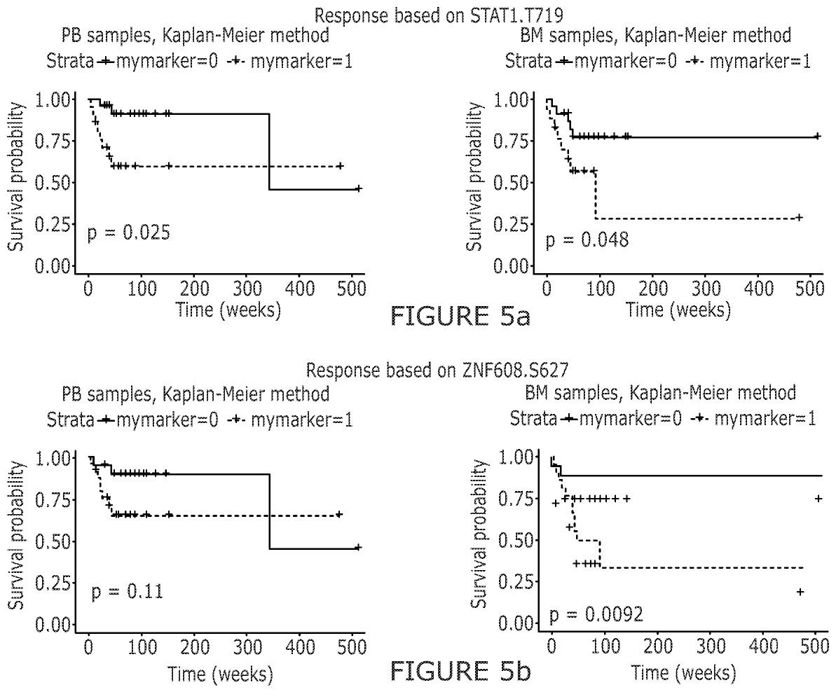

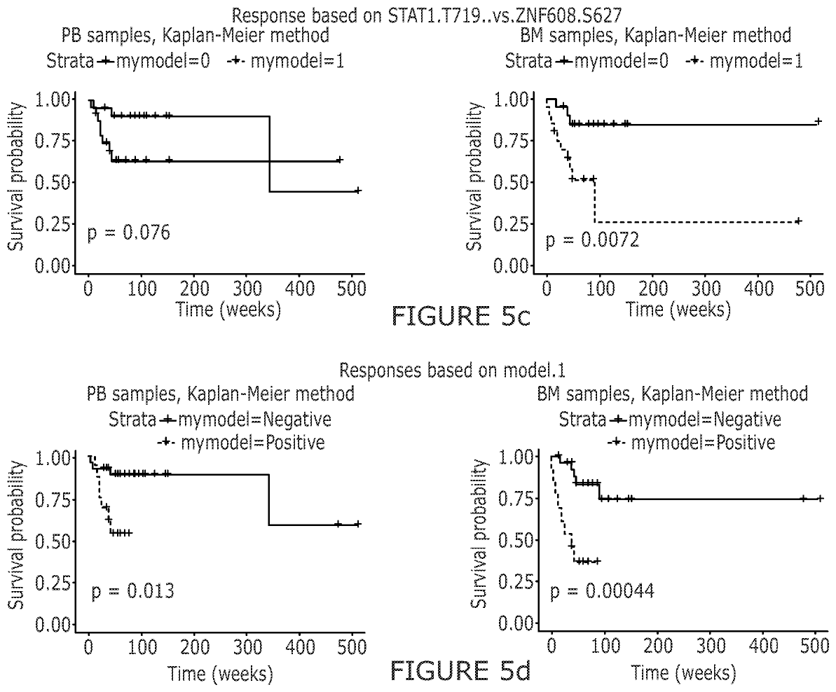

FIGS. 5a and 5b show examples of survival curves stratified by the phosphorylation of single phosphosite markers. These data illustrate that patients with high phosphorylation of STAT1 at T719 have greater probability of survival than those with low phosphorylation at this site, whereas patients with high phosphorylation of ZNF608 at S627 is associated with low survival. We then determined a response index defined as the ratio between the phosphorylation of STAT1 at T719 and ZNF608 at S627. Patients with high response index had a longer survival probability than other patients (Figure Sc). Finally, we trained machine learning models using the random forest algorithm using as predictors the different phosphorylation sites found in this study to be associates with responses. Labels for model generation were obtained by classifying patients as “negative” if they have a progression free survival<12 months or “positive” if their progression free survival was greater than this cut-off value. We applied a 10-fold cross validation strategy for model generation and testing. The results of patient classification based on one of the models, shown in FIG. 5d, revealed that this machine learning (ML) model could discriminate between positive (i.e., likely responders) and negative (i.e., likely resistant) patients with very good discriminatory power from samples obtained from both BM (p=0.013)) and PB (p=0.00044). Other ML models were trained and evaluated. Details of these are given in Table 10.

We evaluated other markers of responses by themselves or as ratios and other ML models (Tables 9 and 10). This revealed that the response index consisting of AKT1S1 S183 divided by ZNF608 S627 was particularly effective in patient stratification (p=1.55E-04 for BM samples and p=3.82E-02 for PB samples). ML model 3 (based on phosphorylation sites only as predictors) performed better from PB samples whereas ML model 6 (phosphosites plus genetic mutations) was the best performer in BM samples.

TABLE 9
Phosphopeptides used for normalisation
Phosphopeptide loading controls

GSK3A(S282); GSK3B(S219);
GSK3A(Y279); GSK3B(Y216);
NELFA(S363);
UNK(S374); UNK(S385);
UNK(S385);
CIC(S902);
RABEP1(S410);
ANKLE2(T665);
PI4KB(S428);
TFPT(S180);
POLR2A(Y1909);

TABLE 10
Phosphosite markers used as input (predictors) of ML models

| ML models | Phosphosite markers used for prediction |
|---|---|
| Model1, corr | MYC.T58 . . . MYCN.T58 . . . ; MYC.T58 . . . ; H2AFY.S316 . . . ; KDM2B.S1029 . . . ; KMT2D.S3620 . . . ; EIF4EBP1.T77 . . . ; |
| | EIF4EBP2.T46 . . . ; AKT1S1.S183 . . . ; STAT1.T719 . . . ; GYS1.T729 . . . GYS1.S730 . . . ; PRKCD.S302 .; GSK3A.T19 . . . ; |
| | PRKCD.Y313 . . . |
| Model 2, incr | BRAF.S363 . . . ; JAK3.S15 . . . JAK3.S17 . . . ; ATM.S2996 . . . ; VIM.T327 . . . ; PSD3.S1009 . . . ; EPN1.S447 . . . ; |
| relapse and ext | CASP9.S310 . . . ; MARCKS.T120 . . . ; CDK13.S1054 . . . ; DEPTOR.S244 . . . ; ZNF608.S627 . . . ; MINK1.S772 . . . ; |
| pheno | CAMK1D.S179 . . . |
| Model 3, all | MYC.T58 . . . MYCN.T58 . . . ; MYC.T58 . . . ; H2AFY.S316 . . . ; KDM2B.S1029 . . . ; KMT2D.S3620 . . . ; EIF4EBP1.T77 . . . ; |
| phospho | EIF4EBP2.T46 . . . ; AKT1S1.S183 . . . ; STAT1.T719 . . . ; GYS1.T729 . . . GYS1.S730 . . . ; PRKCD.S302 . . . ; GSK3A.T19 . . . ; |
| | PRKCD.Y313 . . . ; BRAF.S363 . . . ; JAK3.S15 . . . JAK3.S17 . . . ; ATM.S2996 . . . ; VIM.T327 . . . ; PSD3.S1009 . . . ; |
| | EPN1.S447 . . . ; CASP9.S310 . . . ; MARCKS.T120 . . . ; CDK13.S1054 . . . ; DEPTOR.S244 . . . ; ZNF608.S627 . . . ; |
| | MINK1.S772 . . . ; CAMK1D.S179 . . . |
| Model 4, top | ZNF608.S627 . . . ; CDK13.S1054 . . . ; CAMK1D.S179 . . . ; STAT1.T719 . . . ; PSD3.S1009 . . . ; GSK3A.T19 . . . ; |
| markers | KDM2B.S1029 . . . ; MINK1.S772 . . . ; DEPTOR.S244 . . . ; MARCKS.T120 . . . ; MYC.T58 . . . ; GYS1.T729 . . . GYS1.S730 . . . ; |
| | MYC.T58 . . . MYCN.T58 . . . ; JAK3.S15 . . . JAK3.S17 . . . ; KMT2D.S3620 . . . ; AKT1S1.S183 . . . ; PRKCD.Y313 . . . |
| Model 5, genes | FLT3.ITD.levels.0.1; FLT3.TKD.0.1; NPM1 |
| only | |
| Model 6, all | MYC.T58 . . . MYCN.T58 . . . ; MYC.T58 . . . ; H2AFY.S316 . . . ; KDM2B.S1029 . . . ; KMT2D.S3620 . . . ; EIF4EBP1.T77 . . . ; |
| phospho plus | EIF4EBP2.T46 . . . ; AKT1S1.S183 . . . ; STAT1.T719 . . . ; GYS1.T729 . . . GYS1.S730 . . . ; PRKCD.S302 . . . ; GSK3A.T19 . . . ; |
| genes | PRKCD.Y313 . . . ; BRAF.S363 . . . ; JAK3.S15 . . . JAK3.S17 . . . ; ATM.S2996 . . . ; VIM.T327 . . . ; PSD3.S1009 . . . ; |
| | EPN1.S447 . . . ; CASP9.S310 . . . ; MARCKS.T120 . . . ; CDK13.S1054 . . . ; DEPTOR.S244 . . . ; ZNF608.S627 . . . ; |
| | MINK1.S772 . . . ; CAMK1D.S179 . . . ; FLT3.ITD.levels.0.1; FLT3.TKD.0.1; NPM1 |
| Model 7. | NPM1; MYC.T58 . . . MYCN.T58 . . . ; MYC.T58 . . . ; H2AFY.S316 . . . ; KDM2B.S1029 . . . ; KMT2D.S3620 . . . ; |
| phospho plus | EIF4EBP1.T77 . . . ; EIF4EBP2.T46 . . . ; AKT1S1.S183 . . . ; STAT1.T719 . . . ; GYS1.T729 . . . GYS1.S730 . . . ; |
| NPM1 | PRKCD.S302 . . . ; GSK3A.T19 . . . ; PRKCD.Y313 . . . ; BRAF.S363 . . . ; JAK3.S15 . . . JAK3.S17 . . . ; ATM.S2996 . . . ; |
| | VIM.T327 . . . ; PSD3.S1009 . . . ; EPN1.S447 . . . ; CASP9.S310 . . . ; MARCKS.T120 . . . ; CDK13.S1054 . . . ; |
| | DEPTOR.S244 . . . ; ZNF608.S627 . . . ; MINK1.S772 . . . ; CAMK1D.S179 . . . |

TABLE 11
Survival analysis bone marrow samples

| signature | pvalue | qvalue | av.hi | av.low | n.hi | n.low |
|---|---|---|---|---|---|---|
| Single markers | | | | | | |
| NPM1 | 7.95E−03 | 0.038955 | 380.6 | 213.3 | 28 | 11 |
| ZNF608 S627 | 9.18E−03 | 0.040736 | 200.4 | 457.1 | 22 | 18 |
| PSD3 S1009 | 5.91E−01 | 0.69935 | 258.7 | 362.0 | 17 | 23 |
| STAT5A S780 | 2.09E−02 | 0.087288 | 255.6 | 475.7 | 27 | 13 |
| STAT1 T719 | 4.82E−02 | 0.139542 | 401.9 | 179.6 | 23 | 17 |
| PRKCD S302 | 3.07E−02 | 0.107176 | 447.2 | 222.5 | 16 | 24 |
| MINK1 S772 | 4.93E−02 | 0.139542 | 244.5 | 435.1 | 21 | 19 |
| KMT2D S3620 | 5.11E−02 | 0.139542 | 303.7 | 428.8 | 29 | 11 |
| EIF4EBP2 T46 | 6.65E−01 | 0.737734 | 361.4 | 260.8 | 21 | 19 |
| DEPTOR S244 | 4.86E−01 | 0.605368 | 331.4 | 351.5 | 25 | 15 |
| GYS1 T729 | 7.54E−02 | 0.183934 | 436.8 | 234.7 | 14 | 26 |
| GYS1 S730 | | | | | | |
| EIF4EBP1 T77 | 8.29E−02 | 0.183934 | 402.7 | 190.3 | 19 | 21 |
| ATM S2996 | 4.68E−01 | 0.605368 | 346.3 | 323.7 | 22 | 18 |
| CDK13 S1054 | 3.12E−01 | 0.4615 | 324.5 | 350.9 | 22 | 18 |
| EPN1 S447 | 1.87E−01 | 0.331925 | 391.0 | 199.6 | 17 | 23 |
| AKT1S1 S183 | 1.28E−01 | 0.245622 | 392.6 | 250.8 | 22 | 18 |
| FLT3 TKD 0 1 | 1.52E−01 | 0.284 | 453.9 | 288.2 | 9 | 31 |
| FLT3 ITD | 9.46E−01 | 0.97342 | 393.5 | 300.1 | 9 | 11 |
| levels 0 1 | | | | | | |
| CAMK1D S179 | 5.50E−01 | 0.661864 | 334.9 | 345.9 | 24 | 16 |
| MYC TSS | 1.94E−01 | 0.335951 | 271.3 | 418.8 | 30 | 10 |
| MYCN T58 | | | | | | |
| PRKCD Y313 | 2.52E−01 | 0.388957 | 390.9 | 267.3 | 17 | 23 |
| KDM2B S1029 | 9.74E−01 | 0.987914 | 315.1 | 346.8 | 27 | 13 |
| MARCKS T120 | 2.35E−01 | 0.370778 | 366.7 | 319.9 | 19 | 21 |
| BRAF S363 | 2.83E−01 | 0.427511 | 345.0 | 306.3 | 23 | 17 |
| CASP9 S310 | 3.25E−01 | 0.470918 | 396.0 | 266.4 | 18 | 22 |
| VIM T327 | 5.03E−01 | 0.615741 | 392.4 | 273.8 | 17 | 23 |
| JAK3 S15 | 3.55E−01 | 0.503961 | 305.6 | 389.3 | 26 | 14 |
| JAK3 S17 | | | | | | |
| H2AFY S316 | 6.46E−01 | 0.728032 | 297.7 | 367.9 | 30 | 10 |
| GSK3A T19 | 8.92E−01 | 0.943882 | 347.8 | 291.9 | 16 | 24 |
| PRKCD S506 | 4.78E−01 | 0.605368 | 298.5 | 376.0 | 22 | 18 |
| Allogeneic | 9.04E−01 | 0.943882 | 311.1 | 342.9 | 21 | 19 |
| Transplant 0 1 | | | | | | |
| MYC T58 | 9.89E−01 | 0.989 | 296.0 | 340.4 | 26 | 14 |
| ML models | | | | | | |
| model 6 | 4.02E−07 | 2.85E−05 | 403.7 | 99.8 | 29 | 11 |
| model 3 | 2.83E−06 | 5.02E−05 | 403.2 | 116.1 | 28 | 12 |
| model 7 | 2.83E−06 | 5.02E−05 | 403.2 | 116.1 | 28 | 12 |
| model 4 | 2.83E−06 | 5.02E−05 | 403.2 | 116.1 | 28 | 12 |
| model 2 | 7.68E−06 | 0.000109 | 415.3 | 140.5 | 26 | 14 |
| model 1 | 4.44E−04 | 0.004503 | 396.0 | 201.9 | 27 | 13 |
| model 8 | 7.82E−02 | 0.183934 | 351.8 | 287.0 | 22 | 18 |
| model 5 | 4.40E−01 | 0.589434 | 320.8 | 513.1 | 38 | 2 |
| Phosphopeptide | | | | | | |
| ratios | | | | | | |
| AKT1S1 S183 vs | 1.55E−04 | 0.001834 | 486.8 | 142.9 | 19 | 21 |
| ZNF608 S627 | | | | | | |
| PRKCD Y313 vs | 1.14E−03 | 0.010118 | 513.1 | 200.1 | 14 | 26 |
| ZNF608 S627 | | | | | | |
| STAT1 T719 vs | 4.26E−03 | 0.033607 | 439.0 | 167.1 | 20 | 20 |
| STAT5A S780 | | | | | | |
| PRKCD S302 vs | 6.09E−03 | 0.038955 | 455.1 | 60.5 | 18 | 22 |
| STAT5A S780 | | | | | | |
| GSK3A T19 vs | 6.96E−03 | 0.038955 | 478.9 | 208.1 | 15 | 25 |
| ZNF608 S627 | | | | | | |
| EIF4EBP2 T46 vs | 6.96E−03 | 0.038955 | 478.9 | 208.1 | 15 | 25 |
| ZNF608 S627 | | | | | | |
| STAT1 T719 vs | 7.17E−03 | 0.038955 | 438.5 | 166.3 | 20 | 20 |
| ZNF608 S627 | | | | | | |
| PRKCD S302 vs | 8.23E−03 | 0.038955 | 478.9 | 176.1 | 15 | 25 |
| ZNF608 S627 | | | | | | |
| STAT1 T719 vs | 3.93E−02 | 0.121317 | 384.6 | 287.6 | 19 | 21 |
| CDK13 S1054 | | | | | | |
| PRKCD Y313 vs | 6.80E−02 | 0.178815 | 384.6 | 295.2 | 19 | 21 |
| DEPTOR S244 | | | | | | |
| PRKCD S302 vs | 7.54E−02 | 0.183934 | 436.8 | 234.7 | 14 | 26 |
| DEPTOR S244 | | | | | | |
| GSK3A T19 vs | 2.68E−02 | 0.105711 | 473.7 | 242.7 | 13 | 27 |
| STATSA S780 | | | | | | |
| AKT1S1 S183 vs | 3.08E−02 | 0.107176 | 432.0 | 212.5 | 18 | 22 |
| STATSA S780 | | | | | | |
| PRKCD Y313 vs | 3.17E−02 | 0.107176 | 466.2 | 250.4 | 12 | 28 |
| STAT5A S780 | | | | | | |
| STAT1 T719 vs | 3.78E−02 | 0.121317 | 409.7 | 183.0 | 20 | 20 |
| MYC T58 | | | | | | |
| EIF4EBP2 T46 vs | 8.19E−02 | 0.183934 | 376.1 | 301.0 | 18 | 22 |
| CDK13 S1054 | | | | | | |
| AKT1S1 5183 vs | 9.61E−02 | 0.19738 | 368.0 | 301.0 | 18 | 22 |
| CDK13 S1054 | | | | | | |
| EIF4EBP2 T46 vs | 2.25E−01 | 0.363068 | 372.1 | 310.7 | 17 | 23 |
| DEPTOR S244 | | | | | | |
| PRKCD S302 vs | 9.11E−02 | 0.196003 | 395.1 | 301.3 | 16 | 24 |
| CDK13 S1054 | | | | | | |
| GSK3A T19 vs | 9.73E−02 | 0.19738 | 377.8 | 279.6 | 22 | 18 |
| DEPTOR S244 | | | | | | |
| PRKCD Y313 vs | 1.02E−01 | 0.201167 | 376.9 | 300.2 | 18 | 22 |
| CDK13 S1054 | | | | | | |
| STAT1 T719 vs | 6.37E−01 | 0.728032 | 343.1 | 337.9 | 20 | 20 |
| DEPTOR S244 | | | | | | |
| PRKCD S302 vs | 1.58E−01 | 0.287641 | 405.5 | 199.6 | 15 | 25 |
| MYC T58 | | | | | | |
| Ratio Sum PKC | 2.10E−01 | 0.355 | 414.9 | 248.0 | 10 | 30 |
| GSK3 STAT1 | | | | | | |
| 4EBP2 AKT1S1 | | | | | | |
| vs CDK13 | | | | | | |
| ZNF608 0 | | | | | | |
| to 1 | | | | | | |
| EIF4EBP2 T46 vs | 2.22E−01 | 0.363068 | 394.8 | 243.1 | 17 | 23 |
| STAT5A S780 | | | | | | |
| EIF4EBP2 T46 vs | 4.06E−01 | 0.554346 | 378.3 | 253.7 | 15 | 25 |
| MYC T58 | | | | | | |
| AKT1S1 S183 vs | 8.17E−01 | 0.892415 | 325.9 | 339.3 | 19 | 21 |
| DEPTOR S244 | | | | | | |
| GSK3A T19 vs | 3.62E−01 | 0.503961 | 351.5 | 327.8 | 15 | 25 |
| CDK13 S1054 | | | | | | |
| AKT1S1 S183 vs | 6.07E−01 | 0.706508 | 356.6 | 263.8 | 16 | 24 |
| MYC T58 | | | | | | |
| GSK3A T19 vs | 4.77E−01 | 0.605368 | 380.7 | 280.7 | 11 | 29 |
| MYC T58 | | | | | | |
| PRKCD Y313 vs | 8.73E−01 | 0.939136 | 344.7 | 292.8 | 15 | 25 |
| MYC T58 | | | | | | |

TABLE 12
Survival analysis peripheral blood samples

| signature | pvalue | qvalue | av.hi | av.low | n.hi | n.low |
|---|---|---|---|---|---|---|
| Single markers | | | | | | |
| NPM1 | 8.88E−02 | 0.279694 | 378.1 | 314.6 | 34 | 11 |
| ZNF608 S627 | 1.07E−01 | 0.302913 | 342.8 | 387.1 | 25 | 20 |
| PSD3 S1009 | 2.04E−02 | 0.15649 | 228.4 | 464.7 | 23 | 22 |
| STAT5A S780 | 3.92E−01 | 0.604605 | 352.6 | 355.8 | 29 | 16 |
| STAT1 T719 | 2.55E−02 | 0.15649 | 393.0 | 313.2 | 24 | 21 |
| PRKCD S302 | 8.22E−01 | 0.918613 | 336.2 | 395.0 | 23 | 22 |
| MINK1 S772 | 8.41E−01 | 0.918613 | 411.6 | 331.5 | 20 | 25 |
| KMT2D S3620 | 9.36E−01 | 0.957675 | 387.9 | 351.8 | 34 | 11 |
| EIF4EBP2 T46 | 7.04E−02 | 0.269601 | 462.4 | 292.0 | 20 | 25 |
| DEPTOR S244 | 7.32E−02 | 0.269601 | 299.5 | 477.1 | 31 | 14 |
| GYS1 T729 | 1.67E−01 | 0.408774 | 381.4 | 357.9 | 19 | 26 |
| GYS1 S730 | | | | | | |
| EIF4EBP1 T77 | 7.16E−01 | 0.847444 | 353.2 | 382.5 | 17 | 28 |
| ATM S2996 | 9.06E−02 | 0.279694 | 433.8 | 235.7 | 27 | 18 |
| CDK13 S1054 | 9.71E−02 | 0.287242 | 335.7 | 390.1 | 18 | 27 |
| EPN1 S447 | 1.11E−01 | 0.302913 | 376.1 | 335.5 | 25 | 20 |
| AKT1S1 S183 | 2.40E−01 | 0.449206 | 364.6 | 341.9 | 27 | 18 |
| FLT3 TKD 0 1 | 6.22E−01 | 0.77415 | 413.5 | 329.1 | 11 | 34 |
| FLT3 ITD | 1.54E−01 | 0.390419 | 513.1 | 396.5 | 10 | 13 |
| levels 0 1 | | | | | | |
| CAMK1D S179 | 1.87E−01 | 0.423257 | 356.6 | 403.7 | 28 | 17 |
| MYC T58 | 2.27E−01 | 0.447113 | 332.3 | 467.3 | 34 | 11 |
| MYCN TS8 | | | | | | |
| PRKCD Y313 | 1.97E−01 | 0.423257 | 308.5 | 436.0 | 25 | 20 |
| KDM2B S1029 | 2.14E−01 | 0.435388 | 317.4 | 464.4 | 35 | 10 |
| MARCKS T120 | 9.44E−01 | 0.957675 | 358.2 | 394.0 | 23 | 22 |
| BRAF S363 | 7.28E−01 | 0.847444 | 394.9 | 331.0 | 24 | 21 |
| CASP9 S310 | 5.25E−01 | 0.703662 | 288.5 | 364.7 | 24 | 21 |
| VIM T327 | 3.38E−01 | 0.557492 | 365.9 | 340.9 | 27 | 18 |
| JAK3 S15 | 9.18E−01 | 0.957675 | 382.3 | 346.0 | 25 | 20 |
| JAK3 S17 | | | | | | |
| H2AFY S316 | 3.75E−01 | 0.591285 | 374.3 | 363.9 | 28 | 17 |
| GSK3A T19 | 4.04E−01 | 0.609775 | 366.1 | 355.4 | 20 | 25 |
| PRKCD S506 | 4.60E−01 | 0.654898 | 357.8 | 364.7 | 25 | 20 |
| Allogeneic | 4.63E−01 | 0.654898 | 264.1 | 405.6 | 25 | 20 |
| Transplant 0 1 | | | | | | |
| MYC T58 | 8.41E−01 | 0.918613 | 357.0 | 390.3 | 33 | 12 |
| ML models | | | | | | |
| model 6 | 7.25E−03 | 0.085804 | 399.9 | 252.6 | 31 | 14 |
| model 3 | 1.47E−04 | 0.005219 | 414.7 | 180.4 | 32 | 13 |
| model 7 | 1.47E−04 | 0.005219 | 414.7 | 180.4 | 32 | 13 |
| model 4 | 4.64E−04 | 0.010981 | 424.6 | 249.6 | 28 | 17 |
| model 2 | 3.16E−03 | 0.044858 | 410.8 | 250.9 | 29 | 16 |
| model 1 | 1.26E−02 | 0.127678 | 407.9 | 291.2 | 28 | 17 |
| model 8 | 1.12E−03 | 0.019845 | 411.1 | 264.5 | 30 | 15 |
| model 5 | 5.00E−01 | 0.682956 | 351.1 | 513.1 | 43 | 2 |
| Phosphopeptide | | | | | | |
| ratios | | | | | | |
| AKT1S1 S183 vs | 3.82E−02 | 0.208451 | 391.8 | 318.8 | 23 | 22 |
| ZNF608 S627 | | | | | | |
| PRKCD Y313 vs | 2.15E−01 | 0.435388 | 372.6 | 344.9 | 22 | 23 |
| ZNF608 S627 | | | | | | |
| STAT1 T719 vs | 2.35E−01 | 0.449206 | 425.1 | 249.5 | 23 | 22 |
| STAT5A S780 | | | | | | |
| PRKCD S302 vs | 6.10E−01 | 0.773738 | 371.9 | 378.5 | 21 | 24 |
| STAT5A S780 | | | | | | |
| GSK3A T19 vs | 5.18E−02 | 0.229787 | 391.0 | 325.8 | 22 | 23 |
| ZNF608 S627 | | | | | | |
| EIF4EBP2 T46 vs | 5.18E−02 | 0.229787 | 391.0 | 325.8 | 22 | 23 |
| ZNF608 S627 | | | | | | |
| STAT1 T719 vs | 7.59E−02 | 0.269601 | 389.2 | 334.7 | 21 | 24 |
| ZNF608 S627 | | | | | | |
| PRKCD S302 vs | 3.10E−01 | 0.524835 | 368.9 | 356.9 | 21 | 24 |
| ZNF608 S627 | | | | | | |
| STAT1 T719 vs | 1.73E−02 | 0.153555 | 417.5 | 318.8 | 23 | 22 |
| CDK13 S1054 | | | | | | |
| PRKCD Y313 vs | 2.57E−02 | 0.15649 | 415.2 | 330.3 | 22 | 23 |
| DEPTOR S244 | | | | | | |
| PRKCD S302 vs | 2.64E−02 | 0.15649 | 407.5 | 317.3 | 20 | 25 |
| DEPTOR S244 | | | | | | |
| GSK3A T19 vs | 8.04E−01 | 0.918613 | 405.1 | 327.6 | 14 | 31 |
| STAT5A S780 | | | | | | |
| AKT1S1 S183 vs | 1.91E−01 | 0.423257 | 373.6 | 342.6 | 23 | 22 |
| STATSA S780 | | | | | | |
| PRKCD Y313 vs | 6.05E−01 | 0.773738 | 405.3 | 264.7 | 19 | 26 |
| STAT5A S780 | | | | | | |
| STAT1 T719 vs | 5.61E−01 | 0.737549 | 354.9 | 370.8 | 23 | 22 |
| MYC T58 | | | | | | |
| EIF4EBP2 T46 vs | 4.81E−02 | 0.229787 | 402.0 | 336.0 | 24 | 21 |
| CDK13 S1054 | | | | | | |
| AKT1S1 S183 vs | 6.42E−02 | 0.268251 | 391.5 | 323.5 | 28 | 17 |
| CDK13 S1054 | | | | | | |
| EIF4EBP2 T46 vs | 8.73E−02 | 0.279694 | 392.6 | 315.1 | 28 | 17 |
| DEPTOR S244 | | | | | | |
| PRKCD S302 vs | 2.97E−01 | 0.514535 | 382.6 | 354.1 | 24 | 21 |
| CDK13 S1054 | | | | | | |
| GSK3A T19 vs | 2.64E−01 | 0.468263 | 412.9 | 255.4 | 26 | 19 |
| DEPTOR S244 | | | | | | |
| PRKCD Y313 vs | 1.82E−01 | 0.423257 | 387.6 | 337.2 | 26 | 19 |
| CDK13 S1054 | | | | | | |
| STAT1 T719 vs | 1.39E−01 | 0.364932 | 388.2 | 339.1 | 27 | 18 |
| DEPTOR S244 | | | | | | |
| PRKCD S302 vs | 9.62E−01 | 0.96174 | 343.2 | 389.7 | 20 | 25 |
| MYC T58 | | | | | | |
| Ratio Sum PKC | 8.61E−01 | 0.926565 | 412.7 | 349.3 | 10 | 35 |
| GSK3 STAT1 | | | | | | |
| 4EBP2 AKT1S1 | | | | | | |
| vs CDK13 | | | | | | |
| ZNF608 0 | | | | | | |
| to 1 | | | | | | |
| EIF4EBP2 T46 vs | 4.18E−01 | 0.618157 | 441.4 | 335.9 | 14 | 31 |
| STAT5A S780 | | | | | | |
| EIF4EBP2 T46 vs | 2.49E−01 | 0.453812 | 467.3 | 334.2 | 11 | 34 |
| MYC T58 | | | | | | |
| AKT1S1 S183 vs | 3.55E−01 | 0.57207 | 331.6 | 432.9 | 25 | 20 |
| DEPTOR S244 | | | | | | |
| GSK3A T19 vs | 7.05E−01 | 0.847444 | 364.5 | 385.7 | 24 | 21 |
| CDK13 S1054 | | | | | | |
| AKT1S1 S183 vs | 4.70E−01 | 0.654898 | 363.3 | 362.4 | 19 | 26 |
| MYC T58 | | | | | | |
| GSK3A T19 vs | 7.20E−01 | 0.847444 | 272.9 | 390.8 | 14 | 31 |
| MYC T58 | | | | | | |
| PRKCD Y313 vs | 8.79E−01 | 0.931394 | 399.9 | 331.2 | 18 | 27 |
| MYC T58 | | | | | | |

### Example 3—Proteomic Signatures Associated with Responses to Midostaurin Plus Chemotherapy

To identify proteins that may be associated to, and thus predict, responses to midostaurin, we also performed a proteomic characterization of our AML sample set. As with the phosphoproteomics data, protein intensity values were normalized to a set of proteins with low variance across the experiment. These proteins are shown in Table 13. We first identified a set of proteins differentially expressed in extreme phenotypes, namely those that were increased or decreased in cells taken from patient with progression free survival of <6 with 24 months.

Proteins were selected by having a FDR<25% (probability true discovery>75%) by t-test in at least one comparison (i.e., in BM or PB samples). These proteins were used as the input of an ML model (model 1) trained using random forests (RF). Another model (model 2) was trained using proteins with unadjusted p-value<0.02 followed by feature selection using the boruta algorithm. A third model (model 3) used a combination of boruta selected proteins and correlated proteins as predictors. Protein predictors used to train these models are shown in Table 14.

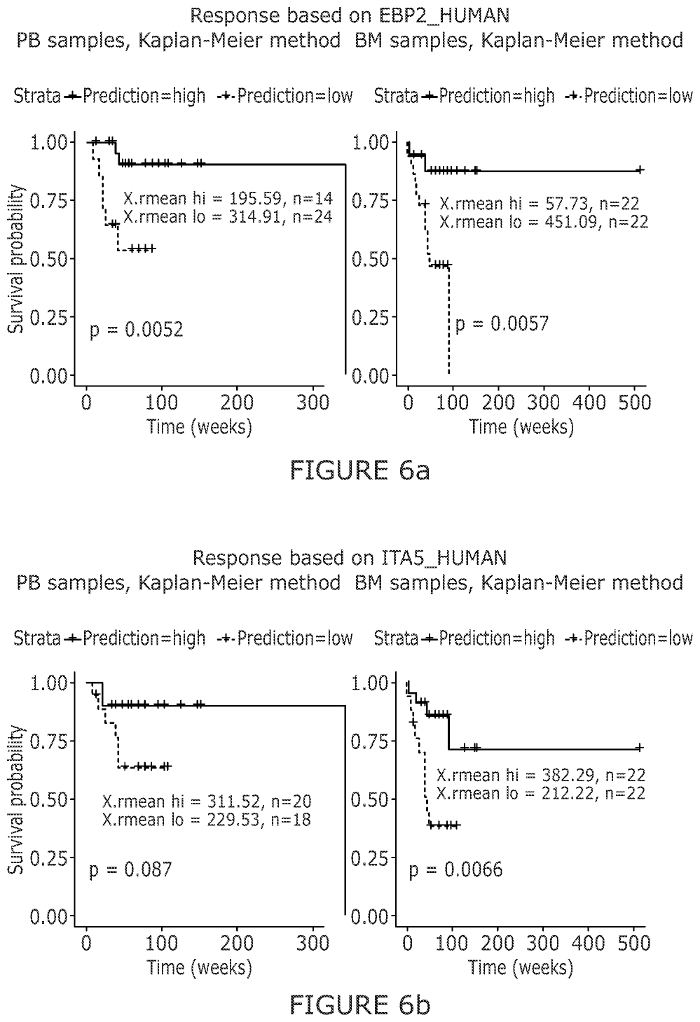

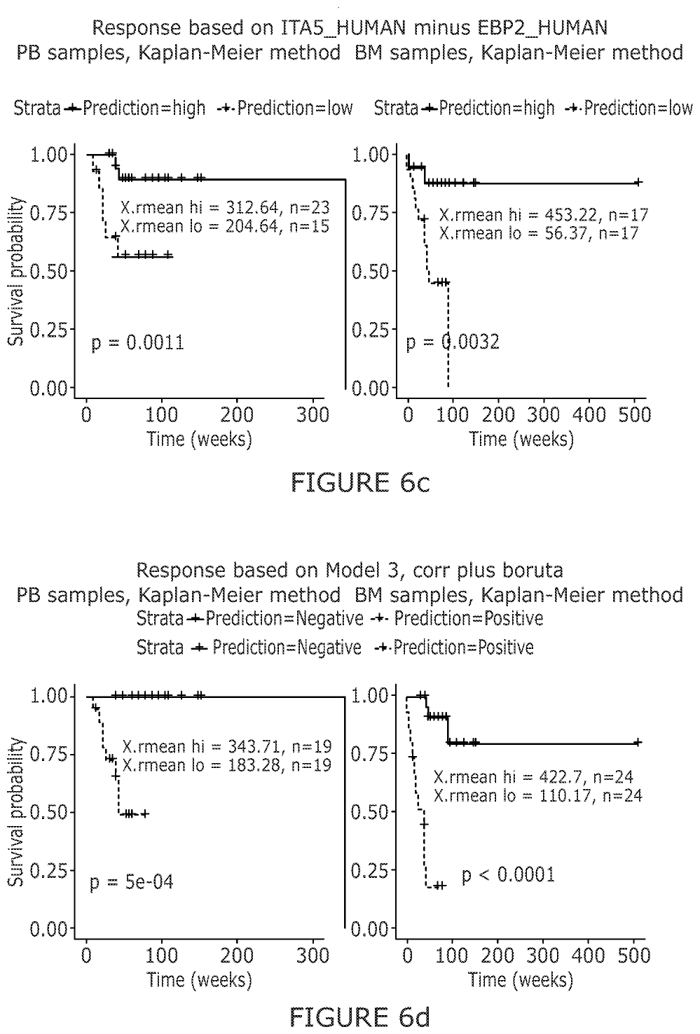

As with the analysis of phosphoproteomics data, proteins which, by themselves, could classify patients based on responses to midostaurin, were also used to derive ratios (i.e. response indices) to assess differential survival difference as a function of these ratios. Results of the differential survival analysis by single proteins, protein ratios and ML learning are shown in Tables 15 (for PB samples) and 16 (for BM samples). Single proteins that by themselves were predictive of responses included EBP2 and the integrin ITA5 (FIGS. 6a and 6b) and their ratio (FIG. 6c). All models were predictive of responses (as exemplified by model 3 in FIG. 6d).

Overall, these analyses uncovered a range of phosphorylation sites, proteins and computer predictive models that stratify patients based on their clinical responses to midostaurin plus chemotherapy. These molecular markers, by themselves or in combination, and algorithms could be used to predict patients more likely to respond to this treatment.

TABLE 13
Proteins used for normalization
Protein loading controls

B3GA2_HUMAN
NKD1_HUMAN
TLR9_HUMAN
PLCH1_HUMAN
PP6R1_HUMAN
PLAP_HUMAN
CLCA_HUMAN
UBXN1_HUMAN
TRI47_HUMAN
STX12_HUMAN

TABLE 14
Predictors in proteomic machine learning models

| models | predictors |
|---|---|
| Model1, corr | UAP1L_HUMAN; SPB1_HUMAN; JUNB_HUMAN; ITAS_HUMAN; IF2G_HUMAN; F16P1_HUMAN; |
| | GSHB_HUMAN; ADA_HUMAN; VATA_HUMAN; UN13D_HUMAN; REXO4_HUMAN; TRNT1_HUMAN; |
| | AT1A1_HUMAN; EBP2_HUMAN; VATB2_HUMAN; TLR2_HUMAN; BTBDG_HUMAN; TYY1_HUMAN; |
| | HMGB3_HUMAN; MGAT2_HUMAN; ARFP1_HUMAN; NGAP_HUMAN; CCL3_HUMAN |
| Model 2, boruta | EBP2_HUMAN; UN13D_HUMAN; VATB2_HUMAN; KALRN_HUMAN; VATA_HUMAN; MRCKB_HUMAN; |
| | GST2_HUMAN; RIOX1_HUMAN; S30BP_HUMAN; OSBL9_HUMAN; YIPF4_HUMAN; RECQ1_HUMAN; |
| | DNJC2_HUMAN; ECM1_HUMAN; STX12_HUMAN; SYRC_HUMAN; MEMO1_HUMAN; AL8A1_HUMAN; |
| | CAR19_HUMAN; IGL1_HUMAN; KCC1D_HUMAN; ABHD4_HUMAN; BFSP1_HUMAN; BMS1_HUMAN; |
| | RHG26_HUMAN; PLPL6_HUMAN; AGRA1_HUMAN; AMPD3_HUMAN |
| Model 3, corr plus | EBP2_HUMAN; UN13D_HUMAN; VATB2_HUMAN; KALRN_HUMAN; VATA_HUMAN; MRCKB_HUMAN; |
| boruta | GST2_HUMAN; RIOX1_HUMAN; S30BP_HUMAN; OSBL9_HUMAN; YIPF4_HUMAN; RECQ1_HUMAN; |
| | DNJC2_HUMAN; ECM1_HUMAN; STX12_HUMAN; SYRC_HUMAN; MEMO1_HUMAN; AL8A1_HUMAN; |
| | CAR19_HUMAN; IGL1_HUMAN; KCC1D_HUMAN; ABHD4_HUMAN; BFSP1_HUMAN; BMS1_HUMAN; |
| | RHG26_HUMAN; PLPL6_HUMAN; AGRA1_HUMAN; AMPD3_HUMAN; UAP1L_HUMAN; SPB1_HUMAN; |
| | JUNB_HUMAN; ITA5_HUMAN; IF2G_HUMAN; F16P1_HUMAN; GSHB_HUMAN; ADA_HUMAN; |
| | REXO4_HUMAN; TRNT1_HUMAN; AT1A1_HUMAN; TLR2_HUMAN; BTBDG_HUMAN; TYY1_HUMAN; |
| | HMGB3_HUMAN; MGAT2_HUMAN; ARFP1_HUMAN; NGAP_HUMAN; CCL3_HUMAN |

TABLE 15
Results of survival analysis using proteomics data based on PB samples

| signature | pvalue.PB | av.pb.hi | av.pb.low | n.pb.hi | n.pb.low |
|---|---|---|---|---|---|
| Protein marker ratios | | | | | |
| SYRC_HUMAN minus EBP2_HUMAN | 1.05E−03 | 327.91 | 187.52 | 23 | 15 |
| ITA5_HUMAN minus EBP2_HUMAN | 1.08E−02 | 312.64 | 204.64 | 23 | 15 |
| ITA5_HUMAN minus S30BP_HUMAN | 1.92E−03 | 329.08 | 183.99 | 22 | 16 |
| SYRC_HUMAN minus TRNT1_HUMAN | 1.42E−02 | 324.95 | 219.3 | 19 | 19 |
| VATB2_HUMAN minus TRNT1_HUMAN | 3.03E−02 | 320.59 | 227.07 | 18 | 20 |
| ITA5_HUMAN minus TRNT1_HUMAN | 2.94E−02 | 324.68 | 225.56 | 17 | 21 |
| SYRC_HUMAN minus S30BP_HUMAN | 2.86E−05 | 343.71 | 151.73 | 23 | 15 |
| VATB2_HUMAN minus EBP2_HUMAN | 5.40E−02 | 309.3 | 225.44 | 21 | 17 |
| VATB2_HUMAN minus S30BP_HUMAN | 5.40E−02 | 309.3 | 225.44 | 21 | 17 |
| UN13D_HUMAN minus RECQ1_HUMAN | 1.03E−01 | 305.91 | 238.8 | 18 | 20 |
| UN13D_HUMAN minus EBP2_HUMAN | 1.30E−01 | 297.6 | 232.8 | 23 | 15 |
| UN13D_HUMAN minus S30BP_HUMAN | 1.36E−01 | 297.6 | 233.8 | 22 | 16 |
| ITA5_HUMAN minus RECQ1_HUMAN | 1.22E−01 | 318.33 | 244.79 | 13 | 25 |
| SYRC_HUMAN minus RECQ1_HUMAN | 8.02E−02 | 321.96 | 239.57 | 15 | 23 |
| UN13D_HUMAN minus TRNT1_HUMAN | 2.43E−01 | 293.58 | 244.48 | 21 | 17 |
| VATB2_HUMAN minus RECQ1_HUMAN | 3.38E−02 | 322.24 | 229.38 | 17 | 21 |
| ML models | | | | | |
| Model 3, corr plus boruta | 4.98E−04 | 343.71 | 183.28 | 19 | 19 |
| Model1, corr | 4.96E−04 | 329.87 | 173.45 | 23 | 15 |
| Model 2, boruta | 2.60E−03 | 316.4 | 196.12 | 23 | 15 |
| Single protein markers | | | | | |
| EBP2_HUMAN | 5.22E−03 | 195.59 | 314.91 | 14 | 24 |
| REXO4_HUMAN | 1.73E−02 | 212.96 | 325.83 | 19 | 19 |
| BFSP1_HUMAN | 2.28E−03 | 343.71 | 204.43 | 18 | 20 |
| AL8A1_HUMAN | 2.41E−03 | 195.53 | 327.89 | 17 | 21 |
| S30BP_HUMAN | 3.86E−03 | 189.66 | 327.78 | 17 | 21 |
| SYRC_HUMAN | 1.05E−03 | 327.91 | 187.52 | 23 | 15 |
| GST2_HUMAN | 6.87E−02 | 312.23 | 222.3 | 21 | 17 |
| ITA5_HUMAN | 8.69E−02 | 311.52 | 229.53 | 20 | 18 |
| ARFP1_HUMAN | 1.06E−01 | 307.12 | 234.32 | 19 | 19 |
| VATB2_HUMAN | 8.40E−02 | 307.23 | 232.38 | 20 | 18 |
| CCL3_HUMAN | 3.06E−03 | 343.71 | 208.56 | 18 | 20 |
| RECQ1_HUMAN | 1.67E−01 | 249.14 | 316.03 | 26 | 12 |
| KALRN_HUMAN | 4.58E−02 | 231.73 | 323.41 | 22 | 16 |
| VATA_HUMAN | 2.20E−01 | 296.02 | 239.46 | 22 | 16 |
| YIPF4_HUMAN | 2.81E−01 | 287.04 | 252.02 | 23 | 15 |
| RIOX1_HUMAN | 2.37E−01 | 244.95 | 294.87 | 18 | 20 |
| ABHD4_HUMAN | 3.11E−01 | 287.53 | 245.17 | 23 | 15 |
| IGL1_HUMAN | 2.68E−01 | 301.64 | 247.47 | 15 | 23 |
| AT1A1_HUMAN | 1.19E−01 | 313.66 | 245.76 | 14 | 24 |
| RHG26_HUMAN | 2.78E−01 | 295.68 | 241.65 | 21 | 17 |
| TRNT1_HUMAN | 4.51E−01 | 257.44 | 289.42 | 20 | 18 |
| MRCKB_HUMAN | 3.73E−01 | 300.93 | 251.97 | 16 | 22 |
| ECM1_HUMAN | 5.38E−01 | 255.95 | 283.61 | 16 | 22 |
| MGAT2_HUMAN | 5.80E−01 | 263.43 | 283.34 | 22 | 16 |
| IF2G_HUMAN | 5.20E−01 | 286.92 | 257.39 | 18 | 20 |
| UN13D_HUMAN | 5.94E−01 | 280.65 | 258.39 | 22 | 16 |
| KCC1D_HUMAN | 1.79E−01 | 245.67 | 303.74 | 22 | 16 |
| MEMO1_HUMAN | 1.45E−01 | 232.06 | 306.98 | 20 | 18 |
| BTBDG_HUMAN | 6.60E−01 | 287.02 | 261.47 | 19 | 19 |
| DNJC2_HUMAN | 7.89E−01 | 278.77 | 265.3 | 16 | 22 |
| GSHB_HUMAN | 7.97E−01 | 256.29 | 279.58 | 17 | 21 |
| UAP1L_HUMAN | 6.57E−01 | 282.28 | 263.67 | 16 | 22 |
| PLPL6_HUMAN | 5,41E−01 | 261.49 | 285.79 | 21 | 17 |
| AMPD3_HUMAN | 8.92E−01 | 279.65 | 273.29 | 16 | 22 |
| OSBL9_HUMAN | 1.60E−01 | 243.95 | 305.08 | 22 | 16 |
| CAR19_HUMAN | 3.82E−01 | 249.74 | 291.01 | 19 | 19 |
| BMS1_HUMAN | 5.79E−01 | 286.87 | 258.7 | 19 | 19 |
| AGRA1_HUMAN | 9.45E−01 | 269.72 | 273.42 | 24 | 14 |
| STX12_HUMAN | 8.62E−01 | 273.74 | 270.87 | 19 | 19 |

TABLE 16
Results of survival analysis using proteomics data based on BM samples

| signature | pvalue.BM | av.bm.hi | av.bm.low | n.bm.hi | n.bm.low |
|---|---|---|---|---|---|
| Protein marker ratios | | | | | |
| SYRC_HUMAN minus EBP2_HUMAN | 1.48E−03 | 415.38 | 206.21 | 20 | 19 |
| ITA5_HUMAN minus EBP2_HUMAN | 3.20E−03 | 453.22 | 56.37 | 17 | 22 |
| ITA5_HUMAN minus S30BP_HUMAN | 1.26E−02 | 366.58 | 210.28 | 23 | 16 |
| SYRC_HUMAN minus TRNT1_HUMAN | 1.45E−03 | 435.56 | 225.86 | 18 | 21 |
| VATB2_HUMAN minus TRNT1_HUMAN | 1.18E−02 | 373.92 | 241.05 | 22 | 17 |
| ITA5_HUMAN minus TRNT1_HUMAN | 2.65E−02 | 420.56 | 169.69 | 17 | 22 |
| SYRC_HUMAN minus S30BP_HUMAN | 6.06E−02 | 369.88 | 263.68 | 18 | 21 |
| VATB2_HUMAN minus EBP2_HUMAN | 3.45E−02 | 361.19 | 247.85 | 23 | 16 |
| VATB2_HUMAN minus S30BP_HUMAN | 6.24E−02 | 352.12 | 255.84 | 23 | 16 |
| UN13D_HUMAN minus RECQ1_HUMAN | 1.84E−02 | 413.76 | 153.95 | 21 | 18 |
| UN13D_HUMAN minus EBP2_HUMAN | 1.28E−03 | 442.27 | 125.85 | 22 | 17 |
| UN13D_HUMAN minus S30BP_HUMAN | 3.55E−03 | 393.5 | 195.45 | 23 | 16 |
| ITA5_HUMAN minus RECQ1_HUMAN | 3.91E−02 | 379.13 | 255.8 | 18 | 21 |
| SYRC_HUMAN minus RECQ1_HUMAN | 1.26E−01 | 356.46 | 283.18 | 18 | 21 |
| UN13D_HUMAN minus TRNT1_HUMAN | 1.43E−02 | 419.35 | 144.24 | 22 | 17 |
| VATB2_HUMAN minus RECQ1_HUMAN | 2.71E−01 | 337.64 | 300.65 | 19 | 20 |
| ML models | | | | | |
| Model 3, corr plus boruta | 1.59E−07 | 422.7 | 110.17 | 24 | 15 |
| Model1, corr | 5.19E−06 | 460.41 | 156.49 | 19 | 20 |
| Model 2, boruta | 5.94E−04 | 384.59 | 208.19 | 23 | 16 |
| Single protein markers | | | | | |
| EBP2_HUMAN | 5.68E−03 | 57.73 | 451.09 | 22 | 17 |
| REXO4_HUMAN | 3.52E−03 | 228.62 | 411.49 | 20 | 19 |
| BFSP1_HUMAN | 2.16E−02 | 344.55 | 206.31 | 27 | 12 |
| AL8A1_HUMAN | 3.53E−02 | 195.56 | 395.88 | 17 | 22 |
| S30BP_HUMAN | 3.93E−02 | 253.3 | 339.93 | 17 | 22 |
| SYRC_HUMAN | 6.06E−02 | 369.88 | 263.68 | 18 | 21 |
| GST2_HUMAN | 1.41E−04 | 485.59 | 52.17 | 18 | 21 |
| ITA5_HUMAN | 6.61E−03 | 382.29 | 212.22 | 22 | 17 |
| ARFP1_HUMAN | 1.60E−02 | 425.04 | 164.51 | 17 | 22 |
| VATB2_HUMAN | 6.24E−02 | 352.12 | 255.84 | 23 | 16 |
| CCL3_HUMAN | 1.49E−01 | 350.14 | 266.82 | 21 | 18 |
| RECQ1_HUMAN | 3.40E−02 | 253.74 | 380.3 | 20 | 19 |
| KALRN_HUMAN | 1.96E−01 | 283.97 | 353.41 | 21 | 18 |
| VATA_HUMAN | 6.60E−02 | 347.19 | 267.67 | 24 | 15 |
| YIPF4_HUMAN | 1.19E−02 | 445.81 | 164.68 | 15 | 24 |
| RIOX1_HUMAN | 5.59E−02 | 218.39 | 406.81 | 20 | 19 |
| ABHD4_HUMAN | 9.03E−02 | 396.13 | 216.34 | 17 | 22 |
| IGL1_HUMAN | 1.51E−01 | 341.37 | 267.62 | 21 | 18 |
| AT1A1_HUMAN | 3.00E−01 | 344.77 | 279.98 | 20 | 19 |
| RHG26_HUMAN | 1.62E−01 | 392.62 | 182.5 | 17 | 22 |
| TRNT1_HUMAN | 1.96E−02 | 166.29 | 424.77 | 22 | 17 |
| MRCKB_HUMAN | 1.14E−01 | 416.44 | 180.96 | 16 | 23 |
| ECM1_HUMAN | 1.15E−03 | 200.85 | 382.92 | 17 | 22 |
| MGAT2_HUMAN | 1.74E−04 | 462.9 | 121.38 | 20 | 19 |
| IF2G_HUMAN | 6.88E−02 | 390.58 | 206.17 | 21 | 18 |
| UN13D_HUMAN | 2.08E−03 | 442.27 | 134.18 | 22 | 17 |
| KCC1D_HUMAN | 4.28E−01 | 308.53 | 314.69 | 20 | 19 |
| MEMO1_HUMAN | 5.13E−01 | 311.48 | 316.05 | 26 | 13 |
| BTBDG_HUMAN | 1.17E−02 | 157.48 | 413.54 | 19 | 20 |
| DNJC2_HUMAN | 3.72E−02 | 342.38 | 224.61 | 24 | 15 |
| GSHB_HUMAN | 5.47E−02 | 371.99 | 56.98 | 25 | 14 |
| UAP1L_HUMAN | 2.90E−01 | 354.72 | 202.28 | 21 | 18 |
| PLPL6_HUMAN | 4.23E−01 | 320.17 | 318.59 | 21 | 18 |
| AMPD3_HUMAN | 1.97E−01 | 251.2 | 374.22 | 21 | 18 |
| OSBL9_HUMAN | 9.29E−01 | 302.55 | 329.11 | 26 | 13 |
| CAR19_HUMAN | 7.53E−01 | 290.79 | 332.49 | 24 | 15 |
| BMS1_HUMAN | 6.17E−01 | 359.6 | 258.16 | 24 | 15 |
| AGRA1_HUMAN | 5.92E−01 | 280.47 | 354.44 | 23 | 16 |
| STX12_HUMAN | 8.61E−01 | 312.36 | 318.67 | 20 | 19 |

### Prophetic Example 4-Analysis of Specimens from FLT3 Mutant-Neoative AML Cases

Analysis of specimens from FLT3 mutant negative AML cases will be analysed in the same way as the FLT3 mutant-positive cases outlined above. Briefly, cells obtained from the peripheral blood and/or bone marrow samples from AML patients (previously determined to be FLT3 mutant-negative) will be lysed and protein extracted. An optional step includes culturing these cells for 2 to 3 hours in cell culture media containing foetal calf serum prior to lysis. Proteins will then be digested to obtain peptides, which will be purified from salts and other buffer components by reversed phase solid phase extraction. An aliquot of these peptides will be processed for phosphopeptide enrichment using chromatographic methods used for this purpose (such as those based on TiO₂, IMAC or ZrO₂).

Unmodified peptides and phosphopeptides obtained by sample digestion, desaltng and, optionally, phospho-enrichment will be analysed by LC-MS/MS. Extracted ion chromatograms of the peptides and phosphopeptides of interest will be constructed using in house or publically available software.

For this purpose, the mass-to-charge ratio (m/z) of the and the retention time un peptide or phosphopeptide markers will be used. Alternatively, the peptide and phosphopeptide markers can be quantified in the samples by selected reaction monitoring (SRM) or parallel reaction monitoring (PRM) where the tandem mass spectrometer is set up to quantify the fragments produced as a results of collision induced dissociation of the selected peptide m/z. The intensities of the peptide and phosphopeptide markers will then be correlated with the survival of the FLT3 mutant-negative patients from which the samples were obtained.

### Example 5—Defining Midostaurin Responsiveness in Cancer Patients Irrespective of their FLT-3 Mutant Status

### 1 Background and Aims

This Example provides a rationale behind separating patients in four different response groups based on the status of specific phosphorylation signatures following treatment with midostaurin plus chemotherapy.

### 2 Patient Grouping

### 2.1 Defining Positive and Negative Response to Midostaurin Treatment

A FLT3-mutant positive AML clinical dataset was assembled (samples access via the Princess Margaret Cancer Centre [PMCC], Toronto) to identify biomarkers of response to midostaurin+ chemotherapy. In this dataset, some patients were refractory (did not achieve complete remission[CR]) to this treatment, while several others achieved CR only to experience relapse after a certain point from the diagnosis day. For the purpose of this analysis, patients that responded positively (i.e. achieved CR) to the treatment (positive responders) were defined as those who did not experience relapse for 106 weeks or post diagnosis—treatment would have initiated upon confirmation of diagnosis. Patients that did not respond to treatment (negative responders) were defined as those who were refractory or experienced relapse within 26 weeks of diagnosis.

### 2.2 Identifying Multiple Biochemical Responses Amongst Positive Responders

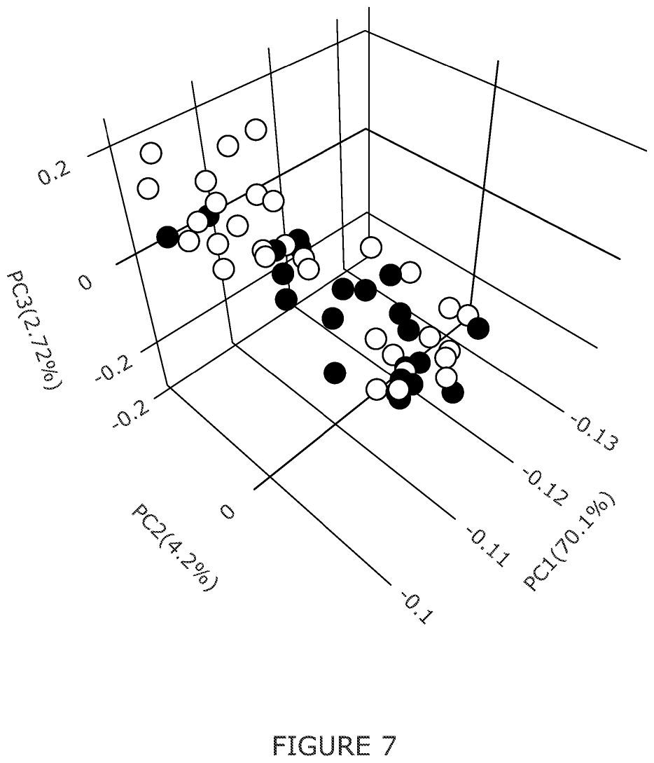

Analysis of phosphoproteomic data includes several types of so called multivariate analysis. In multivariate analyses, relationships between measurements are examined and visualised to elucidate trends in data and uncover relationships within them. One such analysis is principal component analysis (PCA) a technique for dimension reduction of large datasets and creates a ‘summary’ of core properties that can be both programatically and visually examined. PCA was performed on the PMCC clinical dataset and resulted in the plot shown in FIG. 7; each dot represents a patient (positive—grey; negative—dark grey), here patients with similar phosphopeptide measurements will cluster in the same plotting space, while distinct samples will occupy a distant location.

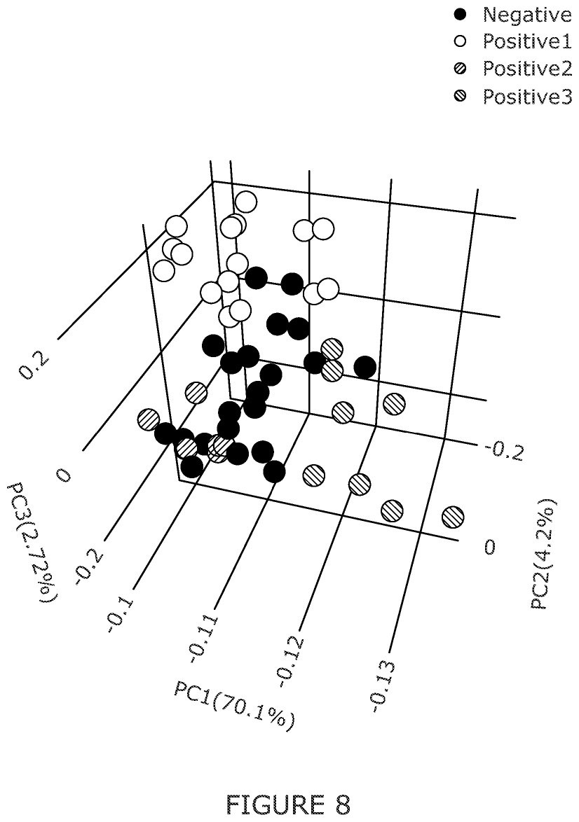

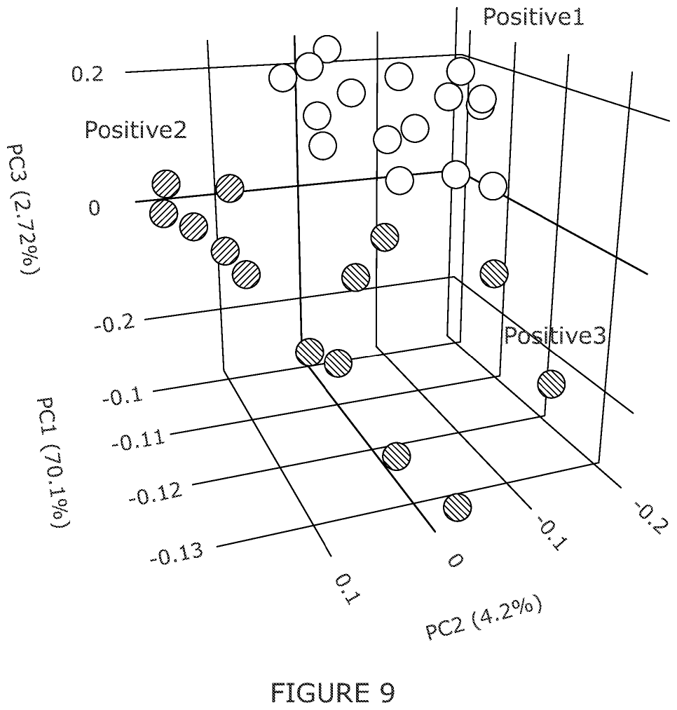

Assessment of the positive patient samples revealed a spectrum of phosphoproteomic responses, corresponding to differences in biochemical pathways, relative to negative patients. When the plot is oriented such that PC1 axis is parallel to the bottom of the plot, and PC3 axis is vertical, some of the positive patient samples are located below the bulk of negative patient samples, while others are located above. Patient samples were then grouped based their PCA behaviour (orientation of each data point on the PCA plot), and three distinct groups of positive patients were identified (FIGS. 8 and 9)—referred to as Positive 1, Positive 2 and Positive 3. For convenience and ease of interpretation, in FIG. 9 the same PCA plot was plotted without negative responders shown. This was computed including the negative samples.

### 2.3 Machine Learning

The patients that comprise the four response groups include patients that fall into the extreme phenotypes of AML patient response to midostaurin+ chemotherapy: no/poor response (negative; <26 weeks) and good response (positive 1, positive 2, positive 3; >106 weeks). A feature selection algorithm was then used to identify features (phosphorylation sites) that are important for defining and distinguishing between the patient response groups. The features resulting in the Panels A to D described above. Following feature selection, predictive models were built using a random forest approach to allow for classification of patient response to midostaurin treatment.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1.-46. (canceled)

47. A dosage regimen for cancer therapy, comprising administering to an individual subject midostaurin orally as either a 50 mg dose twice daily or a 100 mg dose twice daily,
wherein the individual subject is identified as suitable for treatment with midostaurin by determining a proteomic and/or a phosphoproteomic signature within a sample obtained from the individual subject wherein the proteomic and/or phosphoproteomic signature provides a personalised indication that the individual subject is a suitable candidate for treatment.

48. A method of treating cancer in a FLT3 mutant-negative or mutant-positive individual subject in need thereof, the method comprising
obtaining a sample from the individual subject and determining a phosphoproteomic signature within the sample obtained from the individual subject wherein the phosphoproteomic signature provides a personalised indication of whether dt the individual subject is a suitable candidate for treatment with midostaurin,
wherein if the individual subject is identified as suitable for treatment with midostaurin then treating the individual subject with midostaurin,
wherein if the individual subject is identified as unsuitable for treatment with midostaurin, then ceasing treatment with midostaurin.

49. The method as claimed in claim 48, wherein the phosphoproteomic signature comprises quantifying the amount of a post-translational modification via phosphorylation at a site within one or more proteins present in the sample.

50. The method as claimed in claim 49, wherein the phosphoproteomic signature further comprises a determination of an amount of phosphorylation at a first phosphorylation site within the one or more proteins and comparing it with an amount of phosphorylation at a second phosphorylation site within the one or more proteins.

51. The method as claimed in claim 50, wherein the determination comprises:
(i) dividing the amount phosphorylation at the first phosphorylation site by the amount of phosphorylation at the second phosphorylation site; or
(ii) subtracting from the amount of phosphorylation at the first phosphorylation site from the amount of phosphorylation at the second phosphorylation site.

52. The method as claimed in claim 49, wherein step of quantifying the amount of a post-translational modification via phosphorylation at a site within one or more proteins present in the sample comprises performing an in vitro assay to detect the amount of phosphorylation at the site in the sample.

53. The method as claimed in claim 52 wherein the in vitro assay is selected from one or more of the group consisting of:
an LC-MS/MS assay; and an assay based on an affinity reagent.

54. The method as claimed in claim 53, wherein the assay based on an affinity reagents comprises one or more affinity reagents selected from:
an aptamer; a molecularly imprinted polymers; and an antibody or an antigen binding fragment or mimetic thereof.

55. The method as claimed in claim 54, wherein the assay is selected from:
a Western blot assay; an ELISA assay; a reversed phase protein assay; and a lateral flow antigen testing assay.

56. The method as claimed in claim 49, wherein the phosphorylation at a site within one or more proteins comprises the post-translational modifications defined in any one of Panels A to C, as set out below:
Panel A:

| Phosphor- | | | |
|---|---|---|---|
| ylation | Uniprot | | Ion |
| site | ID | Protein name | Sequence |
| TP53BP1 | TP53B_ | TP53-binding | STPFIVPS |
| (T382); | HUMAN | protein 1 | SPTEQEGR |
| | | (53BP1) | [SEQ ID |
| | | (p53-binding | NO: 1] |
| | | protein 1) | |
| | | (p53BP1) | |
| MLF2 | MLF2_ | Myeloid leukemia | LAIQGPED |
| (S240); | HUMAN | factor 2 | SPSR |
| | | (Myelodysplasia- | [SEQ ID |
| | | myeloid leukemia | NO: 2] |
| | | factor 2) | |
| MEFV | MEFV_ | Pyrin | SLEVTIST |
| (S248); | HUMAN | (Marenostrin) | GEK |
| | | | [SEQ ID |
| | | | NO: 3] |
| AHNAK | AHNK_ | Neuroblast | VSMPDVEL |
| (S3426); | HUMAN | differentiation- | NLKSPK |
| | | associated | [SEQ ID |
| | | protein AHNAK | NO: 4] |
| | | (Desmoyokin) | |
| NHSL2 | NHSL2_ | NHS-like | SVSLVKDE |
| (S210); | HUMAN | protein 2 | PGLLPEGG |
| | | | SALPK |
| | | | [SEQ ID |
| | | | NO: 5] |

or
Panel B:

| Phosphor- | | | |
|---|---|---|---|
| ylation | Uniprot | Protein | Ion |
| site | ID | name | Sequence |
| SYK | KSYK_ | Tyrosine-protein | IKSYSFPK |
| (S295); | HUMAN | kinase SYK (EC | PGHR |
| SYK | | 2.7.10.2) (Spleen | [SEQ ID |
| (S297); | | tyrosine kinase) | NO: 6] |
| | | (p72-Syk) | |
| LARP1 | LARP1_ | La-related protein | ESPRPLQL |
| (S90); | HUMAN | 1 (La ribonucleo- | PGAEGPAI |
| | | protein domain | SDGEEGGG |
| | | family member 1) | EPGAGGGA |
| | | | AGAAGAGR |
| | | | [SEQ ID |
| | | | NO: 7] |
| SVIL | SVIL_ | Supervillin | QAHDLSPA |
| (S228); | HUMAN | (Archvillin) | AESSSTFS |
| | | (p205/p250) | FSGR |
| | | | [SEQ ID |
| | | | NO: 8] |
| CCDC86 | CCD86_ | Coiled-coil domain- | LGGLRPES |
| (S24); | HUMAN | containing protein | PESLTSVS |
| | | 86 (Cytokine-in- | R |
| | | duced protein with | [SEQ ID |
| | | coiled-coil domain) | NO: 9] |
| PFKFB2 | F262_ | 6-phosphofructo-2- | NYSVGSRP |
| (S486); | HUMAN | kinase/fructose- | LKPLSPLR |
| | | 2,6-bisphosphatase | [SEQ ID |
| | | 2 (6PF-2-K/Fru- | NO: 10] |
| | | 2,6-P2ase 2) | |
| | | (PFK/FBPase 2) | |
| | | (6PF-2-K/Fru-2,6- | |
| | | P2ase heart-type | |
| | | isozyme) [Includes: | |
| | | 6-phosphofructo-2- | |
| | | kinase (EC | |
| | | 2.7.1.105); | |
| | | Fructose-2,6- | |
| | | bisphosphatase | |
| | | (EC 3.1.3.46)] | |

or
Panel C

| Phosphor- | | | |
|---|---|---|---|
| ylation | Uniprot | Protein | Ion |
| site | ID | name | Sequence |
| SHANK3 | SHAN3_ | SH3 and multiple | SRSPSPSP |
| (S1650); | HUMAN | ankyrin repeat | LPSPASGP |
| SHANK3 | | domains protein 3 | GPGAPGPR |
| (S1654); | | (Shank3) (Proline- | [SEQ ID |
| | | rich synapse- | NO: 11] |
| | | associated protein | |
| | | 2) (ProSAP2) | |
| SRRM2 | SRRM2_ | Serine/arginine | SRTPLISR |
| (S1878); | HUMAN | repetitive | [SEQ ID |
| SRRM2 | | matrix protein 2 | NO: 12] |
| (T1880); | | (300 kDa nuclear | |
| | | matrix antigen) | |
| | | (Serine/arginine- | |
| | | rich splicing | |
| | | factor-related | |
| | | nuclear matrix | |
| | | protein of 300 | |
| | | kDa) (SR-related | |
| | | nuclear matrix | |
| | | protein of 300 | |
| | | kDa) (Ser/Arg- | |
| | | related nuclear | |
| | | matrix protein | |
| | | of 300 kDa) | |
| | | (Splicing coac- | |
| | | tivator subunit | |
| | | SRm300) (Tax- | |
| | | responsive | |
| | | enhancer | |
| | | element-binding | |
| | | protein 803) | |
| | | (TaxREB803) | |
| KIAA0528 | C2CD5_ | C2 domain- | NQTYSFSP |
| (S297); | HUMAN | containing | SK |
| | | protein 5 (C2 | [SEQ ID |
| | | domain- | NO: 13] |
| | | containing | |
| | | phosphoprotein | |
| | | of 138 kDa) | |
| KIAA1429 | VIR_ | Protein virilizer | VISHDRDS |
| (S138); | HUMAN | homolog | PPPPPPPP |
| | | | PPPQPQPS |
| | | | LK |
| | | | [SEQ ID |
| | | | NO: 14] |
| MAP1A | MAP1A_ | Microtubule- | SLSPEDAE |
| (S1157); | HUMAN | associated protein | SLSVLSVP |
| | | 1A (MAP-1A) | SPDTANQE |
| | | (Proliferation- | PTPK |
| | | related protein | [SEQ ID |
| | | p80) [Cleaved | NO: 15] |
| | | into: MAP1A heavy | |
| | | chain; MAP1 light | |
| | | chain LC2] | |

and
wherein phosphorylation at the sites defined within any one Panels A to C is predictive that midostaurin is effective in treating the individual subject.

57. The method as claimed in claim 48, wherein the method further comprises determining a proteomic signature within the sample obtained from the individual subject wherein the proteomic signature provides a personalised indication that the individual subject is a suitable candidate for treatment, and wherein determining the proteomic signature comprises quantifying the amount of one or more proteins present in the sample whose abundance is correlated to efficacy of midostaurin treatment.

58. The method as claimed in claim 57, wherein the proteomic signature further comprises quantifying the amount of one or more proteins present in the sample, wherein the one or more proteins are as defined below:
Heterogeneous nuclear ribonucleoprotein M
Protein PML (E3 SUMO-protein ligase PML) (EC 2.3.2.-) (Promyelocytic leukemia protein) (RING finger protein 71) (RING-type E3 SUMO transferase PML) (Tripartite motif-containing protein 19) (TRIM19)
Neuroblast differentiation-associated protein AHNAK
Myoferlin
Dedicator of cytokinesis protein 10 (Zizimin-3)
Eukaryotic translation initiation factor 3 subunit D
Glutamine-fructose-6-phosphate aminotransferase
Choline-phosphate cytidylyltransferase A
V-type proton ATPase subunit B, brain isoform
Eukaryotic translation initiation factor 2 subunit 3
V-type proton ATPase catalytic subunit A

59. The method as claimed in claim 48, wherein the midostaurin is administered as part of a combination therapy.

60. The method as claimed in claim 59, wherein the combination therapy comprises chemotherapy, optionally wherein the chemotherapy comprises one or more of the group consisting of: cytarabine, doxorubicin, idarubicin and/or daunorubicin.

61. The method as claimed in claim 48, further comprising administering to an individual subject midostaurin orally as either a 50 mg dose twice daily or a 100 mg dose twice daily,

62.-69. (canceled)

70. The method of claim 48, wherein the cancer is selected from the group consisting of: acute myeloid leukemia (AML); high-risk myeloid dysplastic syndrome (MDS); aggressive systemic mastocytosis (ASM); systemic mastocytosis with associated hematological neoplasm (SM-AHN); and mast cell leukemia (MCL).

71. A method of treating cancer in a FLT3 mutant-negative or mutant-positive individual subject in need thereof, the method comprising administering midostaurin to a subject determined to have increased amount of a phosphorylation at a site selected from Panels A to C, as set out below:
Panel A:

| Phosphor- | | | |
|---|---|---|---|
| ylation | Uniprot | Protein | Ion |
| site | ID | name | Sequence |
| TP53BP1 | TP53B_ | TP53-binding | STPFIVPS |
| (T382); | HUMAN | protein 1 | SPTEQEGR |
| | | (53BP1) (p53- | [SEQ ID |
| | | binding protein | NO: 1] |
| | | 1) (p53BP1) | |
| MLF2 | MLF2_ | Myeloid leukemia | LAIQGPED |
| (S240); | HUMAN | factor 2 | SPSR |
| | | (Myelodysplasia- | [SEQ ID |
| | | myeloid leukemia | NO: 2] |
| | | factor 2) | |
| MEFV | MEFV_ | Pyrin | SLEVTIST |
| (S248); | HUMAN | (Marenostrin) | GEK |
| | | | [SEQ ID |
| | | | NO: 3] |
| AHNAK | AHNK_ | Neuroblast dif- | VSMPDVEL |
| (S3426); | HUMAN | ferentiation- | NLKSPK |
| | | associated | [SEQ ID |
| | | protein AHNAK | NO: 4] |
| | | (Desmoyokin) | |
| NHSL2 | NHSL2_ | NHS-like | SVSLVKDE |
| (S210); | HUMAN | protein 2 | PGLLPEGG |
| | | | SALPK |
| | | | [SEQ ID |
| | | | NO: 5] |

or
Panel B:

| Phosphor- | | | |
|---|---|---|---|
| ylation | Uniprot | Protein | Ion |
| site | ID | name | Sequence |
| SYK | KSYK_ | Tyrosine-protein | IKSYSFPK |
| (S295); | HUMAN | kinase SYK (EC | PGHR |
| SYK | | 2.7.10.2) | [SEQ ID |
| (S297); | | (Spleen tyrosine | NO: 6] |
| | | kinase) | |
| | | (p72-Syk) | |
| LARP1 | LARP1_ | La-related | ESPRPLQL |
| (S90); | HUMAN | protein 1 (La | PGAEGPAI |
| | | ribonucleopro- | SDGEEGGG |
| | | tein domain | EPGAGGGA |
| | | family member | AGAAGAGR |
| | | 1) | [SEQ ID |
| | | | NO: 7] |
| SVIL | SVIL_ | Supervillin | QAHDLSPA |
| (S228); | HUMAN | (Archvillin) | AESSSTFS |
| | | (p205/p250) | FSGR |
| | | | [SEQ ID |
| | | | NO: 8] |
| CCDC86 | CCD86_ | Coiled-coil | LGGLRPES |
| (S24); | HUMAN | domain- | PESLTSVS |
| | | containing | R |
| | | protein 86 | [SEQ ID |
| | | (Cytokine- | NO: 9] |
| | | induced | |
| | | protein with | |
| | | coiled-coil | |
| | | domain) | |
| PFKFB2 | F262_ | 6-phospho- | NYSVGSRP |
| (S486); | HUMAN | fructo-2- | LKPLSPLR |
| | | kinase/ | [SEQ ID |
| | | fructose-2,6- | NO: 10] |
| | | bisphosphatase | |
| | | 2(6PF-2-K/Fru- | |
| | | 2,6-P2ase 2) | |
| | | (PFK/FBPase | |
| | | 2) (6PF-2-K/ | |
| | | Fru-2,6-P2ase | |
| | | heart-type | |
| | | isozyme) | |
| | | [Includes: 6- | |
| | | phosphofructo- | |
| | | 2-kinase (EC | |
| | | 2.7.1.105); | |
| | | Fructose-2,6- | |
| | | bisphosphatase | |
| | | (EC 3.1.3.46)] | |

or
Panel C

| Phosphor- | | | |
|---|---|---|---|
| ylation | Uniprot | Protein | Ion |
| site | ID | name | Sequence |
| SHANK3 | SHAN3_ | SH3 and multiple | SRSPSPSP |
| (S1650); | HUMAN | ankyrin repeat | LPSPASGP |
| SHANK3 | | domains protein | GPGAPGPR |
| (S1654); | | 3 (Shank3) | [SEQ ID |
| | | (Proline-rich | NO: 11] |
| | | synapse-as- | |
| | | sociated | |
| | | protein 2) | |
| | | (ProSAP2) | |
| SRRM2 | SRRM2_ | Serine/arginine | SRTPLISR |
| (S1878); | HUMAN | repetitive | [SEQ ID |
| SRRM2 | | matrix protein | NO: 12] |
| (T1880); | | 2 (300 kDa | |
| | | nuclear matrix | |
| | | antigen) | |
| | | (Serine/arginine- | |
| | | rich splicing | |
| | | factor-related | |
| | | nuclear matrix | |
| | | protein of | |
| | | 300 kDa) (SR- | |
| | | related nuclear | |
| | | matrix protein of | |
| | | 300 kDa) (Ser/ | |
| | | Arg-related | |
| | | nuclear matrix | |
| | | protein of 300 | |
| | | kDa) (Splicing | |
| | | coactivator | |
| | | subunit SRm300) | |
| | | (Tax-responsive | |
| | | enhancer | |
| | | element-binding | |
| | | protein 803) | |
| | | (TaxREB803) | |
| KIAA0528 | C2CD5_ | C2 domain- | NQTYSFSP |
| (S297); | HUMAN | containing | SK |
| | | protein 5 (C2 | [SEQ ID |
| | | domain-containing | NO: 13] |
| | | phosphoprotein | |
| | | of 138 kDa) | |
| KIAA1429 | VIR_ | Protein virilizer | VISHDRDS |
| (S138); | HUMAN | homolog | PPPPPPPP |
| | | | PPPQPQPS |
| | | | LK |
| | | | [SEQ ID |
| | | | NO: 14] |
| MAP1A | MAP1A_ | Microtubule- | SLSPEDAE |
| (S1157); | HUMAN | associated | SLSVLSVP |
| | | protein 1A | SPDTANQE |
| | | (MAP-1A) | PTPK |
| | | (Proliferation- | [SEQ ID |
| | | related | NO: 15] |
| | | protein p80) | |
| | | [Cleaved into: | |
| | | MAP1A heavy | |
| | | chain; MAP1 | |
| | | light chain LC2] | |
